SEQUENCE LISTING (153 sequences)

SEQ ID NO: 1 - DNA, 22 bp, Artificial Sequence
  Description of Artificial Sequence Synthetic oligonucleotide
ccagccgaccgattccgtcatg

SEQ ID NO: 2 - DNA, 22 bp, Artificial Sequence
  Description of Artificial Sequence Synthetic oligonucleotide
tattcctaatcactatgtcgag

SEQ ID NO: 3 - DNA, 22 bp, Artificial Sequence
  Description of Artificial Sequence Synthetic oligonucleotide
tattcctcatggagtgattatg

SEQ ID NO: 4 - DNA, 22 bp, Artificial Sequence
  Description of Artificial Sequence Synthetic oligonucleotide
cgagatgtcatgaaagagatgg

SEQ ID NO: 5 - DNA, 22 bp, Artificial Sequence
  Description of Artificial Sequence Synthetic oligonucleotide
ccctctgtgtgctgaagggggg

SEQ ID NO: 6 - DNA, 22 bp, Artificial Sequence
  Description of Artificial Sequence Synthetic oligonucleotide
tttgctgacctgctggattaca

SEQ ID NO: 7 - DNA, 22 bp, Artificial Sequence
  Description of Artificial Sequence Synthetic oligonucleotide
tgacctgctggattacattaaa

SEQ ID NO: 8 - DNA, 22 bp, Artificial Sequence
  Description of Artificial Sequence Synthetic oligonucleotide
catgactgtagattttatcaga

SEQ ID NO: 9 - DNA, 22 bp, Artificial Sequence
  Description of Artificial Sequence Synthetic oligonucleotide
tgacactggtaaaacaatgcaa

SEQ ID NO: 10 - DNA, 22 bp, Artificial Sequence
  Description of Artificial Sequence Synthetic oligonucleotide
ctttccctggtcaagcggtaca

SEQ ID NO: 11 - DNA, 22 bp, Artificial Sequence
  Description of Artificial Sequence Synthetic oligonucleotide
tttccctggtcaagcggtacaa

SEQ ID NO: 12 - DNA, 22 bp, Artificial Sequence
  Description of Artificial Sequence Synthetic oligonucleotide
ttggatttgaaattccagacaa

SEQ ID NO: 13 - DNA, 22 bp, Artificial Sequence
  Description of Artificial Sequence Synthetic oligonucleotide
tgtcattagtgaaactgggaaa

SEQ ID NO: 14 - DNA, 22 bp, Artificial Sequence
  Description of Artificial Sequence Synthetic oligonucleotide
taagatgagagttcaagttgaa

SEQ ID NO: 15 - DNA, 1301 bp, Criteculus sp.
ttacctcaccgctttctcgtgcctcggcgcctcctctgcgggcttcctcctcacaccgctcttcgctcccgccagccgaccgattccgtcatggcgacccgcagccccagcgtcgtgattagtgatgatgaaccaggctatgacctagatttattttgtattcctaatcactatgtcgaggatttggaaaaggtgtttattcctcatggagtgattatggacaggactgaaagacttgcccgagatgtcatgaaagagatgggaggccatcacattgtggccctctgtgtgctgaaggggggctataaattctttgctgacctgctggattacattaaagcactgaatagaaatagtgatagatccattcccatgactgtagattttatcagactgaagagctactgtaatgatcagtcaacaggggacataaaagttattggtggggatgatctctcaactttaactggaaagaatgtcttgattgttgaggacataattgacactggtaaaacaatgcaaactctgctttccctggtcaagcggtacaacctcaaaatggttaaggttgcaagcttgctggtgaaaaggacctctcgaagtgttggatataggccagactttgttggatttgaaattccagacaagtttgttgttggatatgcccttgactataatgagtacttcagggatttgaatcatatttgtgtcattagtgaaactgggaaagccaaatacaaagcctaagatgagagttcaagttgaatctgcaaacacgaggagtcccattcatgttcccagtaaaattaccaagcattctagttctgcagccatctgcttagtacagctttttgcatgaaccttctaagaattttatggttttttatttttagaaatgtcagttgctgcattcttaaactttttatttgcactatgagccttcgatagattgtcgcttaccttgtgagcaaagtaaatctcttaaattaccactattaaaaatgctgagattgtatctgtaagaaacatttaaagaggagatatattagttttttaattggtattttaatttttatatattcgggaaagaaagctgtgattgaatattgttaattataccaccatgtgtttagaacagtgtgaagcactcagtttcatgtcagtaaccatctaagaagttttgctcagtggaataaacatatgttttatcagtgttgcctatattttcccacttggattctttcagtgagtcattgtcaacagttcctttaaatgcaaataaattctaaaaatttaccact

SEQ ID NO: 16 - DNA, 24 bp, Artificial Sequence
  Description of Artificial Sequence Synthetic oligonucleotide
tcaaaacgtcgtacgacgttttga

SEQ ID NO: 17 - DNA, 24 bp, Artificial Sequence
  Description of Artificial Sequence Synthetic oligonucleotide
tcgagacgtcgtacgacgtctcga

SEQ ID NO: 18 - DNA, 24 bp, Artificial Sequence
  Description of Artificial Sequence Synthetic oligonucleotide
tccatacgtcgtacgacgtatgga

SEQ ID NO: 19 - DNA, 24 bp, Artificial Sequence
  Description of Artificial Sequence Synthetic oligonucleotide
tcaaaaccttgtacaaggttttga

SEQ ID NO: 20 - DNA, 22 bp, Artificial Sequence
  Description of Artificial Sequence Synthetic oligonucleotide
cgagatgtcgtacaagagatgg

SEQ ID NO: 21 - DNA, 22 bp, Artificial Sequence
  Description of Artificial Sequence Synthetic oligonucleotide
cgagatgtcgtacgacatctcg

SEQ ID NO: 22 - DNA, 22 bp, Artificial Sequence
  Description of Artificial Sequence Synthetic oligonucleotide
ccatctcttgtacaagagatgg

SEQ ID NO: 23 - DNA, 50 bp, Artificial Sequence
  Description of Artificial Sequence Synthetic oligonucleotide
tggcatacaagtttcgagatgtcgtacgacatctcgacaatcgtctgtca

SEQ ID NO: 24 - protein, 163 aa, Artificial Sequence
  Description of Artificial Sequence Synthetic polypeptide
MNTKYNKEFLLYLAGFVDGDGSIIAQIKPNQDTKFKHQLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 25 - protein, 163 aa, Artificial Sequence
  Description of Artificial Sequence Synthetic polypeptide
MNTKYNKEFLLYLAGFVDGDGSIIAQIKPNQETKFKHQLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 26 - protein, 163 aa, Artificial Sequence
  Description of Artificial Sequence Synthetic polypeptide
MNTKYNKEFLLYLAGFVDGDGSIIAQIKPNQPAKFKHQLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 27 - protein, 163 aa, Artificial Sequence
  Description of Artificial Sequence Synthetic polypeptide
MNTKYNKEFLLYLAGFVDGDGSIIAQIKPNQRDKFKHQLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 28 - protein, 163 aa, Artificial Sequence
  Description of Artificial Sequence Synthetic polypeptide
MNTKYNKEFLLYLAGFVDGDGSIIAQIKPNQSCKFKHKLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 29 - protein, 163 aa, Artificial Sequence
  Description of Artificial Sequence Synthetic polypeptide
MNTKYNKEFLLYLAGFVDGDGSIIAQIKPNQSCKFKHSLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 30 - protein, 163 aa, Artificial Sequence
  Description of Artificial Sequence Synthetic polypeptide
MNTKYNKEFLLYLAGFVDGDGSIIAQIKPNQSCKFKHTLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 31 - protein, 163 aa, Artificial Sequence
  Description of Artificial Sequence Synthetic polypeptide
MNTKYNKEFLLYLAGFVDGDGSIIAQIKPNQSGKFKHALSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 32 - protein, 163 aa, Artificial Sequence
  Description of Artificial Sequence Synthetic polypeptide
MNTKYNKEFLLYLAGFVDGDGSIIAQIKPNQSHKFKHQLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 33 - protein, 163 aa, Artificial Sequence
  Description of Artificial Sequence Synthetic polypeptide
MNTKYNKEFLLYLAGFVDGDGSIIAQIKPNQSPKFKHHLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 34 - protein, 163 aa, Artificial Sequence
  Description of Artificial Sequence Synthetic polypeptide
MNTKYNKEFLLYLAGFVDGDGSIIAQIKPNQSPKFKHQLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 35 - protein, 163 aa, Artificial Sequence
  Description of Artificial Sequence Synthetic polypeptide
MNTKYNKEFLLYLAGFVDGDGSIIAQIKPNQSQKFKHQLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 36 - protein, 163 aa, Artificial Sequence
  Description of Artificial Sequence Synthetic polypeptide
MNTKYNKEFLLYLAGFVDGDGSIIAQIKPNQSQKFKHYLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 37 - protein, 163 aa, Artificial Sequence
  Description of Artificial Sequence Synthetic polypeptide
MNTKYNKEFLLYLAGFVDGDGSIIAQIKPNQSRKFKHQLYLTFQVTQKTQRRWFLDKLVDEIGVGYVRDSGSVSNYILSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 38 - protein, 163 aa, Artificial Sequence
  Description of Artificial Sequence Synthetic polypeptide
MNTKYNKEFLLYLAGFVDGDGSIIAQIKPNQSSKFKHDLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 39 - protein, 163 aa, Artificial Sequence
  Description of Artificial Sequence Synthetic polypeptide
MNTKYNKEFLLYLAGFVDGDGSIIAQIKPNQSTKFKHGLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 40 - protein, 163 aa, Artificial Sequence
  Description of Artificial Sequence Synthetic polypeptide
MNTKYNKEFLLYLAGFVDGDGSIIAQIKPNQSTKFKHNLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 41 - protein, 163 aa, Artificial Sequence
  Description of Artificial Sequence Synthetic polypeptide
MNTKYNKEFLLYLAGFVDGDGSIIAQIKPNQSTKFKHSLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 42 - protein, 163 aa, Artificial Sequence
  Description of Artificial Sequence Synthetic polypeptide
MNTKYNKEFLLYLAGFVDGDGSIIAQIKPNQSTKFKHTLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 43 - protein, 163 aa, Artificial Sequence
  Description of Artificial Sequence Synthetic polypeptide
MNTKYNKEFLLYLAGFVDGDGSIIAQIKPNQSVKFKHHLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 44 - protein, 163 aa, Artificial Sequence
  Description of Artificial Sequence Synthetic polypeptide
MNTKYNKEFLLYLAGFVDGDGSIIAQIKPNQTPKFKHQLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 45 - protein, 163 aa, Artificial Sequence
  Description of Artificial Sequence Synthetic polypeptide
MNTKYNKEFLLYLAGFVDGDGSIIAQIKPNQTTKFKHQLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 46 - protein, 163 aa, Artificial Sequence
  Description of Artificial Sequence Synthetic polypeptide
MNTKYNKEFLLYLAGFVDGDGSIIAQIKPTQSTKFKHNLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 47 - protein, 163 aa, Artificial Sequence
  Description of Artificial Sequence Synthetic polypeptide
MNTKYNKEFLLYLAGFVDGDGSIIAQITPNQSRKFKHQLRLTFQVTQKTQRRWFLDKLVDEIGVGYVRDSGSVSNYILSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 48 - DNA, 26 bp, Artificial Sequence
  Description of Artificial Sequence Synthetic oligonucleotide
gcaactttagtgctgacacatacagg

SEQ ID NO: 49 - DNA, 24 bp, Artificial Sequence
  Description of Artificial Sequence Synthetic oligonucleotide
acaaccttgattggagacttgacc

SEQ ID NO: 50 - DNA, 15 bp, Artificial Sequence
  Description of Artificial Sequence Synthetic oligonucleotide
ctannnttgaccttt

SEQ ID NO: 51 - DNA, 15 bp, Artificial Sequence
  Description of Artificial Sequence Synthetic oligonucleotide
aaaggtcaannntag

SEQ ID NO: 52 - protein, 163 aa, Artificial Sequence
  Description of Artificial Sequence Synthetic polypeptide
MNTKYNKEFLLYLAGFVDGDGSIIAQIKPNQTHKFKHQLSLTFRVTQKTQRRWFLDKLVDEIGVGYVADSGSVSNYNLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 53 - protein, 163 aa, Artificial Sequence
  Description of Artificial Sequence Synthetic polypeptide
MNTKYNKEFLLYLAGFVDGDGSIIAQIKPSQSQKFKHQLSLTFRVTQKTQRRWFLDKLVDEIGVGYVYDSGSVSDYNLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 54 - protein, 163 aa, Artificial Sequence
  Description of Artificial Sequence Synthetic polypeptide
MNTKYNKEFLLYLAGFVDGDGSIIAQIKPNQTHKFKHQLSLTFRVTQKTQRRWFLDKLVDEIGVGYVYDSGSVSDYNLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 55 - protein, 163 aa, Artificial Sequence
  Description of Artificial Sequence Synthetic polypeptide
MNTKYNKEFLLYLAGFVDGDGSIIAQIKPNQTHKFKHQLSLTFRVTQKTQRRWFLDKLVDEIGVGYVYDSGSVSDYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 56 - protein, 163 aa, Artificial Sequence
  Description of Artificial Sequence Synthetic polypeptide
MNTKYNKEFLLYLAGFVDGDGSIIAQIKPNQSTKFKHYLSLTFKVTQKTQRRWFLDKLVDEIGVGYVYDSGSVSEYVLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 57 - protein, 163 aa, Artificial Sequence
  Description of Artificial Sequence Synthetic polypeptide
MNTKYNKEFLLYLAGFVDGDGSIIAQIKPNQRDKFKHQLSLTFKVTQKTQRRWFLDKLVDEIGVGYVYDSGSVSDYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 58 - protein, 163 aa, Artificial Sequence
  Description of Artificial Sequence Synthetic polypeptide
MNTKYNKEFLLYLAGFVDGDGSIIAQIKPNQSSKFKHDLSLTFKVTQKTQRRWFLDKLVDEIGVGYVYDSGSVSDYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 59 - protein, 163 aa, Artificial Sequence
  Description of Artificial Sequence Synthetic polypeptide
MNTKYNKEFLLYLAGFVDGDGSIIAQIKPNQTHKFKHQLSLTFKVTQKTQRRWFLDKLVDEIGVGYVYDSGSVSNYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 60 - protein, 163 aa, Artificial Sequence
  Description of Artificial Sequence Synthetic polypeptide
MNTKYNKEFLLYLAGFVDGDGSIIAQIKPNQSYKFKHQLSLTFRVTQKTQRRWFLDKLVDEIGVGYVYDSGSVSNYILSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 61 - DNA, 59 bp, Artificial Sequence
  Description of Artificial Sequence Synthetic oligonucleotide
gcataaattactatacttctatagacacgcaaacacaaatacacagcggccttgccacc

SEQ ID NO: 62 - DNA, 48 bp, Artificial Sequence
  Description of Artificial Sequence Synthetic oligonucleotide
ggctcgaggagctcgtctagaggatcgctcgagttatcagtcggccgc

SEQ ID NO: 63 - protein, 163 aa, Artificial Sequence
  Description of Artificial Sequence Synthetic polypeptide
MNTKYNKEFLLYLAGFVDGDGSIIAQIKPNQTHKFKHQLSLTFQVTQKTQRRWFLDKLVDEIGVGYVRDSGSVSHYYLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 64 - protein, 163 aa, Artificial Sequence
  Description of Artificial Sequence Synthetic polypeptide
MNTKYNKEFLLYLAGFVDGDGSIIAQIKPNQTHKFKHQLSLTFQVTQKTQRRWFLDKLVDEIGVGYVNDSGSVSQYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 65 - protein, 163 aa, Artificial Sequence
  Description of Artificial Sequence Synthetic polypeptide
MNTKYNKEFLLYLAGFVDGDGSIIAQIRPNQSSKFKHYLQLTFRVTQKTQRRWFLDKLVDEIGVGYVADSGSVSNYNLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 66 - protein, 163 aa, Artificial Sequence
  Description of Artificial Sequence Synthetic polypeptide
MNTKYNKEFLLYLAGFVDGDGSIIAQIRPNQSSKFKHYLQLTFRVTQKTQRRWFLDKLVDEIGVGYVADSGSVSHYYLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 67 - protein, 163 aa, Artificial Sequence
  Description of Artificial Sequence Synthetic polypeptide
MNTKYNKEFLLYLAGFVDGDGSIIAQIRPNQTSKFKHYLQLTFRVTQKTQRRWFLDKLVDEIGVGYVYDSGSVSNYNLSEIKPLHNFLTQLQPFLKLKQKQANLVLKIIEQLPSAKESPDKFLEVCTWVDQIAALNDSKMRKTTSETVRAVLDSLSEKKKSSP

SEQ ID NO: 68 - protein, 163 aa, Artificial Sequence
  Description of Artificial Sequence Synthetic polypeptide
MNTKYNKEFLLYLAGFVDGDGSIIAQIKPNQTHKFKHHLSLTFQVTQKTQRRWFLDKLVDEIGVGYVYDSGSVSDYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 69 - protein, 163 aa, Artificial Sequence
  Description of Artificial Sequence Synthetic polypeptide
MNTKYNKEFLLYLAGFVDGDGSIIAQIKPNQTHKFKHQLSLTFKVTQKTQRRWFLDKLVDEIGVGYVYDSGSVSDYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 70 - protein, 163 aa, Artificial Sequence
  Description of Artificial Sequence Synthetic polypeptide
MNTKYNKEFLLYLAGFVDGDGSIIAQIKPNQTHKFKHQLSLTFQVTQKTQRRWFLDKLVDEIGVGYVNDSGSVSHYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 71 - protein, 163 aa, Artificial Sequence
  Description of Artificial Sequence Synthetic polypeptide
MNTKYNKEFLLYLAGFVDGDGSIIAQIKPNQTHKFKHQLSLTFQVTQKTQRRWFLDKLVDEIGVGYVRDSGSVSHYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 72 - protein, 163 aa, Artificial Sequence
  Description of Artificial Sequence Synthetic polypeptide
MNTKYNKEFLLYLAGFVDGDGSIIAQIKPNQTHKFKHQLSLTFQVTQKTQRRWFLDKLVDEIGVGYVHDSGSVSHYHLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 73 - protein, 163 aa, Artificial Sequence
  Description of Artificial Sequence Synthetic polypeptide
MNTKYNKEFLLYLAGFVDGDGSIIAQIKPNQTHKFKHQLSLTFQVTQKTQRRWFLDKLVDEIGVGYVHDSGSVSHYHLSEIKPLHNFLTQLRPFLKLKQKQANLVLKIIEQLPSAKESPDKFLEVCTWVDQIAALNDSKTRKTTSETVRAVLDSLSEKKKSSP

SEQ ID NO: 74 - protein, 163 aa, Artificial Sequence
  Description of Artificial Sequence Synthetic polypeptide
MNTKYNKEFLLYLAGFVDGDGSIIAQIKPNQTHKFKHQLSLTFKVTQKTQRRWFLDKLVDEIGVGYVYDSGSVSDYRLSEIKPLHNFLTQLRPFLRLKQKQANLVLRIIEQLPSAKESPDKFLEVCTWVDQVAALNDSKARKATSETVRAVLDSLSEKKKSSP

SEQ ID NO: 75 - protein, 163 aa, Artificial Sequence
  Description of Artificial Sequence Synthetic polypeptide
MNTKYNKEFLLYLAGFVDGDGSIIAQIKPNQSHKFKHQLSLTFQVTQKTQRRWFLDKLVDEIGVGCVRDRGSVSDYILSEIKPLHNFLTQLQPFLKLKQKQANLVLKIIEQLPSAKESPDKFLEVCTWVDQIAALNVSKTRKTTSETVRAVLDSRSEKKKSPP

SEQ ID NO: 76 - protein, 163 aa, Artificial Sequence
  Description of Artificial Sequence Synthetic polypeptide
MNTKYNKELLLYLAGFVDGDGSIIAQIKPNQSHKFKHQLSLTFQVTQKTQRRWFLDKLVDEIGVGYVRDRGSVSDYILSEIKPLHNFLTQLQPFLKLKQIQANLVLKVIEQLPSAKESPDKFLEVCTWVDQIAALNDSKTRKTTSETVRAVLDGPSEKKKPSP

SEQ ID NO: 77 - protein, 163 aa, Artificial Sequence
  Description of Artificial Sequence Synthetic polypeptide
MYTKYNKEFLLYLAGFVDGDGSIIAQIKPNQSHKFKHQLSLTFQVTQKTQRRWFLDKLVDEIGVGYVRDRGSVSDYILSEIKPLHNFLTQLQPFLKLKQKQANLVLKIVEQLPSAKESPDKFLEACTWVDQIAALNDSKTRKTTSETVRAVLDSLSEKKKSSP

SEQ ID NO: 78 - protein, 163 aa, Artificial Sequence
  Description of Artificial Sequence Synthetic polypeptide
MNTKYNKEFLLYLAGFVDGDGSIIAQIKPNQSHKFKHQLSLTFQVTQKTQRRWFLDKLVDEIGVGYVRDRGSVSDYILSEIKPLHNFLTQLQPFLKLKQKQANLVLKIIEQLSSAKESPDKFLEVCTWVDQIAALSDSKTRKTTSETVRAVLDSLSEKKKSSP

SEQ ID NO: 79 - protein, 163 aa, Artificial Sequence
  Description of Artificial Sequence Synthetic polypeptide
MITKYNKEFLLYLAGFVDGDGSIIAQIKPNQSHKFKHQLSLTFQVTQKTQRRWFLDKLVDEIGVGYVRDRGSVSDYILSEVKPLHIFLTQLQPFLKLKQKQANLVLKIIGQLPSAKESPDKFLEVCTWVDRIAAQNDSKTRKTTSETVRAALDSLSVKKKSSP

SEQ ID NO: 80 - protein, 163 aa, Artificial Sequence
  Description of Artificial Sequence Synthetic polypeptide
MNTKYNKEFLLYLAGFVDGDGSIIAQIKPNQSHKFKHQLSLTFQVTQKTQRRWFLDKLVDEIGVGYVRDRRSVSDYILSEIKPLHNFLTQLQPFLKLKQKQAILVLKIIEQLPSAKESPDKFLEVCTWAGQIAALNDSKTRKTTSETVRAVLDSLSEKKKSSP

SEQ ID NO: 81 - protein, 163 aa, Artificial Sequence
  Description of Artificial Sequence Synthetic polypeptide
MNTKYNKEFLLYLAGFVDGDGSIIAQIKPNQSHKFKHQLSLTFQVTQKTQRRWFLDKLVDEIGVGYVRVRGSVSDYILSEIRPLHNFLTRLQPFLKLKQKQANLVLKIIEQLPSAKESPVKFLEVCTWVDQIAALNDSRTRKTTSETVRAVLDSLSEMKKSSP

SEQ ID NO: 82 - protein, 163 aa, Artificial Sequence
  Description of Artificial Sequence Synthetic polypeptide
MNTKYNKEFLLYLAGFVDGDGSIIAQIKPNQSHKFKHQLSLTFQVTQKTQRRWLLDKLVDEIGVGYVRDRGSVSDYILSEIKPLHDFLTQLQPFLKLKQRQANMALKIIEQLPSAKESPDKFLEVCTWVDQIAALSDSKTRKTTSETVRAVLDSLSEKRKSSP

SEQ ID NO: 83 - protein, 163 aa, Artificial Sequence
  Description of Artificial Sequence Synthetic polypeptide
MNTKYNKEFLLYLAGFVDGDGSIIAQIKPQQDYKFKHQLSLTFNVTQKTQRRWFLDKLVDEIGVGYVKDSGSVSRYTLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 84 - protein, 163 aa, Artificial Sequence
  Description of Artificial Sequence Synthetic polypeptide
MNTKYNKEFLLYLAGFVDGDGSIIAQIKPDQSRKFKHQLSLTFNVTQKTQRRWFLDKLVDEIGVGYVQDSGSVSSYVLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 85 - protein, 163 aa, Artificial Sequence
  Description of Artificial Sequence Synthetic polypeptide
MNTKYNKEFLLYLAGFVDGDGSIIAQIKPTQSGKFKHQLSLTFQVTQKTQRRWFLDKLVDEIGVGYVRDSGSVSRYYLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 86 - protein, 163 aa, Artificial Sequence
  Description of Artificial Sequence Synthetic polypeptide
MNTKYNKEFLLYLAGFVDGDGSIIAQIKPNQTYKFKHWLSLTFNVTQKTQRRWFLDKLVDEIGVGYVRDSGSVSYYILSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 87 - protein, 163 aa, Artificial Sequence
  Description of Artificial Sequence Synthetic polypeptide
MNTKYNKEFLLYLAGFVDGDGSIIAQIKPNQSTKFKHQLALTFNVTQKTQRRWFLDKLVDEIGVGYVKDSGSVSRYTLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 88 - protein, 163 aa, Artificial Sequence
  Description of Artificial Sequence Synthetic polypeptide
MNTKYNKEFLLYLAGFVDGDGSIIAQIKPNQSHKFKHSLSLTFRVTQKTQRRWFLDKLVDEIGVGYVRDSGSVSNYDLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 89 - protein, 163 aa, Artificial Sequence
  Description of Artificial Sequence Synthetic polypeptide
MNTKYNKEFLLYLAGFVDGDGSIIAQIKPNQSGKFKHTLSLTFNVTQKTQRRWFLDKLVDEIGVGYVKDSGSVSHYFLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 90 - protein, 163 aa, Artificial Sequence
  Description of Artificial Sequence Synthetic polypeptide
MNTKYNKEFLLYLAGFVDGDGSIIAQIKPKQSRKFKHQLSLTFDVTQKTQRRWFLDKLVDEIGVGYVYDSGSVSSYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 91 - protein, 163 aa, Artificial Sequence
  Description of Artificial Sequence Synthetic polypeptide
MNTKYNKEFLLYLAGFVDGDGSIIAQIKPNQSTKFKHQLTLTFRVTQKTQRRWFLDKLVDEIGVGYVYDSGSVSEYYLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 92 - protein, 163 aa, Artificial Sequence
  Description of Artificial Sequence Synthetic polypeptide
MNTKYNKEFLLYLAGFVDGDGSIIAQIKPNQTTKFKHQLSLTFYVTQKTQRRWFLDKLVDEIGVGYVRDSGSVSYYVLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 93 - protein, 163 aa, Artificial Sequence
  Description of Artificial Sequence Synthetic polypeptide
MNTKYNKEFLLYLAGFVDGDGSIIAQIKPYQTCKFKHQLSLTFQVTQKTQRRWFLDKLVDEIGVGYVRDSGSVSDYKLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 94 - protein, 163 aa, Artificial Sequence
  Description of Artificial Sequence Synthetic polypeptide
MNTKYNKEFLLYLAGFVDGDGSIIAQIKPNQSHKFKHGLSLTFNVTQKTQRRWFLDKLVDEIGVGYVRDSGSVSYYNLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 95 - protein, 163 aa, Artificial Sequence
  Description of Artificial Sequence Synthetic polypeptide
MNTKYNKEFLLYLAGFVDGDGSIIAQIKPNQSYKFKHQLSLTFAVTQKTQRRWFLDKLVDEIGVGYVRDSGSVSQYELSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 96 - protein, 163 aa, Artificial Sequence
  Description of Artificial Sequence Synthetic polypeptide
MNTKYNKEFLLYLAGFVDGDGSIIAQIKPRQSYKFKHELSLTFQVTQKTQRRWFLDKLVDEIGVGYVHDHGSVSNYILSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 97 - protein, 163 aa, Artificial Sequence
  Description of Artificial Sequence Synthetic polypeptide
MNTKYNKEFLLYLAGFVDGDGSIIAQIKPNQSGKFKHCLSLTFQVTQKTQRRWFLDKLVDEIGVGYVRDRGSVSNYILSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 98 - protein, 163 aa, Artificial Sequence
  Description of Artificial Sequence Synthetic polypeptide
MNTKYNKEFLLYLAGFVDGDGSIIAQIKPNQSTKFKHGLSLTFTVTQKTQRRWFLDKLVDEIGVGYVRDSGSVSYYTLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 99 - protein, 163 aa, Artificial Sequence
  Description of Artificial Sequence Synthetic polypeptide
MNTKYNKEFLLYLAGFVDGDGSIIAQIKPNQSTKFKHQLRLTFNVTQKTQRRWFLDKLVDEIGVGYVEDSGSVSRYKLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 100 - protein, 163 aa, Artificial Sequence
  Description of Artificial Sequence Synthetic polypeptide
MNTKYNKEFLLYLAGFVDGDGSIIAQIKPNQSHKFKHTLSLTFNVTQKTQRRWFLDKLVDEIGVGYVRDNGSVSNYILSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 101 - protein, 163 aa, Artificial Sequence
  Description of Artificial Sequence Synthetic polypeptide
MNTKYNKEFLLYLAGFVDGDGSIIAQIKPNQSGKFKHQLQLTFQVTQKTQRRWFLDKLVDEIGVGYVADKGSVSNYILSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 102 - protein, 163 aa, Artificial Sequence
  Description of Artificial Sequence Synthetic polypeptide
MNTKYNKEFLLYLAGFVDGDGSIIAQIKPCQSTKFKHQLSLTFQVTQKTQRRWFLDKLVDEIGVGYVYDSGSVSRYQLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 103 - protein, 163 aa, Artificial Sequence
  Description of Artificial Sequence Synthetic polypeptide
MNTKYNKEFLLYLAGFVDGDGSIIAQIKPNQSTKFKHQLQLTFNVTQKTQRRWFLDKLVDEIGVGYVKDSGSVSHYFLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 104 - protein, 163 aa, Artificial Sequence
  Description of Artificial Sequence Synthetic polypeptide
MNTKYNKEFLLYLAGFVDGDGSIIAQIKPNQSRKFKHTLSLTFRVTQKTQRRWFLDKLVDEIGVGYVTDSGSVSNYNLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 105 - protein, 163 aa, Artificial Sequence
  Description of Artificial Sequence Synthetic polypeptide
MNTKYNKEFLLYLAGFVDGDGSIIAQIKPNQGTKFKHQLSLTFNVTQKTQRRWFLDKLVDEIGVGYVYDSGSVSRYYLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 106 - protein, 163 aa, Artificial Sequence
  Description of Artificial Sequence Synthetic polypeptide
MNTKYNKEFLLYLAGFVDGDGSIIAQIKPNQWTKFKHQLSLTFQVTQKTQRRWFLDKLVDEIGVGYVRDRGSVSNYILSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 107 - protein, 163 aa, Artificial Sequence
  Description of Artificial Sequence Synthetic polypeptide
MNTKYNKEFLLYLAGFVDGDGSIIAQIKPNQSTKFKHALSLTFQVTQKTQRRWFLDKLVDEIGVGYVRDSGSVSNYLLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 108 - protein, 163 aa, Artificial Sequence
  Description of Artificial Sequence Synthetic polypeptide
MNTKYNKEFLLYLAGFVDGDGSIIAQIKPNQSCKFKHYLSLTFRVTQKTQRRWFLDKLVDEIGVGYVYDSGSVSDYILSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 109 - DNA, 49 bp, Artificial Sequence
  Description of Artificial Sequence Synthetic oligonucleotide
ggcatacaagtttcnnnacgtcgtacgacgtnnngacaatcgtctgtca

SEQ ID NO: 110 - DNA, 49 bp, Artificial Sequence
  Description of Artificial Sequence Synthetic oligonucleotide
ggcatacaagtttcaaaacnnngtacnnngttttgacaatcgtctgtca

SEQ ID NO: 111 - DNA, 501 bp, Artificial Sequence
  Description of Artificial Sequence Synthetic polynucleotide
atggccaataccaaatataacaaagagttcctgctgtacctggccggctttgtggacggtgacggtagcatcatcgctcagattaaaccaaaccagtcttataagtttaaacatcagctaagcttgacctttcaggtgactcaaaagacccagcgccgttggtttctggacaaactagtggatgaaattggcgttggttacgtacgtgatcgcggatccgtttccaactacatcttaagcgaaatcaagccgctgcacaacttcctgactcaactgcagccgtttctgaaactgaaacagaaacaggcaaacctggttctgaaaattatcgaacagctgccgtctgcaaaagaatccccggacaaattcctggaagtttgtacctgggtggatcagattgcagctctgaacgattctaagacgcgtaaaaccacttctgaaaccgttcgtgctgtgctggacagcctgagcgagaagaagaaatcctccccggcggccgac

SEQ ID NO: 112 - DNA, 68 bp, Artificial Sequence
  Description of Artificial Sequence Synthetic oligonucleotide
ctgaaaggtcaagcttagmbbatgtttaaacttmbbagactgmbbtggtttaatctgagcgatgatgc

SEQ ID NO: 113 - DNA, 68 bp, Artificial Sequence
  Description of Artificial Sequence Synthetic oligonucleotide
ctgaaaggtcaagcttagmbbatgtttaaacttataagactgmbbtggmbbaatctgagcgatgatgc

SEQ ID NO: 114 - DNA, 18 bp, Artificial Sequence
  Description of Artificial Sequence Synthetic oligonucleotide
ctaagcttgacctttcag

SEQ ID NO: 115 - DNA, 24 bp, Artificial Sequence
  Description of Artificial Sequence Synthetic oligonucleotide
tcaaaacggggtaccccgttttga

SEQ ID NO: 116 - DNA, 24 bp, Artificial Sequence
  Description of Artificial Sequence Synthetic oligonucleotide
tcaaaacggagtactccgttttga

SEQ ID NO: 117 - DNA, 24 bp, Artificial Sequence
  Description of Artificial Sequence Synthetic oligonucleotide
tcaaaacggtgtacaccgttttga

SEQ ID NO: 118 - DNA, 24 bp, Artificial Sequence
  Description of Artificial Sequence Synthetic oligonucleotide
tcaaaacggcgtacgccgttttga

SEQ ID NO: 119 - DNA, 24 bp, Artificial Sequence
  Description of Artificial Sequence Synthetic oligonucleotide
tcaaaacgaggtacctcgttttga

SEQ ID NO: 120 - DNA, 24 bp, Artificial Sequence
  Description of Artificial Sequence Synthetic oligonucleotide
tcaaaacgaagtacttcgttttga

SEQ ID NO: 121 - DNA, 24 bp, Artificial Sequence
  Description of Artificial Sequence Synthetic oligonucleotide
tcaaaacgatgtacatcgttttga

SEQ ID NO: 122 - DNA, 24 bp, Artificial Sequence
  Description of Artificial Sequence Synthetic oligonucleotide
tcgggacgtcgtacgacgtcccga

SEQ ID NO: 123 - DNA, 24 bp, Artificial Sequence
  Description of Artificial Sequence Synthetic oligonucleotide
tcggaacgtcgtacgacgttccga

SEQ ID NO: 124 - DNA, 24 bp, Artificial Sequence
  Description of Artificial Sequence Synthetic oligonucleotide
tcggtacgtcgtacgacgtaccga

SEQ ID NO: 125 - DNA, 24 bp, Artificial Sequence
  Description of Artificial Sequence Synthetic oligonucleotide
tcggcacgtcgtacgacgtgccga

SEQ ID NO: 126 - DNA, 24 bp, Artificial Sequence
  Description of Artificial Sequence Synthetic oligonucleotide
tcgagacgtcgtacgacgtctcga

SEQ ID NO: 127 - DNA, 24 bp, Artificial Sequence
  Description of Artificial Sequence Synthetic oligonucleotide
tcgaaacgtcgtacgacgtttcga

SEQ ID NO: 128 - DNA, 24 bp, Artificial Sequence
  Description of Artificial Sequence Synthetic oligonucleotide
tcgatacgtcgtacgacgtatcga

SEQ ID NO: 129 - protein, 163 aa, Artificial Sequence
  Description of Artificial Sequence Synthetic polypeptide
MNTKYNKEFLLYLAGFVDGDGSIIAQIKPSQSQKFKHQLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 130 - protein, 163 aa, Artificial Sequence
  Description of Artificial Sequence Synthetic polypeptide
MNTKYNKEFLLYLAGFVDGDGSIIAQIKPNQTHKFKHQLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 131 - protein, 163 aa, Artificial Sequence
  Description of Artificial Sequence Synthetic polypeptide
MNTKYNKEFLLYLAGFVDGDGSIIAQIKPNQSTKFKHYLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 132 - protein, 163 aa, Artificial Sequence; identical to SEQ ID NO: 27
  Description of Artificial Sequence Synthetic polypeptide

SEQ ID NO: 133 - protein, 163 aa, Artificial Sequence; identical to SEQ ID NO: 38
  Description of Artificial Sequence Synthetic polypeptide

SEQ ID NO: 134 - protein, 163 aa, Artificial Sequence
  Description of Artificial Sequence Synthetic polypeptide
MNTKYNKEFLLYLAGFVDGDGSIIAQIKPNQSYKFKHQLSLTFRVTQKTQRRWFLDKLVDEIGVGYVADSGSVSSYNLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 135 - protein, 163 aa, Artificial Sequence
  Description of Artificial Sequence Synthetic polypeptide
MNTKYNKEFLLYLAGFVDGDGSIIAQIKPNQSYKFKHQLSLTFRVTQKTQRRWFLDKLVDEIGVGYVYDSGSVSDYNLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 136 - protein, 163 aa, Artificial Sequence
  Description of Artificial Sequence Synthetic polypeptide
MNTKYNKEFLLYLAGFVDGDGSIIAQIKPNQSYKFKHQLSLTFRVTQKTQRRWFLDKLVDEIGVGYVYDSGSVSDYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 137 - protein, 163 aa, Artificial Sequence
  Description of Artificial Sequence Synthetic polypeptide
MNTKYNKEFLLYLAGFVDGDGSIIAQIKPNQSYKFKHQLSLTFQVTQKTQRRWFLDKLVDEIGVGYVNDSGSVSQYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 138 - protein, 163 aa, Artificial Sequence
  Description of Artificial Sequence Synthetic polypeptide
MNTKYNKEFLLYLAGFVDGDGSIIAQIKPNQSYKFKHQLSLTFQVTQKTQRRWFLDKLVDEIGVGYVRDSGSVSHYYLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 139 - protein, 163 aa, Artificial Sequence
  Description of Artificial Sequence Synthetic polypeptide
MNTKYNKEFLLYLAGFVDGDGSIIAQIKPNQSYKFKHQLSLTFKVTQKTQRRWFLDKLVDEIGVGYVYDSGSVSEYVLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 140 - protein, 163 aa, Artificial Sequence
  Description of Artificial Sequence Synthetic polypeptide
MNTKYNKEFLLYLAGFVDGDGSIIAQIKPNQSYKFKHQLSLTFKVTQKTQRRWFLDKLVDEIGVGYVYDSGSVSDYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 141 - protein, 163 aa, Artificial Sequence
  Description of Artificial Sequence Synthetic polypeptide
MNTKYNKEFLLYLAGFVDGDGSIIAQIKPNQSYKFKHQLSLTFKVTQKTQRRWFLDKLVDEIGVGYVYDSGSVSNYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 142 - protein, 163 aa, Artificial Sequence; identical to SEQ ID NO: 60
  Description of Artificial Sequence Synthetic polypeptide

SEQ ID NO: 143 - protein, 163 aa, Chlamydomonas reinhardtii
MNTKYNKEFLLYLAGFVDGDGSIIAQIKPNQSYKFKHQLSLAFQVTQKTQRRWFLDKLVDEIGVGYVRDRGSVSDYILSEIKPLHNFLTQLQPFLKLKQKQANLVLKIIWRLPSAKESPDKFLEVCTWVDQIAALNDSKTRKTTSETVRAVLDSLSEKKKSSP

SEQ ID NO: 144 - protein, 163 aa, Chlamydomonas reinhardtii
MNTKYNKEFLLYLAGFVDGDGSIIAQIKPNQSYKFKHQLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 145 - DNA, 1289 bp, Mus musculus
ttacctcactgctttccggagcggtagcacctcctccgccggcttcctcctcagaccgctttttgccgcgagccgaccggtcccgtcatgccgacccgcagtcccagcgtcgtgattagcgatgatgaaccaggttatgacctagatttgttttgtatacctaatcattatgccgaggatttggaaaaagtgtttattcctcatggactgattatggacaggactgaaagacttgctcgagatgtcatgaaggagatgggaggccatcacattgtggccctctgtgtgctcaaggggggctataagttctttgctgacctgctggattacattaaagcactgaatagaaatagtgatagatccattcctatgactgtagattttatcagactgaagagctactgtaatgatcagtcaacgggggacataaaagttattggtggagatgatctctcaactttaactggaaagaatgtcttgattgttgaagatataattgacactggtaaaacaatgcaaactttgctttccctggttaagcagtacagccccaaaatggttaaggttgcaagcttgctggtgaaaaggacctctcgaagtgttggatacaggccagactttgttggatttgaaattccagacaagtttgttgttggatatgcccttgactataatgagtacttcaggaatttgaatcacgtttgtgtcattagtgaaactggaaaagccaaatacaaagcctaagatgagcgcaagttgaatctgcaaatacgaggagtcctgttgatgttgccagtaaaattagcaggtgttctagtcctgtggccatctgcctagtaaagctttttgcatgaaccttctatgaatgttactgttttatttttagaaatgtcagttgctgcgtccccagacttttgatttgcactatgagcctataggccagcctaccctctggtagattgtcgcttatcttgtaagaaaaacaaatctcttaaattaccacttttaaataataatactgagattgtatctgtaagaaggatttaaagagaagctatattagttttttaattggtattttaatttttatatattcaggagagaaagatgtgattgatattgttaatttagacgagtctgaagctctcgatttcctatcagtaacagcatctaagaggttttgctcagtggaataaacatgtttcagcagtgttggctgtattttcccactttcagtaaatcgttgtcaacagttccttttaaatgcaaataaataaattctaaaaatt

SEQ ID NO: 146 - DNA, 1331 bp, Artificial Sequence
  Description of Artificial Sequence Synthetic polynucleotide
tcttgctgcgcctccgcctcctcctctgctccgccaccggcttcctcctcctgagcagtcagcccgcgcgccggccggctccgttatggcgacccgcagccctggcgtcgtgattagtgatgatgaaccaggttatgaccttgatttattttgcatacctaatcattatgctgaggatttggaaagggtgtttattcctcatggactaattatggacaggactgaacgtcttgctcgagatgtgatgaaggagatgggaggccatcacattgtagccctctgtgtgctcaaggggggctataaattctttgctgacctgctggattacatcaaagcactgaatagaaatagtgatagatccattcctatgactgtagattttatcagactgaagagctattgtaatgaccagtcaacaggggacataaaagtaattggtggagatgatctctcaactttaactggaaagaatgtcttgattgtggaagatataattgacactggcaaaacaatgcagactttgctttccttggtcaggcagtataatccaaagatggtcaaggtcgcaagcttgctggtgaaaaggaccccacgaagtgttggatataagccagactttgttggatttgaaattccagacaagtttgttgtaggatatgcccttgactataatgaatacttcagggatttgaatcatgtttgtgtcattagtgaaactggaaaagcaaaatacaaagcctaagatgagagttcaagttgagtttggaaacatctggagtcctattgacatcgccagtaaaattatcaatgttctagttctgtggccatctgcttagtagagctttttgcatgtatcttctaagaattttatctgttttgtactttagaaatgtcagttgctgcattcctaaactgtttatttgcactatgagcctatagactatcagttccctttgggcggattgttgtttaacttgtaaatgaaaaaattctcttaaaccacagcactattgagtgaaacattgaactcatatctgtaagaaataaagagaagatatattagttttttaattggtattttaatttttatatatgcaggaaagaatagaagtgattgaatattgttaattataccaccgtgtgttagaaaagtaagaagcagtcaattttcacatcaaagacagcatctaagaagttttgttctgtcctggaattattttagtagtgtttcagtaatgttgactgtattttccaacttgttcaaattattaccagtgaatctttgtcagcagttcccttttaaatgcaaatcaataaattcccaaaaattt

SEQ ID NO: 147 - protein, 163 aa, Artificial Sequence
  Description of Artificial Sequence Synthetic polypeptide
MNTKYNKEFLLYLAGFVDGDGSIIAQIKPNQSHKFKHQLSLALQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 148 - protein, 163 aa, Artificial Sequence
  Description of Artificial Sequence Synthetic polypeptide
MNTKYNKEFLLYLAGFVDGDGSIIAQIKPNQTHKFKHQLSLTFRVTQKTQRRWFLDKLVDEIGVGYVYDSGTVSNYNLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 149 - DNA, 84 bp, Artificial Sequence
  Description of Artificial Sequence Synthetic oligonucleotide
aagcagagctctctggctaactagagaacccactgcttactggcttatcgaccatggccaataccaaatataacaaagagttcc

SEQ ID NO: 150 - DNA, 69 bp, Artificial Sequence
  Description of Artificial Sequence Synthetic oligonucleotide
tctgatcgattcaagtcagtgtctctctagatagcgagtcggccgccggggaggatttcttcttctcgc

SEQ ID NO: 151 - DNA, 33 bp, Artificial Sequence
  Description of Artificial Sequence Synthetic oligonucleotide
gccggctttgtggactctgacggtagcatcatc

SEQ ID NO: 152 - DNA, 33 bp, Artificial Sequence
  Description of Artificial Sequence Synthetic oligonucleotide
gatgatgctaccgtcagagtccacaaagccggc

SEQ ID NO: 153 - protein, 9 aa, Artificial Sequence
  Description of Artificial Sequence Synthetic peptide
LAGLIDADG

(19) United States
(12) Patent Application Publication
(10) Pub. No.: US 2010/0146651 A1
(43) Pub. Date: Jun. 10, 2010

(54) MEGANUCLEASE VARIANTS CLEAVING A DNA TARGET SEQUENCE FROM THE HPRT GENE AND USES THEREOF

(75) Inventors: Julianne Smith, Le Plessis Robinson (FR); Sylvestre Grizot, La Garenne Colombes (FR); Agnes Gouble, Paris (FR)

Correspondence Address: OBLON, SPIVAK, MCCLELLAND MAIER & NEUSTADT, L.L.P., 1940 DUKE STREET, ALEXANDRIA, VA, 22314, US

(73) Assignee: CELLECTIS, Romainville (FR)

(21) Appl. No.: 12/514,913

(22) PCT Filed: Nov. 13, 2007
(86) PCT No.: PCT/IB07/04281

§ 371 Date: Aug. 11, 2009

(30) Foreign Application Priority Data
Nov. 14, 2006 (IB) PCT/IB2006/004084
Jun. 25, 2007 (IB) PCT/IB2007/002881

(51) Int. Cl. A01K 67/033; C07H 21/00; C12N 15/63; C12N 9/16; C12N 5/00; A01H 5/00

(52) U.S. Cl. 800/13; 536/23.2; 435/320.1; 435/196; 435/325; 800/298

(57) ABSTRACT

An I-CreI variant or a single-chain derivative having at least one substitution in one of the two functional subdomains of the LAGLIDADG (SEQ ID NO: 153) core domain situated from positions 26 to 40 and 44 to 77 of I-CreI, and being able to cleave a DNA target sequence from the HPRT gene having a nucleotide sequence of SEQ ID NO: 1 to 14. Use of said variant for inducing a site-specific modification in the HPRT gene, for therapeutic (gene therapy of Lesch-Nyhan syndrome) or non-therapeutic purpose (engineering of transgenic animals and recombinant cell lines).

The invention relates to a meganuclease variant cleaving a DNA target sequence from the HPRT gene, to a vector encoding said variant, to a cell, an animal or a plant modified by said vector and to the use of said meganuclease variant and derived products for genome engineering and genome therapy.

Meganucleases are by definition sequence-specific endonucleases with large (>14 bp) cleavage sites that can deliver DNA double-strand breaks (DSBs) at specific loci in living cells (Thierry and Dujon, Nucleic Acids Res., 1992, 20, 5625-5631). Meganucleases have been used to stimulate homologous recombination in the vicinity of their target sequences in cultured cells and plants (Rouet et al., Mol. Cell. Biol., 1994, 14, 8096-106; Choulika et al., Mol. Cell. Biol., 1995, 15, 1968-73; Donoho et al., Mol. Cell. Biol, 1998, 18, 4070-8; Elliott et al., Mol. Cell. Biol., 1998, 18, 93-101; Sargent et al., Mol. Cell. Biol., 1997, 17, 267-77; Puchta et al., Proc. Natl. Acad. Sci. USA, 1996, 93, 5055-60; Chiurazzi et al., Plant Cell, 1996, 8, 2057-2066), making meganuclease-induced recombination an efficient and robust method for genome engineering.

The use of meganuclease-induced recombination has long been limited by the repertoire of natural meganucleases, and the major limitation of the current technology is the requirement for the prior introduction of a meganuclease cleavage site in the locus of interest. Thus, the making of artificial meganucleases with tailored substrate specificities is under intense investigation. Such proteins could be used to cleave genuine chromosomal sequences and open new perspectives for genome engineering in wide range of applications. For example, meganucleases could be used to knock out endogenous genes or knock-in exogenous sequences in the chromosome. It can as well be used for gene correction, and in principle, for the correction of mutations linked with monogenic diseases.

In nature, meganucleases are essentially represented by homing endonucleases (HEs), a family of endonucleases encoded by mobile genetic elements, whose function is to initiate DNA double-strand break (DSB)-induced recombination events in a process referred to as homing (Chevalier and Stoddard, Nucleic Acids Res., 2001, 29, 3757-74; Kostriken et al., Cell; 1983, 35, 167-74; Jacquier and Dujon, Cell, 1985, 41, 383-94). Several hundreds of HEs have been identified in bacteria, eukaryotes, and archea (Chevalier and Stoddard, precited); however the probability of finding a HE cleavage site in a chosen gene is very low.

Given their biological function and their exceptional cleavage properties in terms of efficacy and specificity, HEs provide ideal scaffolds to derive novel endonucleases for genome engineering.

HEs belong to four major families. The LAGLIDADG family, named after a conserved peptidic motif involved in the catalytic center, is the most widespread and the best characterized group (Chevalier and Stoddard, precited). Seven structures are now available. Whereas most proteins from this family are monomeric and display two LAGLIDADG motifs, a few ones have only one motif, but dimerize to cleave palindromic or pseudo-palidromic target sequences.

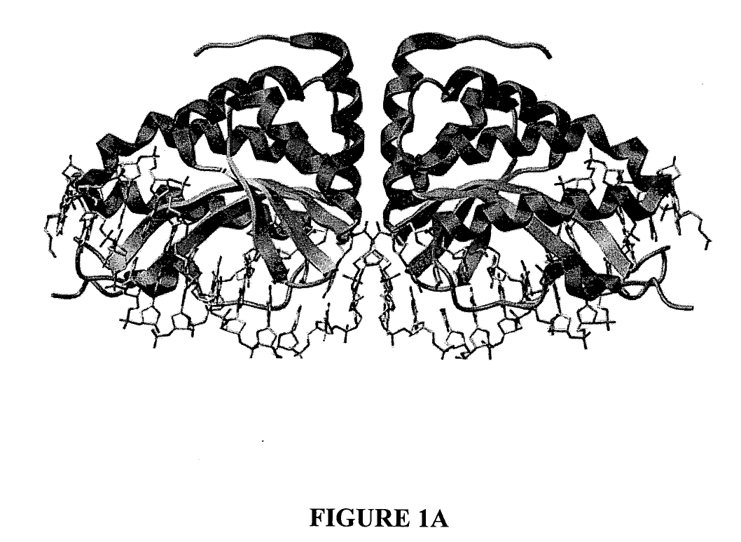

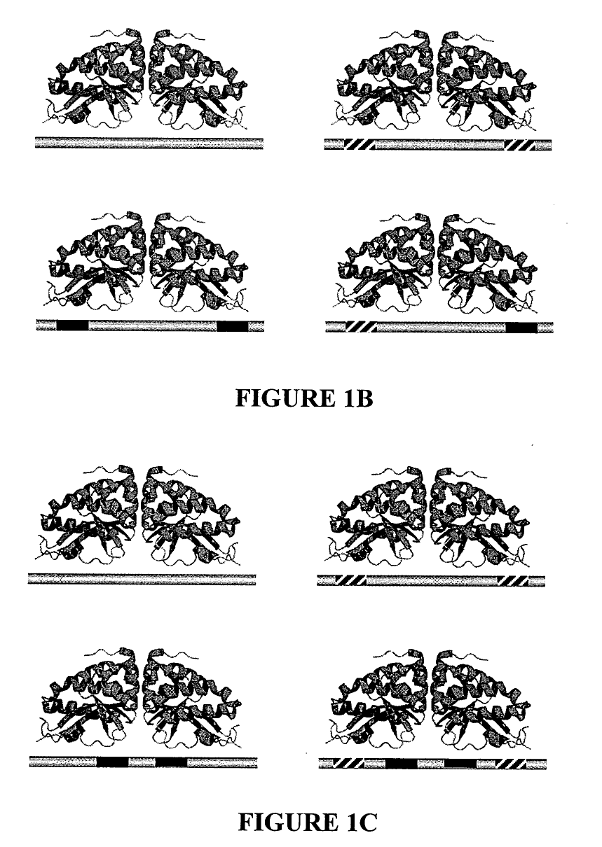

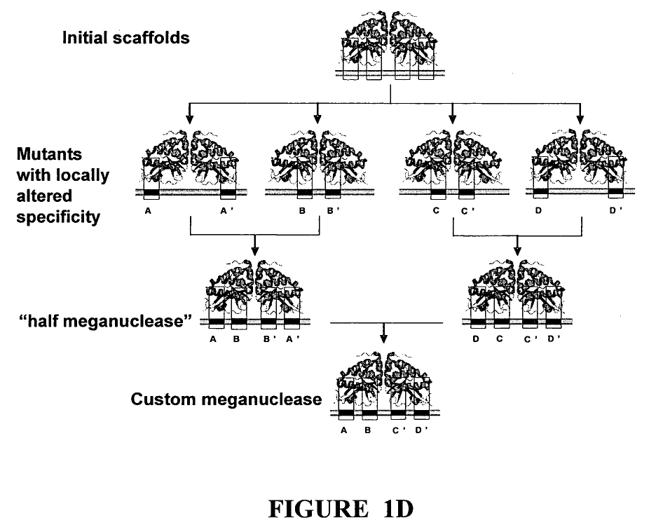

Although the LAGLIDADG peptide is the only conserved region among members of the family, these proteins share a very similar architecture (FIG. 1A). The catalytic core is flanked by two DNA-binding domains with a perfect two-fold symmetry for homodimers such as I-CreI (Chevalier et al., Nat. Struct. Biol., 2001, 8, 312-6) and I-MsoI (Chevalier et al. J. Mol. Biol., 2003, 329, 253-69), and with a pseudo symmetry fo monomers such as I-SceI (Moure et al., J. Mol. Biol, 2003, 334, 685-95), I-DmoI (Silva et al., J. Mol. Biol., 1999, 286, 1123-36) or I-AniI (Bolduc et al., Genes Dev., 2003, 17, 2875-88). Both monomers or both domains (for monomeric proteins) contribute to the catalytic core, organized around divalent cations. Just above the catalytic core, the two LAGLIDADG peptides play also an essential role in the dimerization interface. DNA binding depends on two typical saddle-shaped ββαββ folds, sitting on the DNA major groove. Analysis of I-CreI structure bound to its natural target shows that in each monomer, eight residues (Y33, Q38, N30, K28, Q26, Q44, R68 and R70) establish direct interaction with seven bases at positions ±3, 4, 5, 6, 7, 9 and 10 (Jurica et al., Mol. Cell., 1998, 2, 469-76). In addition, some residues establish water-mediated contact with several bases; for example S40, K28 and N30 with the base pair at position +8 and −8 (Chevalier et al., 2003, precited). Other domains can be found, for example in inteins such as PI-PfuI (Ichiyanagi et al., J. Mol. Biol., 2000, 300, 889-901) and PI-SceI (Moure et al., Nat. Struct. Biol., 2002, 9, 764-70), which protein splicing domain is also involved in DNA binding.

The making of functional chimeric and single chain artificial HEs, by fusing the N-terminal I-DmoI domain with an I-CreI monomer (Epinat et al., Nucleic Acids Res., 2003, 31, 2952-62; Chevalier et al., Mol. Cell., 2002, 10, 895-905; Steuer et al., Chembiochem., 2004, 5, 206-13; International PCT Applications WO 03/078619 and WO 2004/031346) have demonstrasted the plasticity of LAGLIDADG proteins: different monomers or core domains could be fused in a single protein, to obtain novel meganucleases cleaving novel (non-palindromic) target sequences.

Besides, different groups have have used a rational approach to locally alter the specificity of the I-CreI (Seligman et al., Genetics, 1997, 147, 1653-64; Sussman et al., J. Mol. Biol., 2004, 342, 31-41; Seligman et al., Nucleic Acids Res., published Sep. 13, 2006; Arnould et al., J. Mol. Biol., 2006, 355, 443-458 and International PCT Applications WO 2006/097853 and WO 2006/097784), I-SceI (Doyon et al., J. Am. Chem. Soc., 2006, 128, 24-77-2484), PI-SceI (Gimble et al., J. Mol. Biol., 2003, 334, 993-1008) and I-MsoI (Ashworth et al., Nature, 2006, 441, 656-659)

Hundreds of I-CreI derivatives with altered specificity were engineered by combining the semi-rational approach and High Throughput Screening (HTS; Arnould et al. (precited); International PCT Applications WO 2006/097853 and WO 2006/097784); residues Q44, R68 and R70 or Q44, R68, D75 and 177 of I-CreI were mutagenized and a collection of variants with altered specificity in positions ±3 to 5 (5NNN DNA target) were identified by screening.

Then, two different variants (FIG. 1B; top right and bottom left) were combined and assembled in a functional heterodimeric endonuclease (FIG. 1B; bottom right) able to cleave a chimeric target resulting from the fusion of a different half of each variant DNA target sequence (FIG. 1B; Arnould et al., precited; International PCT Application WO 2006/097854). Interestingly, the novel proteins had kept proper folding and stability, high activity, and a narrow specificity.

Therefore, a two step strategy may be used to tailor the specificity of a natural LAGLIDADG meganuclease. The first step is to locally mutagenize a natural LAGLIDADG meganuclease such as I-CreI and to identify collections of variants with altered specificity by screening. The second step is to rely on the modularity of these proteins, and use a combinatorial approach to make novel meganucleases, that cleave the site of choice (FIG. 1B).

The generation of collections of novel meganucleases, and the ability to combine them by assembling two different monomers/core domains considerably enriches the number of DNA sequences that can be targeted, but does not yet saturate all potential sequences.

To reach a larger number of sequences, it would be extremely valuable to be able to identify smaller independent subdomains that could be combined (FIG. 1C).

However, a combinatorial approach is much more difficult to apply within a single monomer or domain than between monomers since the structure of the binding interface is very compact and the two different ββ hairpins which are responsible for virtually all base-specific interactions do not constitute separate subdomains, but are part of a single fold. For example, in the internal part of the DNA binding regions of I-CreI, the gtc triplet is bound by one residue from the first hairpin (Q44), and two residues from the second hairpin (R68 and R70; see FIG. 1B of Chevalier et al., 2003, precited). In addition the cumulative impact of a series of mutation could eventually disrupt proper folding.

In spite of this lack of apparent modularity at the structural level, the Inventor has demonstrated that residues 28 to 40 and 44 to 77 of I-CreI form two separable functional subdomains, able to bind distinct parts of a homing endonuclease half-site.

By assembling two subdomains from different monomers or core domains within the same monomer, the Inventor has engineered functional homing endonuclease (homodimeric) variants, which are able to cleave palindromic chimeric targets (FIG. 1C). Furthermore, a larger combinatorial approach is allowed by assembling four different subdomains to form new heterodimeric molecules which are able to cleave non-palindromic chimeric targets (FIG. 1D). The different subdomains can be modified separately to engineer new cleavage specificities and combine in one meganuclease variant (homodimer, heterodimer, single-chain molecule) which is able to cleave a target from a gene of interest.

The Hypoxanthine Phosphoribosyltransferase (HPRT) gene is a single copy gene located on X-chromosome and thus present in one copy (XY cells) or expressed from just one allele (XX cells). For example, the mouse and human

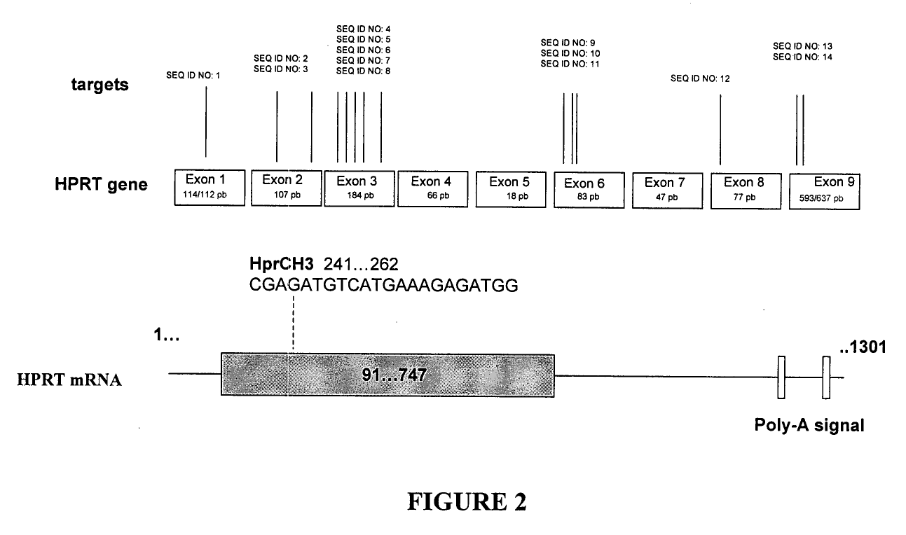

HPRT genes are available in the NCBI database, under the accession number NC_{—}000086 and NC_{—}000023, respectively. Both genes have 9 exons (FIG. 2) which are transcribed into a 1289 bases mRNA (mouse; accession number NM_{—}013556) or 1331 bases mRNA (human; accession number NM_{—}000194), containing the HPRT ORF from positions 88 to 744 (mouse) or 86 to 742 (human). The Chinese Hamster (Criteculus sp.) mRNA is a 1301 bases sequence (accession number J00060.1) containing the HPRT ORF from positions 91 to 747.

Hypoxanthine Phosphoribosyltransferase is an enzyme that catalyzes the conversion of 5-phosphoribosyl-1-pyrophosphate and hypoxanthine, guanine, or 6-mercaptopurine to the corresponding 5′-mononucleotides and pyrophosphate. The enzyme is important in purine biosynthesis as well as central nervous system function. Given its biological function, the HPRT gene is used as a selectable marker for gene targeting experiments. Compared to other selection markers, HPRT has the advantage of being both a positive and a negative selection marker. In addition mutations in the HPRT gene are associated with the Lesch-Nyhan syndrome.

Gene targeting of the mouse HPRT was performed first in Embryo-derived Stem (ES) cells by Thomas, K. R. and M. R. Cappechi (Cell, 1987, 51, 503-512). However, efficiencies remained very low (about 10⁻⁷ of transfected cells). The ability to generate a double-strand break at the HPRT locus provides a means to significantly enhance homologous recombination at the locus. Using classical gene targeting, Donoho et al. (Mol. Cell. Biol., 1998, 18, 4070-4078) introduced cleavage sites for the I-SceI meganuclease into the mouse HPRT gene. In a second step, they could induce gene targeting in 1% of the cells by cotransformation with a repair matrix and an I-SceI expression vector.

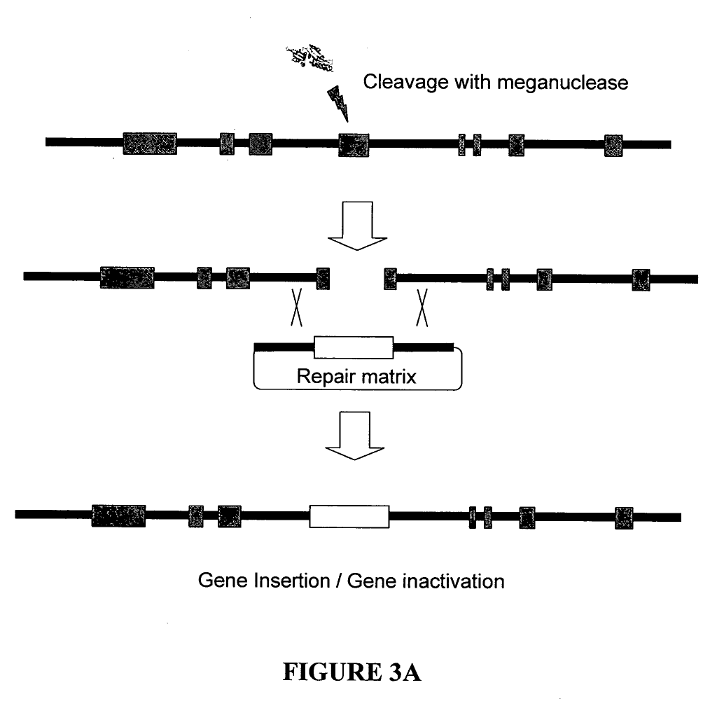

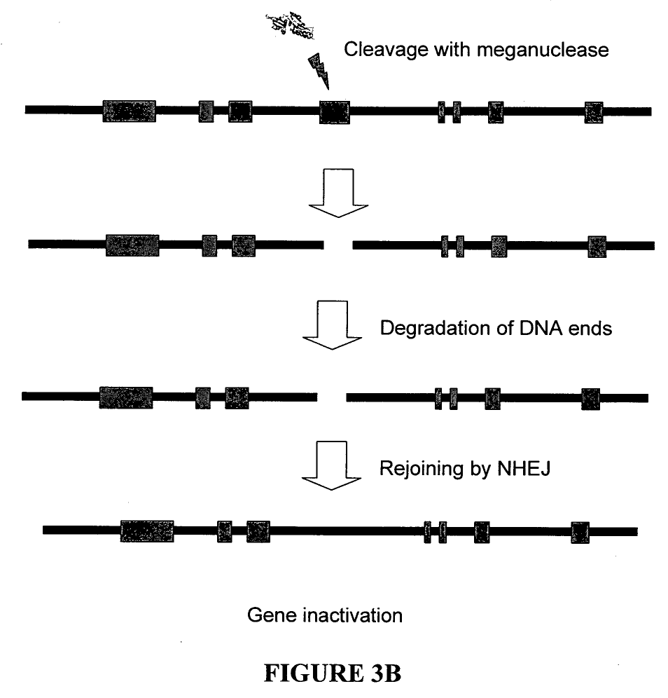

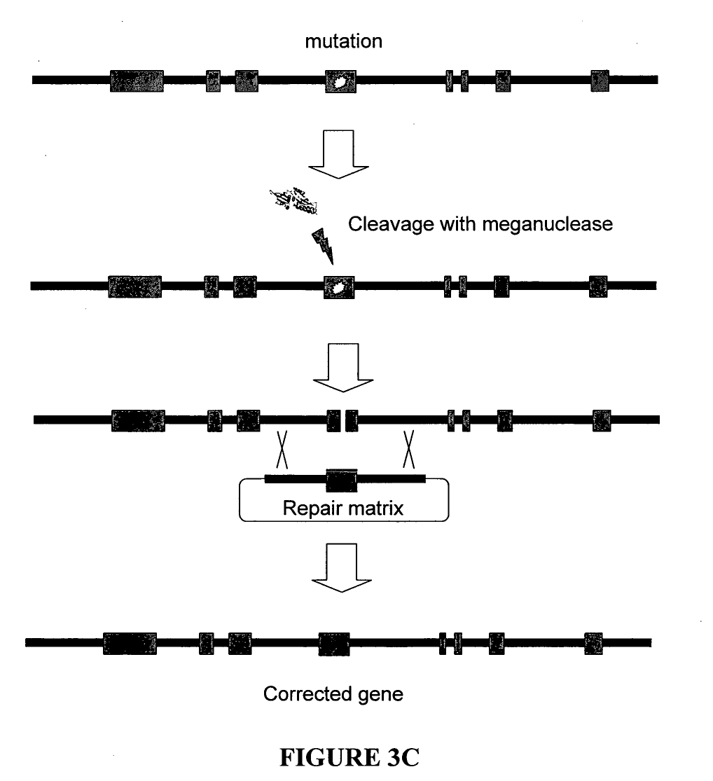

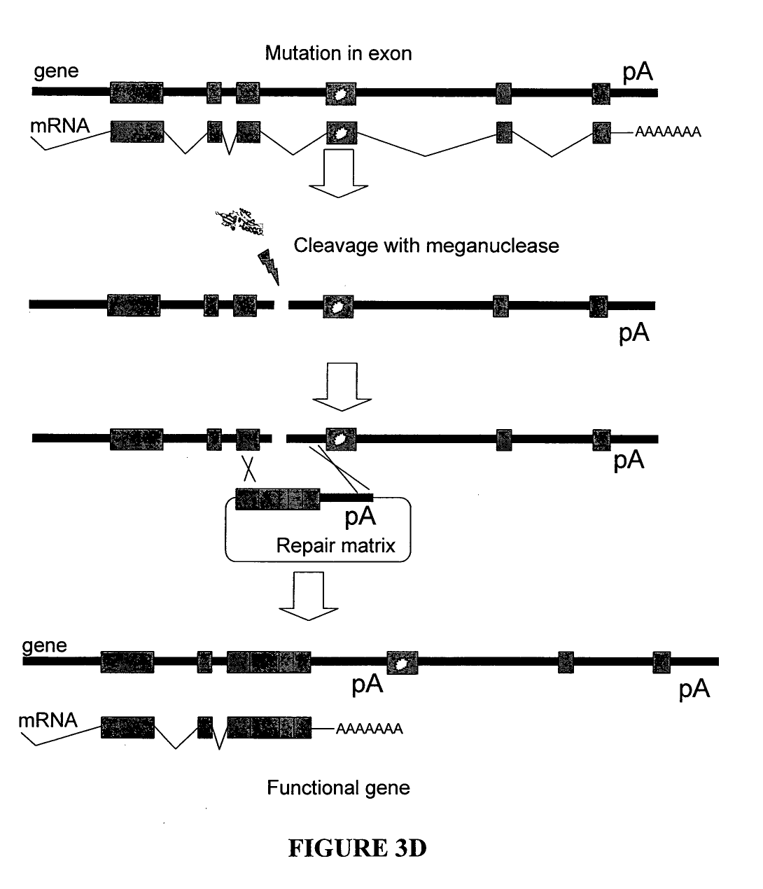

Thus, an artificial meganuclease targeting the HPRT locus will allow efficient gene insertions (FIG. 3A). The ability to efficiently insert genes at this locus has the advantage of allowing reproducible expression levels as well as predictable time lines for generating insertions.

Additionally, as has been described for mice (van der Lugt et al. Gene, 1991, 105, 263-267; Selfridge et al., Somat. Cell. Mol. Genet., 1992, 18, 325-336), HPRT can be used as a selectable marker for gene targeting experiments.

The double replacement gene targeting procedure, which was originally suggested by Reid and co-workers (Proc. Natl. Acad. Sci. USA, 1990, 87, 4299-4303) is based on HPRT selectable markers (Magin et al., Gene, 1992, 122, 289-296), to produce mice with subtle gene alterations. This procedure is based on the use of hypoxanthine phosphoribosyltransferase (HPRT) minigenes in HPRT-deficient embryonic stem cells and the ability to select both for and against HPRT expression.

In the first step, to inactivate the target, a region of the target locus is replaced with an HPRT minigene, with HAT (hypoxanthine/aminopterin/thymidine; (Littlefield J. W., Science, 1964, 145, 709-) selection for HPRT marker expression. HAT is a mixture of sodium hypoxanthine, aminopterin and thymidine. Aminopterin is a potent folic acid antagonist, which inhibits dihydrofolate reductase blocking de novo nucleoside synthesis. Cells can only survive in HAT if the purine and pyrimidine salvage pathways are active. Hypoxanthine is the substrate for purine salvage pathway. Thus, HPRT mutants are unable to utilize the purine salvage pathway and are sensitive to HAT selection.

In the second targeting step the HPRT minigene is itself replaced with an altered region of the target gene to reconstitute the locus, with selection for loss of the HPRT marker using the purine analogue 6-thioguanine (6-TG).

However, this requires that the cells before introduction of the marker contain an inactive HPRT gene. Thus, an artificial meganuclease targeting the HPRT gene could be used to inactivate the HPRT gene (FIGS. 3A and B).

The Lesch-Nyhan syndrome is an inherited disorder transmitted as a sex-linked trait that is caused by a deficiency of HPRT and characterized by hyperuricemia, severe motor disability and self-injurious behaviour.

A very heterogeneous collection of mutations associated with the Lesch-Nyhan disease or less severe clinical phenotypes with only some portions of the full syndrome, have been identified. Current gene therapy strategies are based on a complementation approach, wherein randomly inserted but functional extra copy of the gene provide for the function of the mutated endogenous copy. In contrast, meganuclease-induced recombination should allow for the precise correction of mutations in situ (FIG. 3C) and thereby bypass the risk due to the randomly inserted transgenes encountered with current gene therapy approaches (Hacein-Bey-Abina et al., Science, 2003, 302, 415-419).

The most accurate way to correct a genetic defect is to use a repair matrix with a non mutated copy of the gene, resulting in a reversion of the mutation (FIG. 3C). However, the efficiency of gene correction decreases as the distance between the mutation and the DSB grows, with a five-fold decrease by 200 by of distance. Therefore, a given meganuclease can be used to correct only mutations in the vicinity of its DNA target. An alternative, termed “exon knock-in” is featured in FIG. 3D. In this case, a meganuclease cleaving in the 5′ part of the gene can be used to knock-in functional exonic sequences upstream of the deleterious mutation. Although this method places the transgene in its regular location, it also results in exons duplication, which impact on the long range remains to be evaluated. In addition, should naturally cis-acting elements be placed in an intron downstream of the cleavage, their immediate environment would be modified and their proper function would also need to be explored. However, this method has a tremendous advantage: a single meganuclease could be used for many different patients.

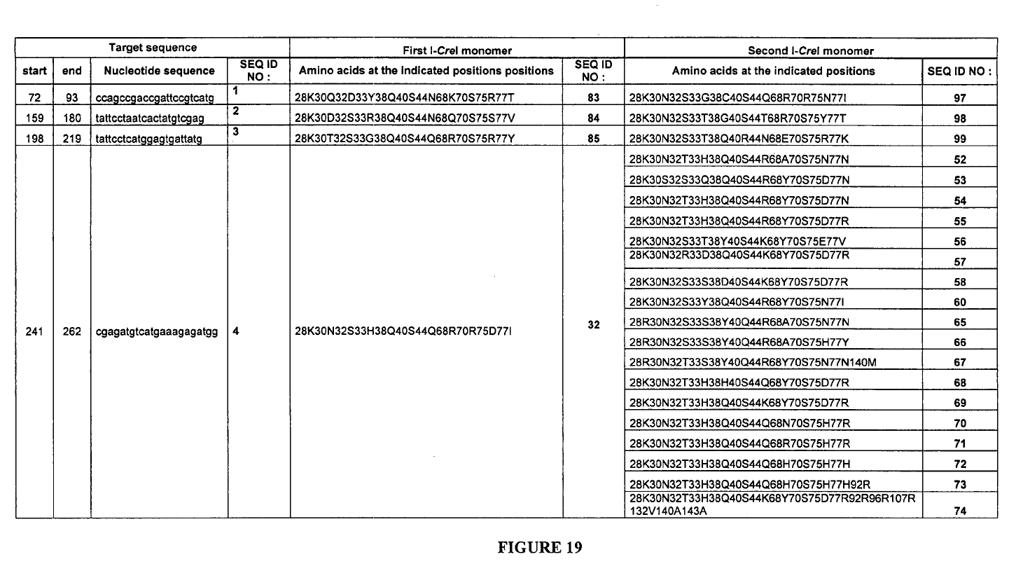

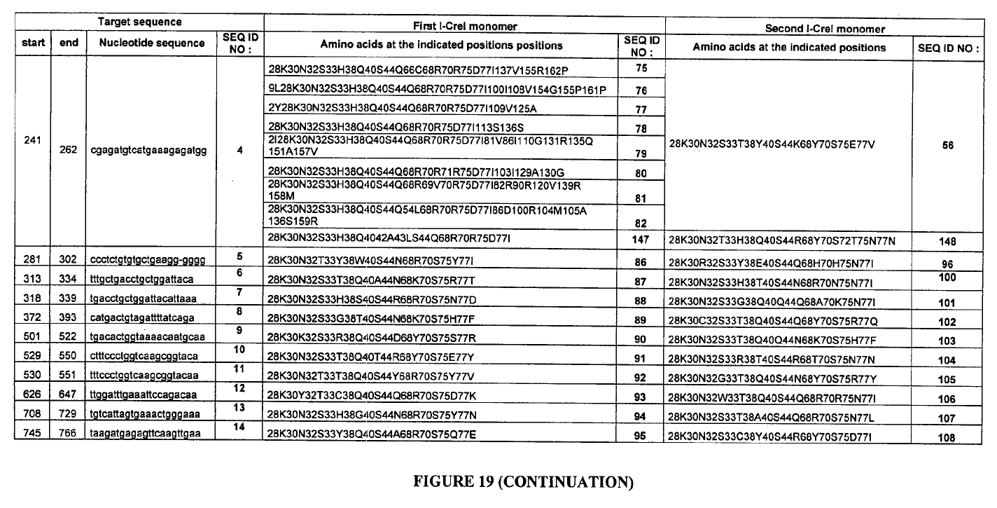

The Inventor has identified a series of DNA targets in the HPRT gene that could be cleaved by I-CreI variants (FIGS. 2 and 19). The combinatorial approach described in FIG. 1D was used to assemble four set of mutations into heterodimeric homing endonucleases with fully engineered specificity, to cleave the DNA targets from the HPRT gene. These I-CreI variants which are able to cleave a genomic DNA target from the HPRT gene can be used for genome engineering at the HPRT locus (knock-out and knock in) and for using HPRT as a selectable marker for genome engineering at any locus (FIGS. 3A and 3B).

In addition, these meganucleases could be used for repairing the HPRT mutations associated with the Lesch-Nyhan syndrome (FIGS. 3C and 3D).

The invention relates to the use of an I-CreI variant or a single-chain derivative for inducing a site-specific modification in the HPRT gene, for non-therapeutic purpose, wherein said I-CreI variant or single-chain derivative has at least one substitution in one of the two functional subdomains of the LAGLIDADG core domain situated from positions 26 to 40 and 44 to 77 of I-CreI, and is able to cleave a DNA target sequence selected from the group consisting of the sequences SEQ ID NO: 1 to 14.

The cleavage activity of the variant as defined in the present invention may be measured by any well-known, in vitro or in vivo cleavage assay, such as those described in the International PCT Application WO 2004/067736, Arnould et al. (J. Mol. Biol., 2006, 355, 443-458), Epinat et al. (Nucleic Acids Res., 2003, 31, 2952-2962) and Chames et al. (Nucleic Acids Res., 2005, 33, e178). For example, the cleavage activity of the variant of the invention may be measured by a direct repeat recombination assay, in yeast or mammalian cells, using a reporter vector. The reporter vector comprises two truncated, non-functional copies of a reporter gene (direct repeats) and the genomic DNA target sequence within the intervening sequence, cloned in a yeast or a mammalian expression vector. Expression of the variant results in a functional endonuclease which is able to cleave the genomic DNA target sequence. This cleavage induces homologous recombination between the direct repeats, resulting in a functional reporter gene, whose expression can be monitored by appropriate assay.

Definitions

Amino acid residues in a polypeptide sequence are designated herein according to the one-letter code, in which, for example, Q means Gln or Glutamine residue, R means Arg or Arginine residue and D means Asp or Aspartic acid residue.

Nucleotides are designated as follows: one-letter code is used for designating the base of a nucleoside: a is adenine, t is thymine, c is cytosine, and g is guanine. For the degenerated nucleotides, r represents g or a (purine nucleotides), k represents g or t, s represents g or c, w represents a or t, m represents a or c, y represents t or c (pyrimidine nucleotides), d represents g, a or t, v represents g, a or c, b represents g, t or c, h represents a, t or c, and n represents g, a, t or c.

by “meganuclease”, is intended an endonuclease having a double-stranded DNA target sequence of 14 to 40 pb. Said meganuclease is either a dimeric enzyme, wherein each domain is on a monomer or a monomeric enzyme comprising the two domains on a single polypeptide.

by “meganuclease domain” is intended the region which interacts with one half of the DNA target of a meganuclease and is able to associate with the other domain of the same meganuclease which interacts with the other half of the DNA target to form a functional meganuclease able to cleave said DNA target.

by “meganuclease variant” or “variant” is intented a meganuclease obtained by replacement of at least one residue in the amino acid sequence of the wild-type meganuclease (natural meganuclease) with a different amino acid.

by “functional variant” is intended a variant which is able to cleave a DNA target sequence, preferably said target is a new target which is not cleaved by the parent meganuclease. For example, such variants have amino acid variation at positions contacting the DNA target sequence or interacting directly or indirectly with said DNA target.

by “meganuclease variant with novel specificity” is intended a variant having a pattern of cleaved targets different from that of the parent homing endonuclease. The terms “novel specificity”, “modified specificity”, “novel cleavage specificity”, “novel substrate specificity” which are equivalent and used indifferently, refer to the specificity of the variant towards the nucleotides of the DNA target sequence.

by “I-CreI” is intended the wild-type I-CreI having the sequence SWISSPROT P05725 (SEQ ID NO: 143) or pdb accession code 1g9y (SEQ ID NO: 144).

by “LAGLIDADG core domain” or “core domain” is intended the “LAGLIDADG Homing Endonuclease Core Domain” which is the characteristic α₁β₁β₂α₂β₃β₄α₃ fold of the homing endonucleases of the LAGLIDADG family, corresponding to a sequence of about one hundred amino acid residues. Said domain comprises four beta-strands (β₁, β₂, β₃, β₄) folded in an antiparallel beta-sheet which interacts with one half of the DNA target. This domain is able to associate with another LAGLIDADG Homing Endonuclease Core Domain which interacts with the other half of the DNA target to form a functional endonuclease able to cleave said DNA target. For example, in the case of the dimeric homing endonuclease I-CreI (163 amino acids), the LAGLIDADG Homing Endonuclease Core Domain corresponds to the residues 6 to 94.

by “single-chain meganuclease” “single-chain chimeric meganuclease”, “single-chain chimeric endonuclease”, “single-chain meganuclease derivative”, “single-chain chimeric meganuclease derivative” or “single-chain derivative” is intended a meganuclease comprising two LAGLIDADG Homing Endonuclease domains or core domains linked by a peptidic spacer. The single-chain meganuclease is able to cleave a chimeric DNA target sequence comprising one different half of each parent meganuclease target sequence.

by “subdomain” is intended the region of a LAGLIDADG Homing

Endonuclease Core Domain which interacts with a distinct part of a homing endonuclease DNA target half-site. Two different subdomains behave independently and the mutation in one subdomain does not alter the binding and cleavage properties of the other subdomain. Therefore, two subdomains bind distinct part of a homing endonuclease DNA target half-site.

by “beta-hairpin” is intended two consecutive beta-strands of the antiparallel beta-sheet of a LAGLIDADG homing endonuclease core domain (β₁β₂ or, β₃β₄) which are connected by a loop or a turn,

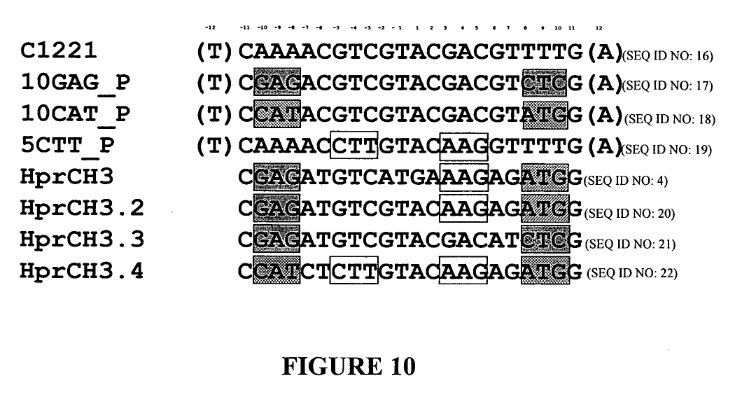

by “I-CreI site” is intended a 22 to 24 by double-stranded DNA sequence which is cleaved by I-CreI. I-CreI sites include the wild-type (natural) non-palindromic I-CreI homing site and the derived palindromic sequences such as the sequence 5′t₋₁₂c₋₁a₋₁₀a₋₉a₋₈a₋₇c₋₆g₋₅t₋₄c₋₃g₋₂t₋₁a₊₁c₊₂g₊₃a₊₄c₊₅g₊₆t₊₇t₊₈t₊₉t₊₁₀g₊₁₁a₊₁₂ also called C1221 (SEQ ID NO:16; FIG. 10).

by “DNA target”, “DNA target sequence”, “target sequence” , “target-site”, “target” , “site”; “site of interest”; “recognition site”, “recognition sequence”, “homing recognition site”, “homing site”, “cleavage site” is intended a 20 to 24 by double-stranded palindromic, partially palindromic (pseudo-palindromic) or non-palindromic polynucleotide sequence that is recognized and cleaved by a LAGLIDADG homing endonuclease such as I-CreI, or a variant, or a single-chain chimeric meganuclease derived from I-CreI. These terms refer to a distinct DNA location, preferably a genomic location, at which a double stranded break (cleavage) is to be induced by the endonuclease. The DNA target is defined by the 5′ to 3′ sequence of one strand of the double-stranded polynucleotide, as indicate above for C1221. Cleavage of the DNA target occurs at the nucleotides in positions +2 and −2, respectively for the sense and the antisense strand. Unless otherwise indicated, the position at which cleavage of the DNA target by an I-Cre I meganuclease variant occurs, corresponds to the cleavage site on the sense strand of the DNA target.

by “DNA target half-site”, “half cleavage site” or half-site” is intended the portion of the DNA target which is bound by each LAGLIDADG homing endonuclease core domain.

by “chimeric DNA target”or “hybrid DNA target” is intended the fusion of a different half of two parent meganuclease target sequences. In addition at least one half of said target may comprise the combination of nucleotides which are bound by at least two separate subdomains(combined DNA target).

by “DNA target sequence from the HPRT gene” is intended a 20 to 24 by sequence of a HPRT gene which is recognized and cleaved by a meganuclease variant.

by “HPRT gene” is intended the HPRT gene of a vertebrate.

by “vector” is intended a nucleic acid molecule capable of transporting another nucleic acid to which it has been linked.

by “homologous” is intended a sequence with enough identity to another one to lead to a homologous recombination between sequences, more particularly having at least 95% identity, preferably 97% identity and more preferably 99%.

“identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default settings.

“individual” includes mammals, as well as other vertebrates (e.g., birds, fish and reptiles). The terms “mammal” and “mammalian”, as used herein, refer to any vertebrate animal, including monotremes, marsupials and placental, that suckle their young and either give birth to living young (eutharian or placental mammals) or are egg-laying (metatharian or nonplacental mammals). Examples of mammalian species include humans and other primates (e.g., monkeys, chimpanzees), rodents (e.g., rats, mice, guinea pigs) and others such as for example: cows, pigs and horses.

by mutation is intended the substitution, deletion, insertion of one or more nucleotides/amino acids in a polynucleotide (cDNA, gene) or a polypeptide sequence. Said mutation can affect the coding sequence of a gene or its regulatory sequence. It may also affect the structure of the genomic sequence or the structure/stability of the encoded mRNA.

“genetic disease” refers to any disease, partially or completely, directly or indirectly, due to an abnormality in one or several genes. Said abnormality can be a mutation. Said genetic disease can be recessive or dominant.

In a preferred embodiment of the use according to the present invention, said substitution(s) in the subdomain situated from positions 44 to 77 of I-CreI are in positions 44, 68, 70, 75 and/or 77.

In another preferred embodiment of the use according to the present invention, said substitution(s) in the subdomain situated from positions 26 to 40 of I-CreI are in positions 26, 28, 30, 32, 33, 38 and/or 40.

In another preferred embodiment of the use according to the present invention, said I-CreI variant or single-chain derivative comprises the substitution of other amino acid residues contacting the DNA target sequence or interacting with the DNA backbone or with the nucleotide bases, directly or via a water molecule; these I-CreI interacting residues are well-known in the art.

In another preferred embodiment of the use according to the present invention, said I-CreI variant or single-chain derivative comprises one or more additional substitutions that improve the binding and/or cleavage activity of the variant towards the DNA target of the HPRT gene as defined above; these substitutions are situated on the entire I-CreI sequence or only in the C-terminal half of I-CreI (positions 80 to 163)

Preferably, said additional substitutions are at a position of I-CreI selected from the group consisting of positions: 2, 9, 19, 42, 43, 54, 66, 69, 72, 81, 82, 86, 90, 92, 96, 100, 103, 104, 105, 107, 108, 109, 110, 113, 120, 125, 129, 130, 131, 132, 135, 136, 137, 140, 143, 151, 154, 155, 157, 158, 159, 161 and 162.

More preferably said substitution is the G19S or G19A mutation which increase the cleavage activity of the I-CreI variant/single-chain derivative. Still more preferably, said mutation is the G19S mutation which further impairs the formation of a functional homodimer. The G19S mutation is advantageously introduced in one of the two monomers of an heterodimeric I-CreI variant, so as to obtain a meganuclease having enhanced cleavage activity and enhanced cleavage specificity.

In another preferred embodiment of the use according to the present invention, said substitutions are replacement of the initial amino acids with amino acids selected from the group consisting of: A, D, E, G, H, K, N, P, Q, R, S, T, Y, C, V, L, W, M and I.

For example:

the lysine (K) in position 28 may be mutated in: R,

the asparagine (N) in position 30 may be mutated in: S, C, R, Y, Q, D and T,

the serine (S) in position 32 may be mutated in: D, T, R, G and W,

the tyrosine (Y) in position 33 may be mutated in: H, T, G, R, C, Q, D and S,

the glutamine (Q) in position 38 may be mutated in: W, S, T, G, E, A, Y, C, D and H

the serine (S) in position 40 may be mutated in: Q, A, T and R,

the glutamine (Q) in position 44 may be mutated in : N, T, R, K, D, Y and A,

the arginine (R) in position 68 may be mutated in: K, Q, E, A, Y, N, H and T,

the arginine (R) in position 70 may be mutated in: S, H, N and K,

the aspartic acid (D) in position 75 may be mutated in: R, S, N, Y, E, H and Q, and

the isoleucine (I) in position 77 may be mutated in: T, W, Y, K, N, R, H, D, F, E, Q and L.

In addition, the I-CreI variants as defined in the present invention may include one or more residues inserted at the NH₂ terminus and/or COOH terminus of the I-CreI sequence. For example, a tag (epitope or polyhistidine sequence) is introduced at the NH₂ terminus and/or COOH terminus; said tag is useful for the detection and/or the purification of said variant.

The I-CreI variant as defined in the invention may be an homodimer or an heterodimer resulting from the association of a first monomer having at least one mutation in positions 26 to 40 or 44 to 77 of I-CreI and a second monomer which is I-CreI or an I-CreI variant.

In another preferred embodiment of the use according to the present invention, said I-CreI variant is an heterodimer, resulting from the association of a first and a second monomer having different mutations in positions 26 to 40 and/or 44 to 77 of I-CreI.

In a more preferred embodiment, at least one monomer has at least two substitutions, one in each of the two functional subdomains situated from positions 26 to 40 and 44 to 77 of I-CreI.

More preferably, said heterodimer consist of a first and a second monomer selected from the following pairs of sequences: SEQ ID NO: 83 and 97, SEQ ID NO: 84 and 98, SEQ ID NO: 85 and 99, SEQ ID NO: 32 and 52, SEQ ID NO: 32 and 53, SEQ ID NO: 32 and 54, SEQ ID NO: 32 and 55, SEQ ID NO: 32 and 56, SEQ ID NO: 32 and 57, SEQ ID NO: 32 and 58, SEQ ID NO: 32 and 60, SEQ ID NO: 32 and 65, SEQ ID NO: 32 and 66, SEQ ID NO: 32 and 67, SEQ ID NO: 32 and 68, SEQ ID NO: 32 and 69, SEQ ID NO: 32 and 70, SEQ ID NO: 32 and 71, SEQ ID NO: 32 and 72, SEQ ID NO: 32 and 73, SEQ ID NO: 32 and 74, SEQ ID NO: 75 and 56, SEQ ID NO: 76 and 56, SEQ ID NO: 77 and 56, SEQ ID NO: 78 and 56, SEQ ID NO: 79 and 56, SEQ ID NO: 80 and 56, SEQ ID NO: 81 and 56, SEQ ID NO: 82 and 56, SEQ ID NO: 86 and 96, SEQ ID NO: 87 and 100, SEQ ID NO: 88 and 101, SEQ ID NO: 89 and 102, SEQ ID NO: 90 and 103, SEQ ID NO: 91 and 104, SEQ ID NO: 92 and 105, SEQ ID NO: 93 and 106, SEQ ID NO: 94 and 107, SEQ ID NO: 95 and 108, SEQ ID NO: 147 and 148.

The single-chain derivative of the I-CreI variant as defined in the present invention is a fusion protein comprising two monomers or two core domains of a LAGLIDADG meganuclease or a combination of both, wherein at least one monomer or core domain has the sequence of an I-CreI variant having at least one substitution in positions 26 to 40 and/or 44 to 77 of I-CreI, as defined above.

The DNA target sequences which are cleaved by the I-CreI variant or single-chain derivative are situated in the HPRT ORF and these sequences cover all the HPRT ORF (Table I and FIG. 2).

TABLE I
Targets position and identity in mammals

| | | | Position | | | | | |
|---|---|---|---|---|---|---|---|---|
| | | | in the | Position | Position | | | |
| | | | Chinese | in the | in the | | | |
| | | | Hamster | mouse | human | Identitity | | |
| | Position | | HPRT | HPRT | HPRT | with the | Identitity | Identitity |
| SEQ | in the | Position | mRNA | mRNA | mRNA | Chinese | with the | with the |
| ID | HPRT | in the | (SEQ ID | (SEQ ID | (SEQ ID | Hamster | mouse | human |
| NO: | gene | Exon¹ | NO: 15) | NO: 145) | NO: 146) | sequence | sequence | sequence |
| 1 | Exon 1 | −19 to +2 | 72-93 | 69-90 | 67-98 | 22/22 | 19/22 | 17/22 |
| 2 | Exon 2 | 42-63 | 159-180 | 156-177 | 154-175 | 22/22 | 19/22 | 18/22 |
| 3 | Exon 2 | 81-102 | 198-219 | 195-216 | 193-214 | 22/22 | 21/22 | 20/22 |
| 4 | Exon 3 | 17-38 | 244-262 | 241-259 | 239-257 | 22/22 | 21/22 | 20/22 |
| 5 | Exon 3 | 57-78 | 281-302 | 278-299 | 276-297 | 22/22 | 21/22 | 21/22 |
| 6 | Exon 3 | 89-110 | 313-334 | 310-331 | 308-329 | 22/22 | 22/22 | 22/22 |
| 7 | Exon 3 | 94-115 | 318-339 | 315-336 | 313-334 | 22/22 | 22/22 | 21/22 |
| 8 | Exon 3 | 138-159 | 372-393 | 369-390 | 367-388 | 22/22 | 21/22² | 21/22² |
| 9 | Exon 6 | 9-30 | 501-522 | 498-547 | 496-545 | 22/22 | 22/22 | 20/22² |
| 10 | Exon 6 | 37-58 | 529-550 | 526-547 | 524-545 | 22/22 | 21/22² | 18/22 |
| 11 | Exon 6 | 38-59 | 530-551 | 527-548 | 525-546 | 22/22 | 21/22² | 18/22 |
| 12 | Exon 8 | 4-25 | 626-647 | 623-644 | 621-642 | 22/22 | 22/22 | 22/22 |
| 13 | Exon 9 | 9-30 | 708-729 | 705-726 | 703-724 | 22/22 | 21/22 | 21/22 |
| 14 | Exon 9 | 46-67 | 745-766 | 742-763 | 740-761 | 22/22 | 19/22³ | 21/22² |

¹The position is relative to the start of the corresponding Exon except for the target SEQ ID NO: 1 whose position is indicated relatively to the ATG initiation codon.
²100% identity at positions ± 3 to 5 and 8 to 10 with no gap
³100% identity at positions ± 3 to 5 and 8 to 10 with gap in the middle

The DNA target sequences are present in the HPRT gene of at least one mammal (human or animal).

For example, the target sequences SEQ ID NO: 6 and 12 are present at least in the human, mouse and Chinese Hamster (Criteculus sp.) HPRT genes.

The target sequences SEQ ID NO: 7 and 9 are present at least in both the mouse and Chinese Hamster HPRT genes.

The target sequences SEQ ID NO: 1 to 5, 8, 10, 11, 13 and 14 are present at least in the Chinese Hamster HPRT gene.

In addition, target sequences having sequence identity with the nucleotides in position ±3 to 5 and ±8 to 10 of the sequences SEQ ID NO: 8 and 14 are present at least in the human and mouse HPRT genes. Target sequences having sequence identity with the nucleotides in position ±3 to 5 and ±8 to 10 of the sequences SEQ ID NO: 10 and 11 are present at least in the mouse HPRT gene (sequence identity is not found with the human HPRT gene). A target sequence having sequence identity with the nucleotides in position ±3 to 5 and ±8 to 10 of the sequence SEQ ID NO: 9 is present at least in the human HPRT gene.

Therefore, the I-CreI variants which cleave one of the DNA target sequences SEQ ID NO: 6 and 12 are able to induce a site-specific modification at least in the human, mouse and Chinese Hamster HPRT gene. In addition, the I-CreI variants which cleave the DNA target sequences SEQ ID NO: 9 are able to induce a site-specific modification both in the Chinese Hamster and mouse HPRT gene, and for some of them, also in the human HPRT gene. The I-CreI variants which cleave the DNA target sequences SEQ ID NO: 8 are able to induce a site-specific modification in the Chinese Hamster and for some of them, also in the human and/or mouse HPRT gene; the position of the modification in the HPRT gene corresponds to the position of the genomic DNA cleavage site (position +2 on the sense strand of the genomic DNA target (i.e. positions: 101 (Exon 3), 16 (Exon 8), 21 (Exon 6), 150 (Exon 3), respectively for the sequences SEQ ID NO: 6, 12, 9 and 8).

The I-CreI variants which cleave the DNA target sequence SEQ ID NO: 7 are able to induce a site-specific modification at least in the mouse and Chinese Hamster HPRT gene (but not at the corresponding position in the human HPRT gene). In addition, the I-CreI variants which cleave the DNA target sequences SEQ ID NO: 10 and 11 are able to induce a site-specific modification in the Chinese Hamster HPRT gene and for some of them, also in the mouse HPRT gene (but not at the corresponding position in the human HPRT gene); the position of the modification in the HPRT gene corresponds to positions 106 (Exon 3), 51(Exon 6) and 52 (Exon 6), respectively.

The I-CreI variants which cleave the DNA target sequence SEQ ID NO: 14 are able to induce a site-specific modification in the Chinese Hamster HPRT gene and for some of them, also in the human HPRT gene (but not at the corresponding position in the mouse HPRT gene); the position of the modification in the HPRT gene corresponds to position 68 (Exon 9).

The I-CreI variants which cleave one of the DNA target sequences SEQ ID NO: 1 to 5 and 13 are able to induce a site-specific modification at least in the Chinese Hamster HPRT gene (but not at the corresponding position in the human or mouse HPRT gene); the position of the modification in the HPRT gene corresponds to positions -7 from the ATG (Exon 1), 54 (Exon 2), 93(Exon 2), 29 (Exon 3), 69(Exon 3), 93(Exon 9) and 21(Exon 9), respectively.

Examples of heterodimeric variants which cleave each DNA target are presented in Table II and FIG. 19.

TABLE II
Sequence of heterodimeric I-CreI variants cleaving having
a DNA target from the HPRT gene

| | Second I-CreI monomer | |
|---|---|---|
| | (SEQ ID NO: 97 to 99, 52 to 58, 60, 65 to 74, | |
| | 56, 148, 96 and 100 to 108) | Target |
| First I-CreI monomer | | |
| (SEQ ID NO: 83, 84, 85, 32, 75 to 82, 147 | | |
| 86 to 95) | | |
| 28K30Q32D33Y38Q40S44N68K70S75R77T | 28K30N32S33G38C40S44Q68R70R75N77I | 1 |
| 28K30D32S33R38Q40S44N68Q70S75S77V | 28K30N32S33T38G40S44T68R70S75Y77T | 2 |
| 28K30T32S33G38Q40S44Q68R70S75R77Y | 28K30N32S33T38Q40R44N68E70S75R77K | 3 |
| 28K30N32S33H38Q40S44Q68R70R75D77I | 28K30N32T33H38Q40S44R68A70S75N77N | 4 |
| | 28K30S32S33Q38Q40S44R68Y70S75D77N | |
| | 28K30N32T33H38Q40S44R68Y70S75D77N | |
| | 28K30N32T33H38Q40S44R68Y70S75D77R | |
| | 28K30N32S33T38Y40S44K68Y70S75E77V | |
| | 28K30N32R33D38Q40S44K68Y70S75D77R | |
| | 28K30N32S33S38D40S44K68Y70S75D77R | |
| | 28K30N32S33Y38Q40S44R68Y70S75N77I | |
| | 28R30N32S33S38Y40Q44R68A70S75N77N | |
| | 28R30N32S33S38Y40Q44R68A70S75H77Y | |
| | 28R30N32T33S38Y40Q44R68Y70S75N77N | |
| | 140M | |
| | 28K30N32T33H38H40S44Q68Y70S75D77R | |
| | 28K30N32T33H38Q40S44K68Y70S75D77R | |
| | 28K30N32T33H38Q40S44Q68N70S75H77R | |
| | 28K30N32T33H38Q40S44Q68R70S75H77R | |
| | 28K30N32T33H38Q40S44Q68H70S75H77H | |
| | 28K30N32T33H38Q40S44Q68H70S75H77H9 | |
| | 2R | |
| | 28K30N32T33H38Q40S44K68Y70S75D77R9 | |
| | 2R96R107R132V140A143A | |
| 28K30N32S33H38Q40S44Q66C68R70R75D77I | 28K30N32S33T38Y40S44K68Y70S75E77V | |
| 137V155R162P | | |
| 9L28K30N32S33H38Q40S44Q68R70R75D77I | | |
| 100I108V154G155P161P | | |
| 2Y28K30N32S33H38Q40S44Q68R70R75D77I | | |
| 109V125A | | |
| 28K30N32S33H38Q40S44Q68R70R75D77I | | |
| 113S136S | | |
| 2I28K30N32S33H38Q40S44Q68R70R75D77I | | |
| 81V86I110G131R135Q151A157V | | |
| 28K30N32S33H38Q40S44Q68R70R71R75D77I | | |
| 103I129A130G | | |
| 28K30N32S33H38Q40S44Q68R69V70R75D77I | | |
| 82R90R120V139R158M | | |
| 28K30N32S33H38Q40S44Q54L68R70R75D77I | | |
| 86D100R104M105A136S159R | | |
| 28K30N32S33H38Q4042A43LS44Q68R70R75D | 28K30N32T33H38Q40S44R68Y70S72T75N | |
| 77I | 77N | |
| First I-CreI monomer | | |
| (SEQ ID NO: 83, 84, 85, 32, 75 to 82, | | |
| 86 to 95) | | |
| 28K30N32T33Y38W40S44N68R70S75Y77I | 28K30R32S33Y38E40S44Q68H70H75N77I | 5 |
| 28K30N32S33T38Q40A44N68K70S75R77T | 28K30N32S33H38T40S44N68R70N75N77I | 6 |
| 28K30N32S33H38S40S44R68R70S75N77D | 28K30N32S33G38Q40Q44Q68A70K75N77I | 7 |
| 28K30N32S33G38T40S44N68K70S75H77F | 28K30C32S33T38Q40S44Q68Y70S75R77Q | 8 |
| 28K30K32S33R38Q40S44D68Y70S75S77R | 28K30N32S33T38Q40Q44N68K70S75H77F | 9 |
| 28K30N32S33T38Q40T44R68Y70S75E77Y | 28K30N32S33R38T40S44R68T70S75N77N | 10 |
| 28K30N32T33T38Q40S44Y68R70S75Y77V | 28K30N32G33T38Q40S44N68Y70S75R77Y | 11 |
| 28K30Y32T33C38Q40S44Q68R70S75D77K | 28K30N32W33T38Q40S44Q68R70R75N77I | 12 |
| 28K30N32S33H38G40S44N68R70S75Y77N | 28K30N32S33T38A40S44Q68R70S75N77L | 13 |
| 28K30N32S33Y38Q40S44A68R70S75Q77E | 28K30N32S33C38Y40S44R68Y70S75D77I | 14 |

The sequence of each variant is defined by its amino acid residues at the indicated positions. For example, the first heterodimeric variant of Table II consists of a first monomer having K, Q, D, Y, Q, S, N, K, S, R and T in positions 28, 30, 32, 33, 38, 40, 44, 68, 70, 75 and 77, respectively and a second monomer having K, N, S, G, C, S, Q, R, R, N, and I in positions 28, 30, 32, 33, 38, 40, 44, 68, 70, 75 and 77, respectively. The positions are indicated by reference to I-CreI sequence SWISSPROT P05725 or pdb accession code 1g9y; I-CreI has K, N, S, Y, Q, S, Q, R, R, D and I in positions 28, 30, 32, 33, 38, 40, 44, 68, 70, 75 and 77, respectively. The positions which are not indicated are not mutated and thus correspond to the wild-type I-CreI sequence.

In another preferred embodiment of the use according to the present invention, said I-CreI variant or single-chain derivative are combined with a targeting DNA construct comprising a sequence to be introduced flanked by sequences sharing homologies with the regions of the HPRT gene surrounding the genomic DNA cleavage site of said I-CreI variant or single-chain derivative, as defined above.

Preferably, homologous sequences of at least 50 bp, preferably more than 100 by and more preferably more than 200 by are used. Indeed, shared DNA homologies are located in regions flanking upstream and downstream the site of the break and the DNA sequence to be introduced should be located between the two arms. The sequence to be introduced comprises an exogenous gene of interest or a sequence to inactivate or delete the HPRT gene or part thereof.

Such chromosomal DNA alterations can be used for making HPRT knock-out and knock-in animals wherein the HPRT gene is inactivated (knock-out) and eventually replaced with an exogenous gene of interest (knock-in).

Accordingly, such chromosomal DNA alterations are used also for making genetically modified vertebrate (mammalian including human) cell lines wherein the endogeneous HPRT gene is inactivated and a transgene is eventually inserted at the HPRT locus.

In addition, following inactivation of the endogenous HPRT gene, HPRT may be used as a positive selection marker (selection for HPRT marker expression with HAT) in further gene targeting procedures at any locus of the chromosomes of the HPRT deficient cell/animal.

The subject-matter of the present invention is also a method for making an HPRT knock-in or knock-out animal, comprising at least the step of:

(a) introducing into a pluripotent precursor cell or an embryo of an animal, an I-CreI variant or single-chain derivative, as defined above, so as to into induce a double stranded cleavage at a site of interest of the HPRT gene comprising a DNA recognition and cleavage site of said I-CreI variant or single-chain derivative, simultaneously or consecutively,

(b) introducing into the animal precursor cell or embryo of step (a) a targeting DNA, wherein said targeting DNA comprises (1) DNA sharing homologies to the region surrounding the cleavage site and (2) DNA which repairs the site of interest upon recombination between the targeting DNA and the chromosomal DNA, so as to generate a genomically modified animal precursor cell or embryo having repaired the site of interest by homologous recombination,

(c) developing the genomically modified animal precursor cell or embryo of step (b) into a chimeric animal, and

(d) deriving a transgenic animal from the chimeric animal of step (c).

Preferably, step (c) comprises the introduction of the genomically modified precursor cell generated in step (b) into blastocysts so as to generate chimeric animals.

The subject-matter of the present invention is also a method for making an HPRT knock-in or knock-out cell, comprising at least the step of:

(a) introducing into a cell, an I-CreI variant or single-chain derivative, as defined above, so as to into induce a double stranded cleavage at a site of interest of the HPRT gene comprising a DNA recognition and cleavage site for said I-CreI variant or single-chain derivative, simultaneously or consecutively,

(b) introducing into the cell of step (a), a targeting DNA, wherein said targeting DNA comprises (1) DNA sharing homologies to the region surrounding the cleavage site and (2) DNA which repairs the site of interest upon recombination between the targeting DNA and the chromosomal DNA, so as to generate a recombinant cell having repaired the site of interest by homologous recombination,

(c) isolating the recombinant cell of step (b), by any appropriate mean.

The targeting DNA is introduced into the cell under conditions appropriate for introduction of the targeting DNA into the site of interest.

In a preferred embodiment, said targeting DNA construct is inserted in a vector.

Alternatively, the HPRT gene may be inactivated by repair of the double-strands break by non-homologous end joining (FIG. 3B).

The subject-matter of the present invention is also a method for making an HPRT knock-out animal, comprising at least the step of:

(a) introducing into a pluripotent precursor cell or an embryo of an animal, an I-CreI variant or single-chain derivative, as defined above, so as to induce a double stranded cleavage at a site of interest of the HPRT gene comprising a DNA recognition and cleavage site of said I-CreI variant or single-chain derivative, and thereby generate genomically modified precursor cell or embryo having repaired the double-strands break by non-homologous end joining,

(b) developing the genomically modified animal precursor cell or embryo of step (a) into a chimeric animal, and

(c) deriving a transgenic animal from a chimeric animal of step (b).

Preferably, step (b) comprises the introduction of the genomically modified precursor cell obtained in step (a), into blastocysts, so as to generate chimeric animals.

The subject-matter of the present invention is also a method for making an HPRT-deficient cell, comprising at least the step of:

(a) introducing into a cell, an I-CreI variant or single-chain derivative, as defined above, so as to induce a double stranded cleavage at a site of interest of the HPRT gene comprising a DNA recognition and cleavage site of said I-CreI variant or single-chain derivative, and thereby generate genomically modified HPRT deficient cell having repaired the double-strands break, by non-homologous end joining, and

(b) isolating the genomically modified HPRT deficient cell of step(a), by any appropriate mean.

The cell which is modified may be any cell of interest. For making transgenic/knock-out animals, the cells are pluripotent precursor cells such as embryo-derived stem (ES) cells, which are well-known in the art. Said I-CreI variant/single-chain derivative can be provided directly to the cell or through an expression vector comprising the polynucleotide sequence encoding said meganuclease and suitable for its expression in the used cell.

The animal is preferably a mammal, more preferably a laboratory rodent (mice, rat, guinea-pig), or a cow, pig, horse or goat.

In addition, the loss of the endogenous HPRT gene in the modified cells may be selected by using the purine analogue 6-thioguanine (6-TG).

In another preferred embodiment of the use according to the present invention, said I-CreI variant or single-chain derivative are encoded by a polynucleotide fragment. Said polynucleotide may encode one monomer of an homodimeric or heterodimeric variant, or two domains/monomers of a single-chain chimeric endonuclease.

In a more preferred embodiment, said polynucleotide fragment is inserted in a vector which is suitable for its expression in the used cells. Said vector comprises advantageously a targeting DNA construct as defined above. Preferably, said vector comprises two different polynucleotide fragments, each encoding one of the monomers of an heterodimeric I-Cre I variant, as defined above.

A vector which can be used in the present invention includes, but is not limited to, a viral vector, a plasmid, a RNA vector or a linear or circular DNA or RNA molecule which may consists of a chromosomal, non chromosomal, semi-synthetic or synthetic nucleic acids. Preferred vectors are those capable of autonomous replication (episomal vector) and/or expression of nucleic acids to which they are linked (expression vectors). Large numbers of suitable vectors are known to those of skill in the art and commercially available.

Viral vectors include retrovirus, adenovirus, parvovirus (e. g. adeno-associated viruses), coronavirus, negative strand RNA viruses such as orthomyxovirus (e. g., influenza virus), rhabdovirus (e. g., rabies and vesicular stomatitis virus), para-myxovirus (e. g. measles and Sendai), positive strand RNA viruses such as piconavirus and alphavirus, and double-stranded DNA viruses including adenovirus, herpesvirus (e. g., Herpes Simplex virus types 1 and 2, Epstein-Barr virus, cytomegalovirus), and poxvirus (e. g., vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosissarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996).

Preferred vectors include lentiviral vectors, and particularly self inactivacting lentiviral vectors.

Vectors can comprise selectable markers, for example: neomycin phosphotransferase, histidinol dehydrogenase, dihydrofolate reductase, hygromycin phosphotransferase, herpes simplex virus thymidine kinase, adenosine deaminase, glutamine synthetase, and hypoxanthine-guanine phosphoribosyl transferase for eukaryotic cell culture; TRP1 for S. cerevisiae; tetracycline, rifampicin or ampicillin resistance in E. coli.

Preferably said vectors are expression vectors, wherein the sequence(s) encoding the variant/single-chain derivative of the invention is placed under control of appropriate transcriptional and translational control elements to permit production or synthesis of said variant. Therefore, said polynucleotide is comprised in an expression cassette. More particularly, the vector comprises a replication origin, a promoter operatively linked to said encoding polynucleotide, a ribosome-binding site, an RNA-splicing site (when genomic DNA is used), a polyadenylation site and a transcription termination site. It also can comprise an enhancer. Selection of the promoter will depend upon the cell in which the polypeptide is expressed. Preferably, when said variant is an heterodimer, the two polynucleotides encoding each of the monomers are included in one vector which is able to drive the expression of both polynucleotides, simultaneously. Suitable promoters include tissue specific and/or inducible promoters. Examples of inducible promoters are: eukaryotic metallothionine promoter which is induced by increased levels of heavy metals, prokaryotic lacZ promoter which is induced in response to isopropyl-β-D-thiogalacto-pyranoside (IPTG) and eukaryotic heat shock promoter which is induced by increased temperature. Examples of tissue specific promoters are skeletal muscle creatine kinase, prostate-specific antigen (PSA), α-antitrypsin protease, human surfactant (SP) A and B proteins, β-casein and acidic whey protein genes.

The targeting DNA is introduced into the cell under conditions appropriate for introduction of the targeting DNA into the site of interest.

For making knock-in animals/cells the DNA which repairs the site of interest comprises the sequence of an exogenous gene of interest, and eventually a selection marker, such as the HPRT gene.

For making knock-out animals/cells, the DNA which repairs the site of interest comprises sequences that inactivate the endogeneous gene of interest.

The subject matter of the present invention is also to the use of an I-CreI variant or a single-chain derivative as defined above, for the preparation of a medicament for preventing, improving or curing a genetic disease associated with a mutation in the HPRT gene in an individual in need thereof, said medicament being administrated by any means to said individual.

In this case, the use of the I-CreI variant or a single-chain derivative as defined above, comprises at least the step of (a) inducing in somatic tissue(s) of the individual a double stranded cleavage at a site of interest of the HPRT gene comprising at least one recognition and cleavage site of said variant, and (b) introducing into the individual a targeting DNA, wherein said targeting DNA comprises (1) DNA sharing homologies to the region surrounding the cleavage site and (2) DNA which repairs the site of interest upon recombination between the targeting DNA and the chromosomal DNA. The targeting DNA is introduced into the individual under conditions appropriate for introduction of the targeting DNA into the site of interest.

According to the present invention, said double-stranded cleavage is induced, either in toto by administration of said meganuclease to an individual, or ex vivo by introduction of said meganuclease into somatic cells removed from an individual and returned into the individual after modification.

In a preferred embodiment of said use, the I-CreI variant or single-chain derivative is combined with a targeting DNA construct comprising a sequence which repairs a mutation in the HPRT gene flanked by sequences sharing homologies with the regions of the HPRT gene surrounding the genomic DNA cleavage site of said I-CreI variant or single-chainderivative, as defined above.

For correcting the HPRT gene, cleavage of the gene occurs in the vicinity of the mutation, preferably, within 500 by of the mutation (FIG. 3C). The targeting construct comprises a HPRT gene fragment which has at least 200 by of homologous sequence flanking the genomic DNA cleavage site (minimal repair matrix) for repairing the cleavage, and includes the correct sequence of the HPRT gene for repairing the mutation (FIG. 3C). Consequently, the targeting construct for gene correction comprises or consists of the minimal repair matrix; it is preferably from 200 pb to 6000 pb, more preferably from 1000 pb to 2000 pb.

For restoring a functional gene (FIG. 3D), cleavage of the gene occurs upstream of a mutation. Preferably said mutation is the first known mutation in the sequence of the gene, so that all the downstream mutations of the gene can be corrected simultaneously. The targeting construct comprises the exons downstream of the genomic DNA cleavage site fused in frame (as in the cDNA) and with a polyadenylation site to stop transcription in 3′. The sequence to be introduced (exon knock-in construct) is flanked by introns or exons sequences surrounding the cleavage site, so as to allow the transcription of the engineered gene (exon knock-in gene) into a mRNA able to code for a functional protein (FIG. 3D). For example, the exon knock-in construct is flanked by sequences upstream and downstream

In another preferred embodiment of said use, the I-CreI variant or single-chain derivative is encoded by a vector. Preferably, the vector comprises the targeting DNA construct, as defined above.

In another preferred embodiment of said use, the genetic disease is the Lesch Nyhan Syndrome.

The subject-matter of the present invention is also a composition characterized in that it comprises at least one I-CreI variant or single-chain derivative and/or at least one expression vector encoding said variant/single-chain molecule, as defined above, and a pharmaceutically acceptable excipient.

In a preferred embodiment of said composition, it comprises a targeting DNA construct comprising a sequence which repairs a mutation in the HPRT gene, flanked by sequences sharing homologies with the genomic DNA cleavage site of said variant, as defined above. The sequence which repairs the mutation is either a fragment of the gene with the correct sequence or an exon knock-in construct, as defined above.

Preferably, said targeting DNA construct is either included in a recombinant vector or it is included in an expression vector comprising the polynucleotide(s) encoding the variant/single-chain derivative, as defined in the present invention.

The subject-matter of the present invention is also products containing at least one I-CreI variant/single-chain derivative or one expression vector encoding said meganucleases, and a vector including a targeting construct, as defined above, as a combined preparation for simultaneous, separate or sequential use in the prevention or the treatment of a genetic disease associated with a mutation in the HPRT gene.

The subject-matter of the present invention is also a method for preventing, improving or curing a genetic disease associated with a mutation in the HPRT gene in an individual in need thereof, said method comprising at least the step of administering to said individual a composition as defined above, by any means.

For purposes of therapy, the I-CreI variant/single-chain derivative and a pharmaceutically acceptable excipient are administered in a therapeutically effective amount. Such a combination is said to be administered in a “therapeutically effective amount” if the amount administered is physiologically significant. An agent is physiologically significant if its presence results in a detectable change in the physiology of the recipient. In the present context, an agent is physiologically significant if its presence results in a decrease in the severity of one or more symptoms of the targeted disease and in a genome correction of the lesion or abnormality.

In one embodiment of the uses according to the present invention, the I-CreI variant/single-chain derivative is substantially non-immunogenic, i.e., engender little or no adverse immunological response. A variety of methods for ameliorating or eliminating deleterious immunological reactions of this sort can be used in accordance with the invention. In a preferred embodiment, the I-CreI variant/single-chain derivative is substantially free of N-formyl methionine. Another way to avoid unwanted immunological reactions is to conjugate meganucleases to polyethylene glycol (“PEG”) or polypropylene glycol (“PPG”) (preferably of 500 to 20,000 daltons average molecular weight (MW)). Conjugation with PEG or PPG, as described by Davis et al. (U.S. Pat. No. 4,179,337) for example, can provide non-immunogenic, physiologically active, water soluble endonuclease conjugates with anti-viral activity. Similar methods also using a polyethylene—polypropylene glycol copolymer are described in Saifer et al. (U.S. Pat. No. 5,006,333).

The I-CreI variant or single-chain derivative can be used either as a polypeptide or as a polynucleotide construct/vector encoding said polypeptide. It is introduced into cells, in vitro, ex vivo or in vivo, by any convenient means well-known to those in the art, which are appropriate for the particular cell type, alone or in association with either at least an appropriate vehicle or carrier and/or with the targeting DNA. Once in a cell, the meganuclease and if present, the vector comprising targeting DNA and/or nucleic acid encoding a meganuclease are imported or translocated by the cell from the cytoplasm to the site of action in the nucleus.

The I-CreI variant or single-chain derivative (polypeptide) may be advantageously associated with: liposomes, polyethyleneimine (PEI), and/or membrane translocating peptides (Bonetta, The Scientist, 2002, 16, 38; Ford et al., Gene Ther., 2001, 8, 1-4 ; Wadia and Dowdy, Curr. Opin. Biotechnol., 2002, 13, 52-56); in the latter case, the sequence of the I-Cre variant/single-chain derivative is fused with the sequence of a membrane translocating peptide (fusion protein).

Vectors comprising targeting DNA and/or nucleic acid encoding a meganuclease can be introduced into a cell by a variety of methods (e.g., injection, direct uptake, projectile bombardment, liposomes, electroporation). Meganucleases can be stably or transiently expressed into cells using expression vectors. Techniques of expression in eukaryotic cells are well known to those in the art. (See Current Protocols in Human Genetics: Chapter 12 “Vectors For Gene Therapy” & Chapter 13 “Delivery Systems for Gene Therapy”). Optionally, it may be preferable to incorporate a nuclear localization signal into the recombinant protein to be sure that it is expressed within the nucleus.

The subject-matter of the present invention is also an I-CreI variant/single-chain derivative, a polynucleotide fragment encoding said variant or a single-chain derivative, a vector comprising said polynucleotide fragment and/or a DNA targeting construct, a prokaryotic or eukaryotic host cell which is modified by a polynucleotide or a vector as defined above, preferably an expression vector.

The subject-matter of the present invention is also a non-human transgenic animal or a transgenic plant, wherein all or part of their cells are modified by a polynucleotide or a vector as defined above.

As used herein, a cell refers to a prokaryotic cell, such as a bacterial cell, or an eukaryotic cell, such as an animal, plant or yeast cell.

The I-CreI variant as defined in the present invention is obtainable by a method for engineering I-CreI variants able to cleave a genomic DNA target sequence from a vertebrate gene, comprising at least the steps of:

(a) constructing a first series of I-CreI variants having at least one substitution in a first functional subdomain of the LAGLIDADG core domain situated from positions 26 to 40 of I-CreI,

(b) constructing a second series of I-CreI variants having at least one substitution in a second functional subdomain of the LAGLIDADG core domain situated from positions 44 to 77 of I-CreI,

(c) selecting and/or screening the variants from the first series of step (a) which are able to cleave a mutant I-CreI site wherein (i) the nucleotide triplet in positions −10 to −8 of the I-CreI site has been replaced with a nucleotide triplet selected from the group consisting of cag, att, cct, ttg, gac, atg, ttt, ttc, tgg, gtc, aag, gag and (ii) the nucleotide triplet in positions +8 to +10 has been replaced with the reverse complementary sequence of said nucleotide triplet which is substituted in position −10 to −8 of said I-CreI site (i.e.: ctg, aat, agg, caa, gtc, cat, aaa, gaa, cca, gac, at, and ctc, respectively),

(d) selecting and/or screening the variants from the second series of step (b) which are able to cleave a mutant I-CreI site wherein (i) the nucleotide triplet in positions −5 to −3 of the I-CreI site has been replaced with a nucleotide triplet selected from the group consisting of : gac, taa, tca, gtg, gct, tgt, tgg, ctg, ttg, tag, and gag and (ii) the nucleotide triplet in positions +3 to +5 has been replaced with the reverse complementary sequence of said nucleotide triplet which is substituted in position −5 to −3 of said I-CreI site (i.e.: gtc, tta, tga, cac, agc, aca, cca, cag, caa, cta and ctc, respectively),

(e) selecting and/or screening the variants from the first series of step (a) which are able to cleave a mutant I-CreI site wherein (i) the nucleotide triplet in positions +8 to +10 of the I-CreI site has been replaced a nucleotide triplet selected from the group consisting of: cat, cga, tat, ggg, tac, taa, cag, gca, aca, gaa, tga, atg, and (ii) the nucleotide triplet in positions −10 to −8 has been replaced with the reverse complementary sequence of said nucleotide triplet which is substituted in position +8 to +10 of said I-CreI site (i.e.: atg, tcg, ata, ccc, gta, tta, ctg, tgc, tgt, ttc, tca and cat, respectively),

(f) selecting and/or screening the variants from the second series of step (b) which are able to cleave a mutant I-CreI site wherein (i) the nucleotide triplet in positions +3 to +5 of the I-CreI site has been replaced with the nucleotide triplet selected from the group consisting of: tcc, tat, gtg, gaa, tgg, tac, ttt, aca, agc, gcg, tcc, act, caa and aag and (ii) the nucleotide triplet in positions −5 to −3 has been replaced with the reverse complementary sequence of which is substituted in position +3 to +5 of said I-CreI site (i.e.: gga, ata, cac, ttc, cca, gta, aaa, tgt, gct, cgc, gga, agt, ttg and ctt, respectively),

(gᵢ) selecting and/or screening the variants from steps (c) to (f) which are able to cleave a DNA target of the sequence SEQ ID NO: 1 to 14.

According to a first embodiment of the invention, said I-CreI variant is obtainable by a method comprising at least the steps (a) to (f) as defined above, and the further steps of:

(g2) combining different variants obtained in any of step (c) to (f) with each other or with I-CreI, to form heterodimers, and

(h₂) selecting and/or screening the heterodimers from step (g₂) which are able to cleave said DNA target of the sequence SEQ ID NO: 1 to 14.

According to a second embodiment of the invention, said I-CreI variant is obtainable by a method comprising at least the steps (a) to (f) as defined above, and the further steps of:

(g₃) combining in a single variant, the mutation(s) in positions 26 to 40 and 44 to 77 of two variants from step (c) and step (d), to obtain a novel homodimeric I-CreI variant which cleaves a sequence wherein (i) the nucleotide triplet in positions −10 to −8 is identical to the nucleotide triplet which is present in positions −10 to −8 of said DNA target of the sequence SEQ ID NO: 1 to 14, (ii) the nucleotide triplet in positions +8 to +10 is identical to the reverse complementary sequence of the nucleotide triplet which is present in positions −10 to −8 of said DNA target of the sequence SEQ ID NO: 1 to 14, (iii) the nucleotide triplet in positions −5 to −3 is identical to the nucleotide triplet which is present in positions −5 to −3 of said DNA target of the sequence SEQ ID NO: 1 to 14 (iv) the nucleotide triplet in positions +3 to +5 is identical to the reverse complementary sequence of the nucleotide triplet which is present in positions −5 to −3 of said of said DNA target of the sequence SEQ ID NO: 1 to 14, and/or,

(h₃) combining in a single variant, the mutation(s) in positions 26 to 40 and 44 to 77 of two variants from step (e) and step (f), to obtain a novel homodimeric I-CreI variant which cleaves a sequence wherein (i) the nucleotide triplet in positions +3 to +5 is identical to the nucleotide triplet which is present in positions +3 to +5 of said of said DNA target of the sequence SEQ ID NO: 1 to 14, (ii) the nucleotide triplet in positions −5 to −3 is identical to the reverse complementary sequence of the nucleotide triplet which is present in positions +3 to +5 of said of said DNA target of the sequence SEQ ID NO: 1 to 14, (iii) the nucleotide triplet in positions +8 to +10 of the I-CreI site has been replaced with the nucleotide triplet which is present in positions +8 to +10 of said of said DNA target of the sequence SEQ ID NO: 1 to 14 and (iv) the nucleotide triplet in positions −10 to −8 is identical to the reverse complementary sequence of the nucleotide triplet in positions +8 to +10 of said of said DNA target of the sequence SEQ ID NO: 1 to 14, and

(i₃) selecting and/or screening the variants from steps (g₃) or (h₃) which are able to cleave a DNA target of the sequence SEQ ID NO: 1 to 14.

According to a third embodiment of the invention, said I-CreI variant is obtainable by a method comprising at least the steps (a) to (f), the step (g₃) and/or the step (h₃) as defined above, and the further steps of :

(i₄) combining the variants obtained in step (g₃) with the variants obtained in step (h₃), I-CreI or the variants obtained in step (e) or step (f), to form heterodimers, or

(i′₄) combining the variants obtained in step (h₃) with I-CreI or the variants obtained in step (c) or step (d), to form heterodimers, and

(j4) selecting and/or screening the heterodimers from step (i₄) ₒᵣ (i′₄) which are able to cleave a DNA target of the sequence SEQ ID NO: 1 to 14.

The selection and/or screening in steps (c), (d), (e), (f), (g₁), (h₂), (i₃) and (j₄) may be performed by using a cleavage assay in vitro or in vivo, as described in the International PCT Application WO 2004/067736, Epinat et al. (Nucleic Acids Res., 2003, 31, 2952-2962), Chames et al. (Nucleic Acids Res., 2005, 33, e178), and Arnould et al. (J. Mol. Biol., 2006, 355, 443-458). Preferably, steps (c), (d), (e), (gᵢ), (h₂), (i₃) and/or (j₄) are performed in vivo, under conditions where the double-strand break in the mutated DNA target sequence which is generated by said variant leads to the activation of a positive selection marker or a reporter gene, or the inactivation of a negative selection marker or a reporter gene, by recombination-mediated repair of said DNA double-strand break, as described in the International PCT Application WO 2004/067736, Epinat et al. (Nucleic Acids Res., 2003, 31, 2952-2962), Chames et al. (Nucleic Acids Res., 2005, 33, e178), and Arnould et al. (J. Mol. Biol., 2006, 355, 443-458).

Steps (a) and (b) may comprise the introduction of additional mutations in order to improve the binding and/or cleavage properties of the mutants, particularly at other positions contacting the DNA target sequence or interacting directly or indirectly with said DNA target. These steps may be performed by generating combinatorial libraries as described in the International PCT Application WO 2004/067736 and Arnould et al. (J. Mol. Biol., 2006, 355, 443-458).

The (intermolecular) combination of the variants in step (g₂), (i₄), and (i′₄) is performed by co-expressing, either two different variants from steps (c) and (d), (e) and (f), (g₃) and (h₃), (g₃) and (e), (g₃) and (f), (h₃) and (c), (h₃) and (d), or one variant from any of steps (c) to (f), (g₃) or (h₃) with I-CreI, so as to allow the formation of heterodimers. For example, host cells may be modified by one or two recombinant expression vector(s) encoding said variant(s). The cells are then cultured under conditions allowing the expression of the variant(s), so that heterodimers are formed in the host cells, as described previously in the International PCT Application WO 2006/097854 and Arnould et al. (J. Mol. Biol., 2006, 355, 443-458).

The (intramolecular) combination of mutations in steps (g₃) and (h₃) may be performed by amplifying overlapping fragments comprising each of the two subdomains by well-known overlapping PCR techniques.

In addition, step (g₃) and/or (h₃) may further comprise the introduction of random mutations on the whole variant or in a part of the variant, in particular the C-terminal half of the variant (positions 80 to 163). This may be performed by generating random mutagenesis libraries on a pool of variants, according to standard mutagenesis methods which are well-known in the art and commercially available.

The subject matter of the present invention is also an I-CreI variant having mutations in positions 26 to 40 and/or 44 to 77 of I-CreI that is useful for engineering the variants able to cleave a DNA target from the HPRT gene, according to the present invention. In particular, the invention encompasses the I-CreI variants as defined in step (c) to (f) of the method for engineering I-CreI variants, as defined above, including the variants of the sequence SEQ ID NO: 24 to 47 and 129 to 142. The invention encompasses also the I-CreI variants as defined in step (g₃) and (h₃) of the method for engineering I-CreI variants, as defined above, including the variants of the sequence SEQ ID NO: 52 to 60.

Single-chain chimeric endonucleases able to cleave a DNA target from the gene of interest are derived from the variants according to the invention by methods well-known in the art (Epinat et al., Nucleic Acids Res., 2003, 31, 2952-62; Chevalier et al., Mol. Cell., 2002, 10, 895-905; Steuer et al., Chembiochem., 2004, 5, 206-13; International PCT Applications WO 03/078619 and WO 2004/031346). Any of such methods, may be applied for constructing single-chain chimeric endonucleases derived from the variants as defined in the present invention.

The polynucleotide fragments having the sequence of the targeting DNA construct or the sequence encoding the I-CreI variant or single-chain derivative as defined in the present invention, may be prepared by any method known by the man skilled in the art. For example, they are amplified from a DNA template, by polymerase chain reaction with specific primers. Preferably the codons of the cDNAs encoding the I-CreI variant or single-chain derivative are chosen to favour the expression of said proteins in the desired expression system.

The recombinant vector comprising said polynucleotides may be obtained and introduced in a host cell by the well-known recombinant DNA and genetic engineering techniques.

The I-CreI variant or single-chain derivative as defined in the present the invention are produced by expressing the polypeptide(s) as defined above; preferably said polypeptide(s) are expressed or co-expressed (in the case of the variant only) in a host cell or a transgenic animal/plant modified by one expression vector or two expression vectors (in the case of the variant only), under conditions suitable for the expression or co-expression of the polypeptide(s), and the variant or single-chain derivative is recovered from the host cell culture or from the transgenic animal/plant.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology (Frederick M. AUSUBEL, 2000, Wiley and son Inc, Library of Congress, USA); Molecular Cloning: A Laboratory Manual, Third Edition, (Sambrook et al, 2001, Cold Spring Harbor, New York: Cold Spring Harbor Laboratory Press); Oligonucleotide Synthesis (M. J. Gait ed., 1984); Mullis et al. U.S. Pat. No. 4,683,195; Nucleic Acid Hybridization (B. D. Harries & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods In ENZYMOLOGY (J. Abelson and M. Simon, eds.-in-chief, Academic Press, Inc., New York), specifically, Vols.154 and 155 (Wu et al. eds.) and Vol. 185, “Gene Expression Technology” (D. Goeddel, ed.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); and Manipulating the Mouse Embryo, (Cold Spring Harbor Laboratory Press, Cold Spring Harbor, N.Y., 1986).

In addition to the preceding features, the invention further comprises other features which will emerge from the description which follows, which refers to examples illustrating the I-CreI meganuclease variants and their uses according to the invention, as well as to the appended drawings in which:

FIG. 1 illustrates the modular structure of homing endonucleases and the combinatorial approach for designing custom meganucleases. A. Tridimensional structure of the I-CreI homing endonuclease bound to its DNA target. The catalytic core is surrounded by two αββαββα folds forming a saddle-shaped interaction interface above the DNA major groove. B. Different binding sequences derived from the I-CreI target sequence (top right and bottom left) can be combined to obtain heterodimers or singlechain fusion molecules cleaving non palindromic chimeric targets (bottom right). C. The identification of smaller independent subunits, i. e., a subunit within a single monomer or αββαββα fold (top right and bottom left) would allow for the design of novel chimeric molecules (bottom right), by combination of mutations within the same monomer. Such molecules cleave palindromic chimeric targets (bottom right). D. The combination of the two former steps would allow a larger combinatorial approach, involving four different subdomains. In a first step, couples of novel meganucleases could be combined in new molecules (“half-meganucleases”) cleaving palindromic targets derived from the target one wants to cleave. Then, the combination of such “half-meganuclease” can result in an heterodimeric species cleaving the target of interest. Thus, the identification of a small number of new cleavers for each subdomain would allow for the design of a very large number of novel endonucleases with tailored specificities.

FIG. 2 represents the Hypoxanthine-Guanine Phosphoribosyl

Transferase gene and the corresponding mRNA. The exons are boxed and the size of each exon in the mouse gene (accession number NC_{—}000086) is indicated; differences in size with the human gene (NC_{—}000023) are also indicated. The cleavage sites (SEQ ID NO: 1 to 14) of the I-CreI variants are indicated above the exons. The Criteculus sp. HPRT mRNA (accession number J00060.1; SEQ ID NO: 15) is represented below the gene. The ORF is indicated as a grey box. The HprCH3 target site is indicated with its sequence (SEQ ID NO: 4) and position.

FIG. 3 illustrates four different strategies for the utilization of a meganuclease cleaving the Hypoxanthine-Guanine Phosphoribosyl Transferase (HPRT) gene. A. Gene insertion and/or gene inactivation. Upon cleavage by a meganuclease and recombination with a repair matrix containing a gene of interest (gene insertion) or an inactivation cassette (gene inactivation), flanked by sequences sharing homology with the sequences surrounding the cleavage site, gene insertion or gene inactivation occurs. B. Gene inactivation by non-homologous end-joining. Upon cleavage by a meganuclease, the DNA ends are degraded and rejoined by Non-Homologous-End-Joining (NHEJ), and gene inactivation occurs. C. Gene correction. A mutation occurs within the HPRT gene. Upon cleavage by a meganuclease and recombination with a repair matrix the deleterious mutation is corrected. D. Exonic sequences knock-in. A mutation occurs within the HPRT gene. The mutated mRNA transcript is featured below the gene. In the repair matrix, exons located downstream of the cleavage site are fused in frame (as in a cDNA), with a polyadenylation site to stop transcription in 3′. Introns and exons sequences can be used as homologous regions. Exonic sequences knock-in results into an engineered gene, transcribed into a mRNA able to code for a functional protein.

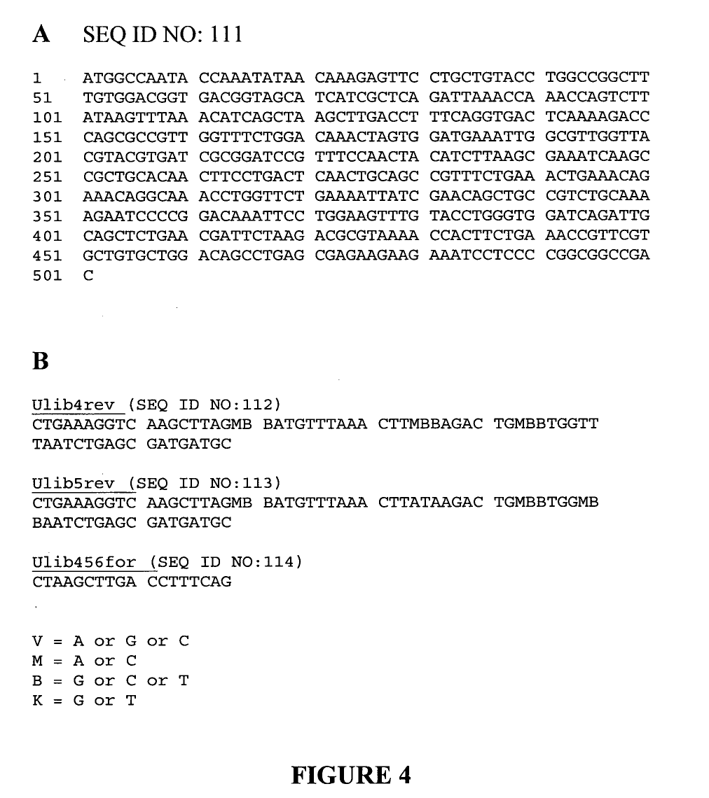

FIG. 4 represents the nucleotide sequence encoding the I-CreI N75 scaffold protein and the sequences of the degenerated primers used for the Ulib4 and Ulib5 libraries construction. A. The scaffolf (SEQ ID NO: 111) is the I-CreI ORF including the D75N codon substitution, the insertion of an alanine (A) codon after the ATG initiation codon and three additional codons (AAD) at the 3′ end. B. Primers (SEQ ID NO: 112, 113, 114),

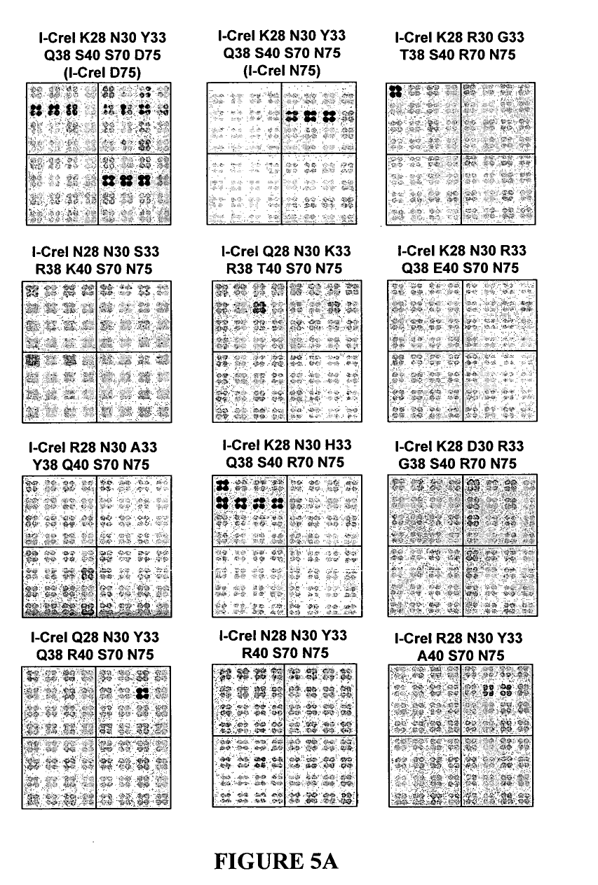

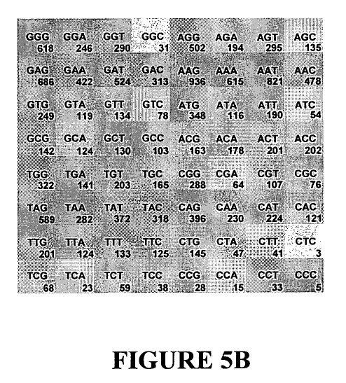

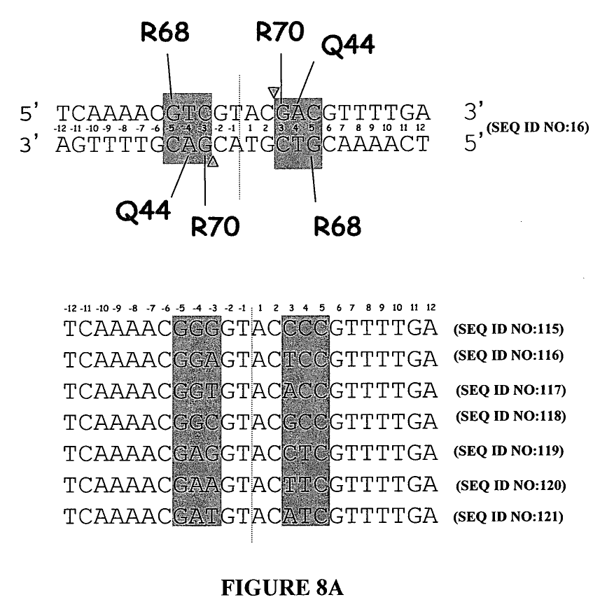

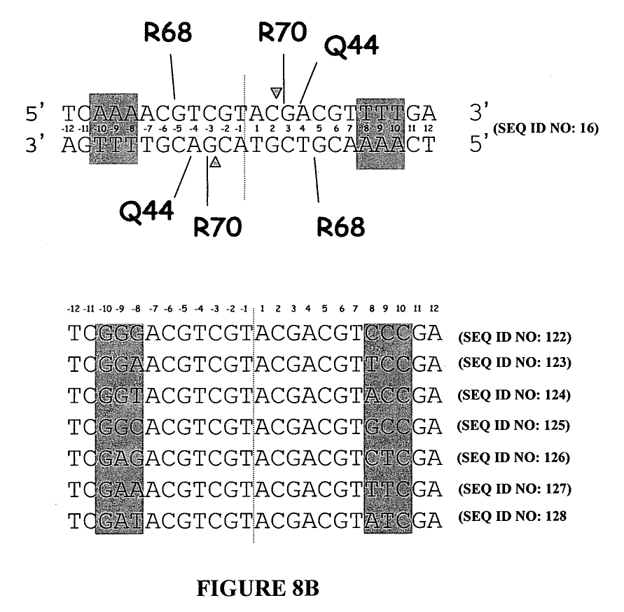

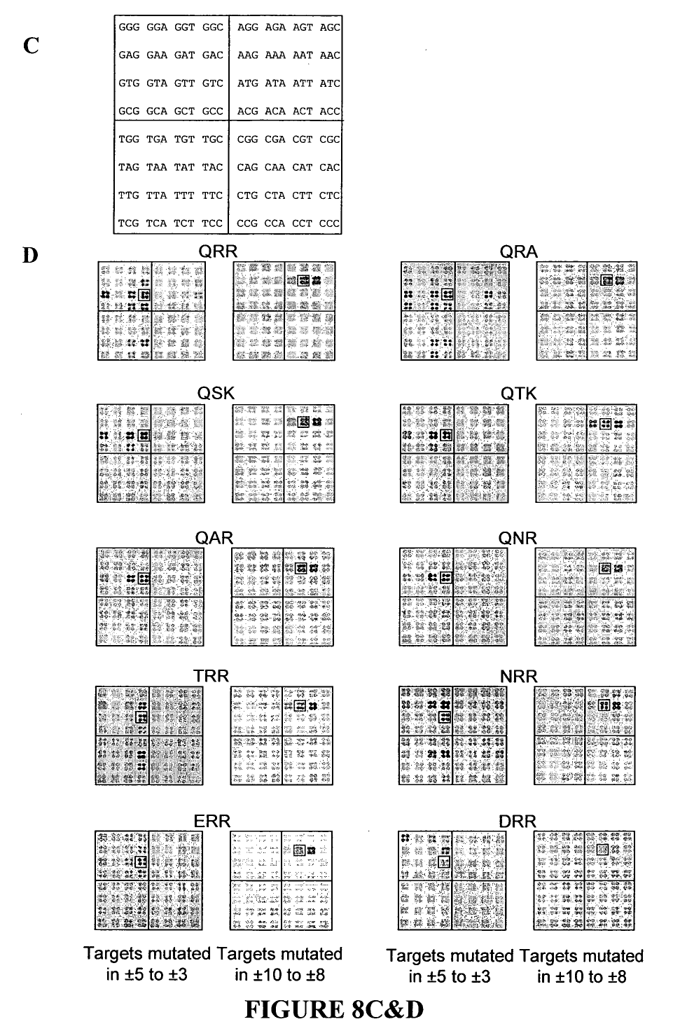

FIG. 5 illustrates examples of patterns and the numbers of mutants cleaving each target. A. Examples of profiling. Each novel endonuclease is profiled in yeast on a series of 64 palindromic targets, arrayed as in FIG. 5B, differing from the sequence C1221 (SEQ ID NO: 16; FIG. 8B), at positions ±8, ±9 and ±10. Each target sequence is named after the −10,−9,−8 triplet (10NNN). For example GGG corresponds to the tcgggacgtcgtacgacgtcccga target (SEQ ID NO:122; FIG. 8B). Meganucleases are tested 4 times against the 64 targets. Targets cleaved by I-CreI (D75), I-CreI N75 or ten derived variants are visualised by black or grey spots. B. Numbers of mutants cleaving each target, and average intensity of cleavage. Each sequence is named after the −10,−9,−8 triplet (10NNN). The number of proteins cleaving each target is shown below, and the level of grey coloration is proportional to the average signal intensity obtained with these cutters in yeast.

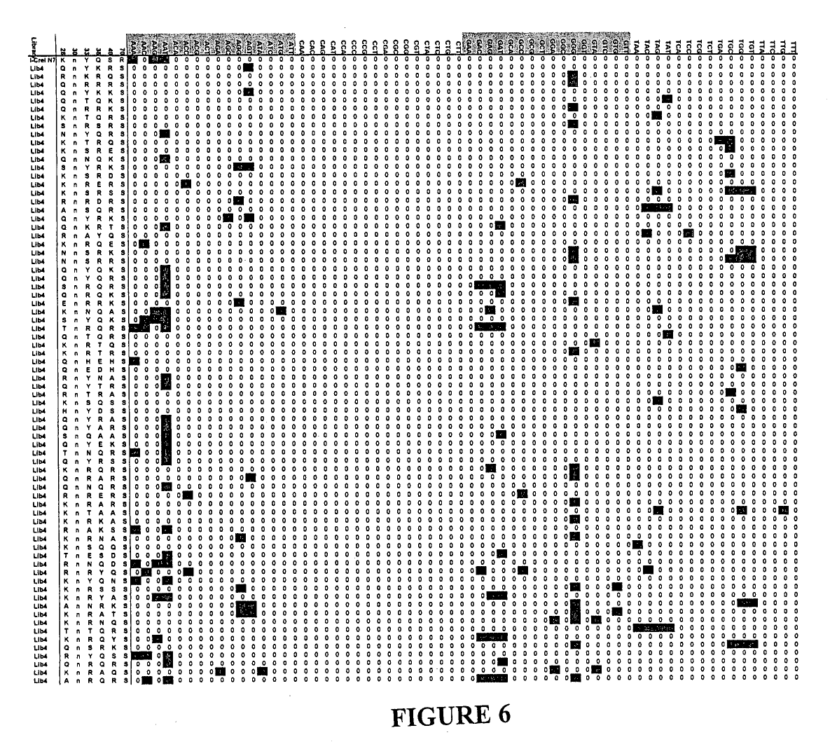

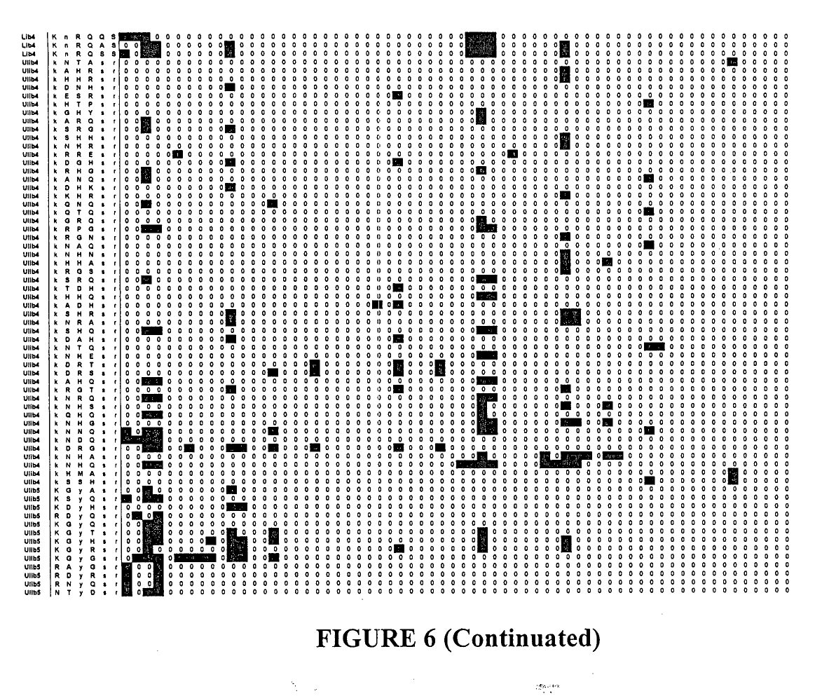

FIG. 6 represents the cleavage patterns of the I-CreI variants in position 28, 30, 33, 38 and/or 40. For each of the 141 I-CreI variants obtained after screening, and defined by residues in position 28, 30, 33, 38, 40, 70 and 75, cleavage was monitored in yeast with the 64 targets derived from the C1221 palindromic target cleaved by I-CreI, by substitution of the nucleotides in positions ±8 to 10.Targets are designated by three letters, corresponding to the nucleotides in position −10,−9 and −8. For example GGG corresponds to the tcgggacgtcgtacgacgtcccga target (SEQ ID NO: 122). Values (boxed) correspond to the intensity of the cleavage, evaluated by an appropriate software after scanning of the filter, whereas (0) indicates no cleavage.

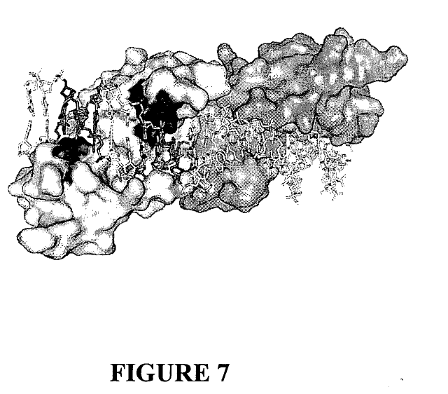

FIG. 7 represents the localisation of the mutations in the protein and DNA target, on a I-CreI homodimer bound to its target. The two set of mutations (residues 44, 68 and 70; residues 30, 33 and 38) are shown in black on the monomer on the left. The two sets of mutations are clearly distinct spatially. However, there is no structural evidence for distinct subdomains. Cognate regions in the DNA target site (region −5 to −3; region −10 to −8) are shown in grey on one half site.

FIG. 8: I-CreI derivative target definition (A and B) and profiling (C and D). All targets are derived from C1221, a palindromic target cleaved by I-CreI wild-type, and shown on the top of A and B. A. A first series of 64 targets is derived by mutagenesis of positions ±5 to ±3 (in grey boxes). A few examples are shown below. Interactions with I-CreI residues 44, 68 and 70 are shown. B. A second series of 64 target is derived by mutagenesis of positions ±10 to ±8 (in grey boxes). A few examples are shown below. Positions ±8, ±9 and ±10 are not contacted by residues 44, 68 and 70. C. Organisation of the targets as in FIG. 8D. For the left panels, the three letters in the table indicate the bases in positions −5, −4, −3 (for example, GGG means tcaaaacggggtaccccgttttga (SEQ ID NO: 115)). For the right panels, the three letters indicate the bases in positions −10, −9, −8 (for example, GGG means tcgggacgtcgtacgacgtcccga (SEQ ID NO: 122)). D. Profiling. Ten I-CreI variants cleaving the C1221 target, including I-CreI N75 (QRR) are profiled with the two sets of 64 targets (±5 to ±3 on the left, and ±10 to ±8 on the right). Targets are arranged as in FIG. 8C. The C1221 target (squared) is found in both sets. Mutants are identified by three letters corresponding to the residues found in position 44, 68 and 70 (example:QRR is Q44, R68, R70), and all of them have an additional D75N mutation.

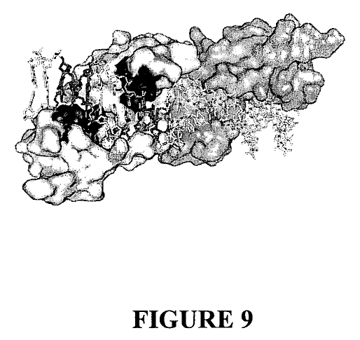

FIG. 9 represents the localisation of the mutations in the protein and DNA target, on a I-CreI homodimer bound to its target. The two set of mutations (residues 44, 68 and 70; residues 28, 30, 33, 38 and 40 are shown in black on the monomer on the left. The two sets of mutations are clearly distinct spatially. However, there is no structural evidence for distinct subdomains. Cognate regions in the DNA target site (region −5 to −3; region −10 to −8) are shown in grey on one half site.

FIG. 10 represents the HprCH3 series of targets and close derivatives. 10GAG_P, 10CAT_P and 5CTT_P (SEQ ID NO: 17 to 19) are close derivatives found to be cleaved by I-CreI mutants. They differ from C1221 (SEQ ID NO: 16) by the boxed motives. C1221, 10GAG_P, 10CAT_P and 5CTT_P were first described as 24 by sequences, but structural data suggest that only the 22 by are relevant for protein/DNA interaction. However, positions ±12 are indicated in parenthesis. In the HprCH3.2 target (SEQ ID NO: 20), the atga sequence in the middle of the target is replaced with gtac, the bases found in C1221. HprCH3.3 (SEQ ID NO: 21) is the palindromic sequence derived from the left part of HprCH3.2, and HprCH3.4 (SEQ ID NO: 22) is the palindromic sequence derived from the right part of HprCH3.2. As shown in the Figure, the boxed motives from 10GAG_P, 10CAT_P and 5CTT_P are found in the HprCH3 series of targets

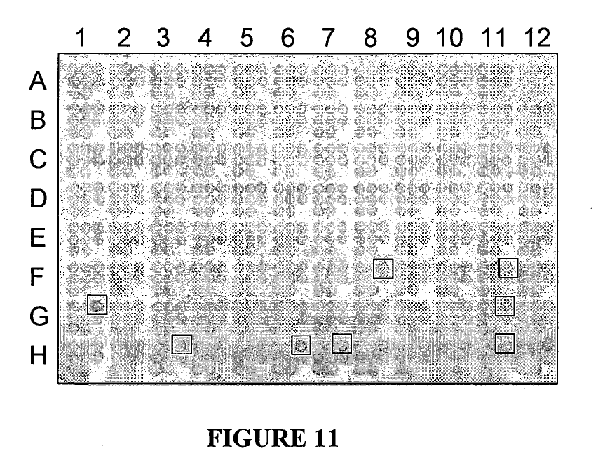

FIG. 11 illustrates cleavage of HprCH3.3 by 10NNN_P mutants. The figure displays an example of primary screening of I-CreI with the HprCH3.3 target. Positive clones are boxed. The sequences of positive mutants at position G1, H6 and H7 are KNDTQS/QRRDI (SEQ ID NO: 24), KNTPQS/QRRDI (SEQ ID NO: 44) and KNTTQS/QRRDI (SEQ ID NO: 45), respectively (same nomenclature as for Table III).

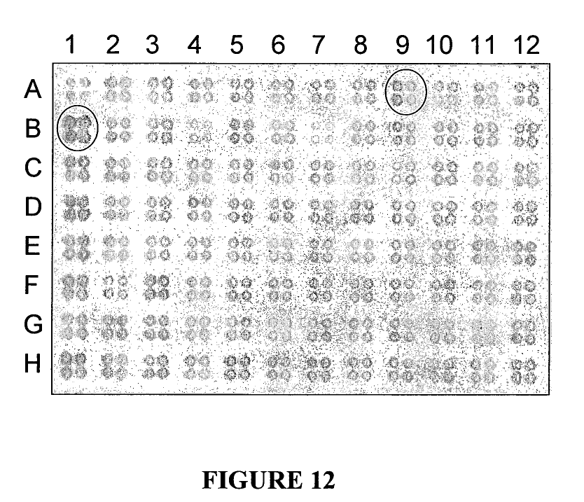

FIG. 12 illustrates cleavage of HprCH3.4 by combinatorial mutants. The figure displays an example of primary screening of I-CreI combinatorial mutants with the HprCH3.4 target. The sequences of positive mutants at position A9 and B1 are KNTHQS/RYSDN (SEQ ID NO: 54) and KNSYQS/RYSNI (SEQ ID NO: 60), respectively (same nomenclature as for Table IV).

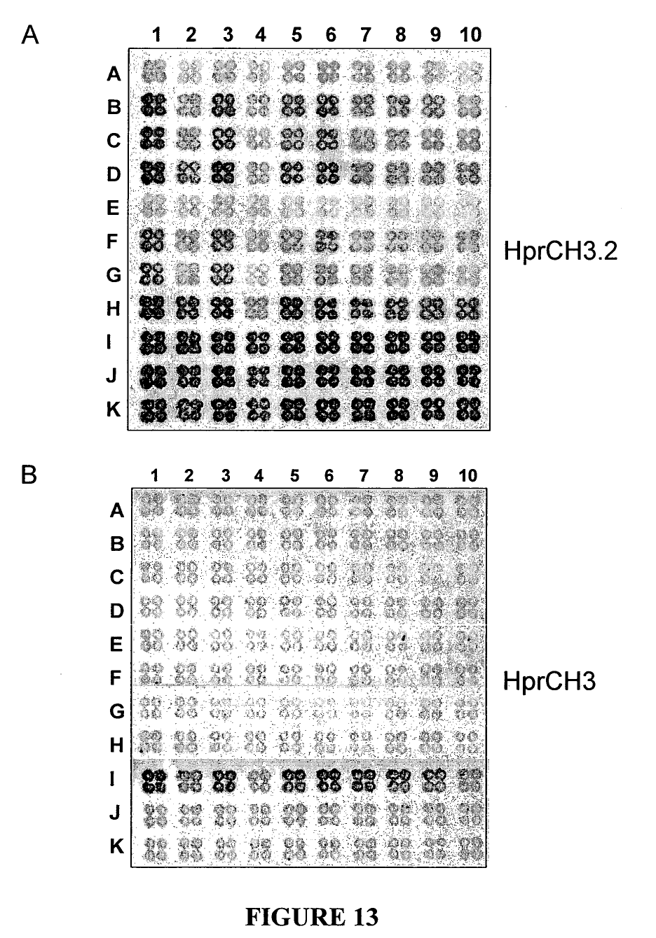

FIG. 13 illustrates cleavage of HprCH3.2 and HprCH3 by heterodimeric combinatorial mutants. A. Secondary screening of combinations of I-CreI mutants with the HprCH3.2. target. B. Secondary screening of the same combinations of I-CreI mutants with the HprCH3 target.

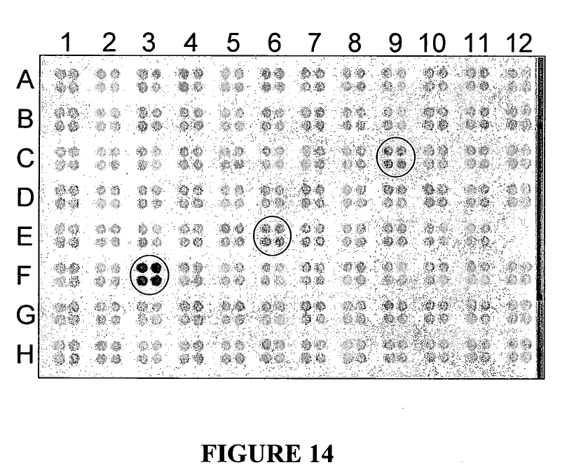

FIG. 14 illustrates cleavage of the HprCH3 target. A series of I-CreI mutants cutting HprCH3.4 were optimized and co-expressed with a mutant cutting HprCH3.3. Cleavage is tested with the HprCH3 target. Mutants displaying improved cleavage of HprCH3 are circled. In the filter shown, C9 corresponds to the heterodimer 28R,32S,33S,38Y,40Q,44R,68,70S,75N,77N (SEQ ID NO: 65)+33H (SEQ ID NO: 32), E6 corresponds to 28R,32S33S,38Y,40Q,44R,68A,70S,75H,77Y (SEQ ID NO: 66)+33H (SEQ ID NO: 32) and F3 corresponds to 28K,32T,33H,38Q,40S,44K,68Y,70S,75D,77R,92R,96R,107R,132V,140A,143A (SEQ ID NO:74)+33H (SEQ ID NO: 32). H11 is the original heterodimer (a mutant cleaving HprCH3.4, KSSQQS/RYSDN (SEQ ID NO:53), co-expressed with a mutant cleaving HprCH3.3, KNSHQS/QRRDI, (SEQ ID NO: 32). H12 is a positive control.

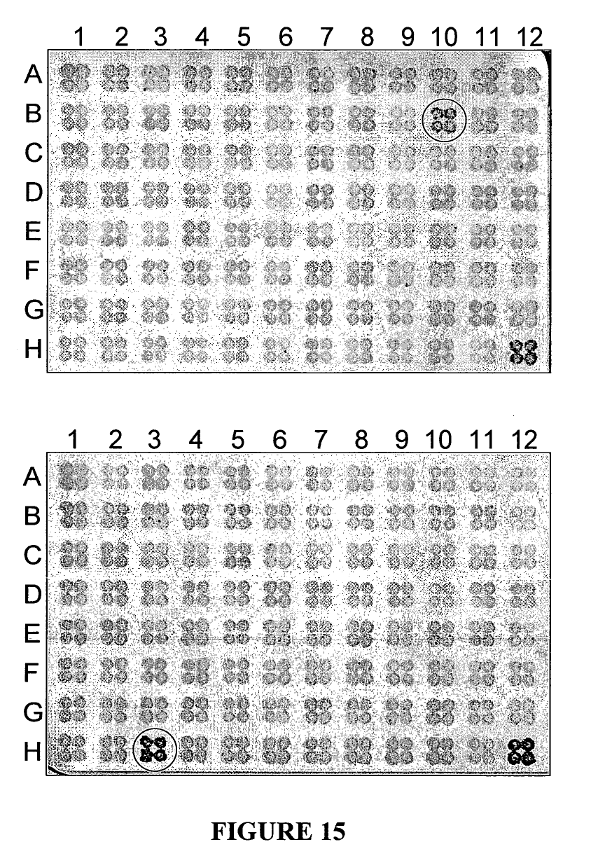

FIG. 15 illustrates cleavage of the HprCH3 target. A series of I-CreI mutants cutting HprCH3.3 were optimized and co-expressed with a mutant cutting HprCH3.4. Cleavage is tested with the HprCH3 target. Mutants displaying efficient cleavage of HprCH3 are circled. In the first filter, B 10 corresponds to the heterodimer 33H,71R,1031,129A and 130G (SEQ ID NO: 80)+33T,38Y,44K,68Y,70S,75E, and 77V (SEQ ID NO: 56). In the second filter, H3 corresponds to the heterodimer 21,33H,81V,861,110G,131R,135Q,151A and 157V (SEQ ID NO:79)+33T,38Y,44K,68Y,70S,75E and 77V (SEQ ID NO: 56). H12 is a positive control.

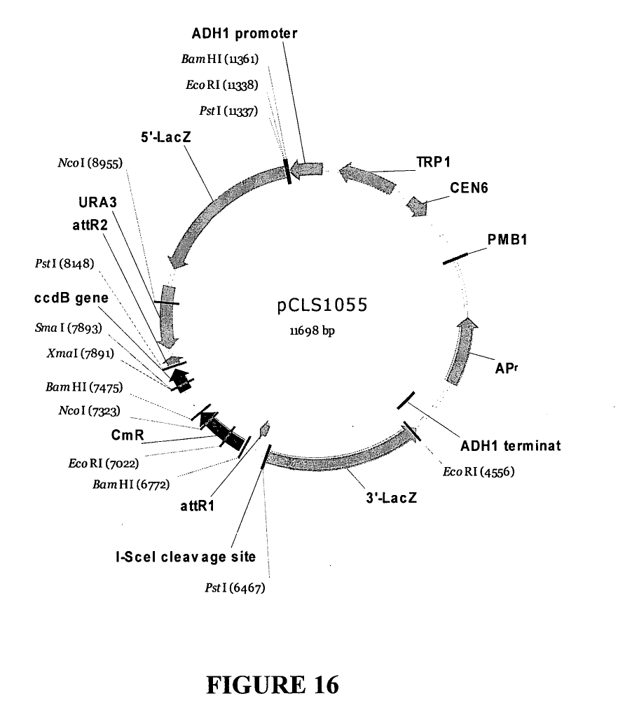

FIG. 16 represents the pCLS1055 vector map.

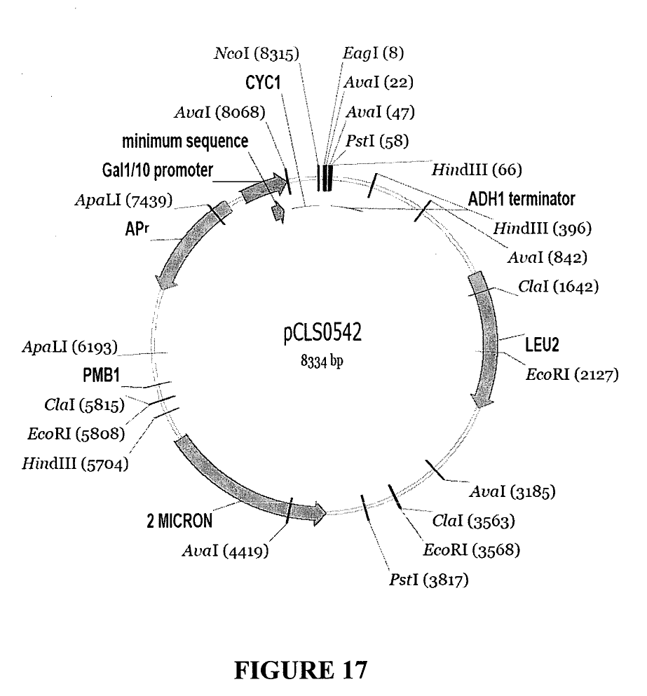

FIG. 17 represents the pCLS0542 vector map.

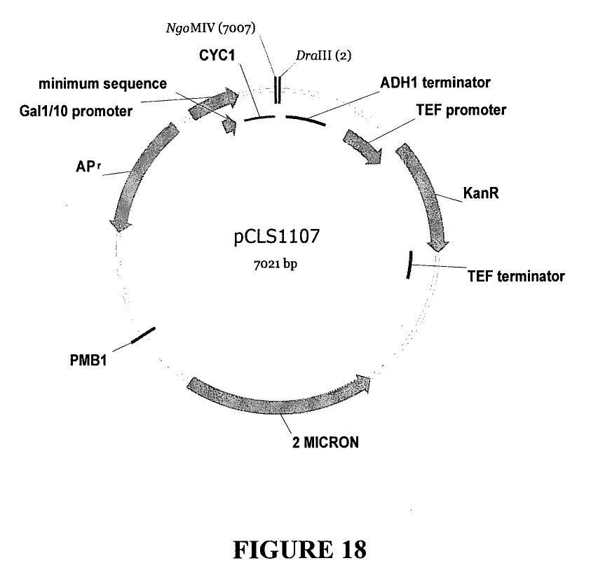

FIG. 18 represents the pCLS1107 vector map.

FIG. 19 illustrates the DNA target sequences which are present in the Criteculus griseus HPRT gene and the corresponding I-CreI variant which are able to cleave said DNA target. The DNA target is presented (column 3), with its first nucleotide (start, column 1) and last nucleotide (end, column 2); the positions are indicated relatively to the HPRT mRNA sequence (accession number J00060.1). The sequence of each heterodimeric variant is defined by the amino acid residues at the indicated positions of the first monomer (column 4) and the second monomer (column 5). For example, the first heterodimeric variant of FIG. 19 consists of a first monomer having K, Q, D, Y, Q, S, N, K, S, R and T in positions 28, 30, 32, 33, 38, 40, 44, 68, 70, 75 and 77, respectively and a second monomer having K, N, S, G, C, S, Q, R, R, N and I in positions 28, 30, 32, 38, 40, 44, 68, 70, 75 and 77, respectively. The positions are indicated by reference to I-CreI sequence SWISSPROT P05725 or pdb accession code 1g9y; I-CreI has K, N, S, Y, Q, S, Q, R, R, D, I, in positions 28, 30, 32, 33, 38, 40, 44, 68, 70, 75 and 77, respectively. The positions which are not indicated are not mutated and thus correspond to the wild-type I-CreI sequence.

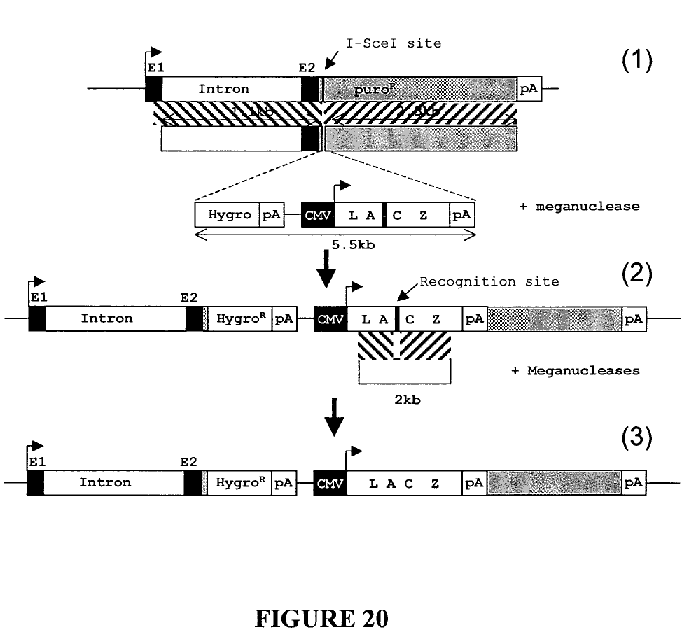

FIG. 20 illustrates the design of reporter system in mammalian cells. The puromycin resistance gene, interrupted by an I-SceI cleavage site 132 bp downstream of the start codon, is under the control of the EFIα promoter (1). The transgene has been stably expressed in CHO-K1 cells in single copy. In order to introduce Meganuclease target sites in the same chromosomal context, the repair matrix is composed of i) a promoterless hygromycin resistance gene, ii) a complete lacZ expression cassette and iii) two arms of homologous sequences (1.1 kb and 2.3 kb). Several repair matrixes have been constructed differing only by the recognition site that interrupts the lacZ gene (2). Thus, very similar cell lines have been produced as A1 cell line, I-SceI cell line and I-CreI cell line. A functional lacZ gene is restored when a lacZ repair matrix (2kb in length) is co-transfected with vectors expressing a meganuclease cleaving the recognition site (3). The level of meganuclease-induced recombination can be inferred from the number of blue colonies or foci after transfection.

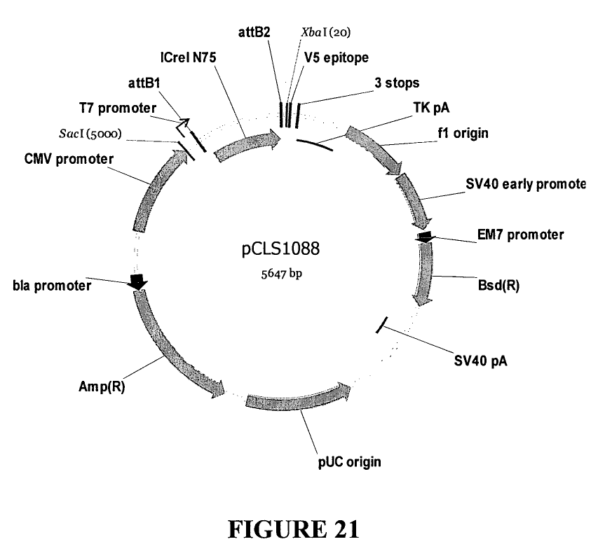

FIG. 21 represents the map of pCLS 1088, a plasmid for expression of I-CreI N75 in mammalian cells.

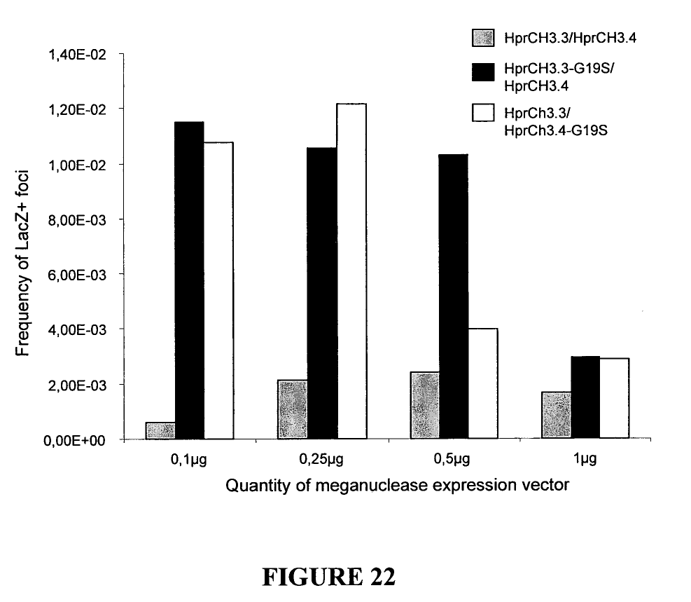

FIG. 22 illustrates cleavage efficiency of meganucleases cleaving the HprCH3 DNA target sequence. The frequency of repair of the LacZ gene is detected after transfection of CHO cells containing a HprCH3 chromosomal reporter system, with a repair matrix and various quantities of meganuclease expression vectors, coding for the initial engineered heterodimers (HprCH3.3/HprCH3.4) or their G19S derivatives (HprCH3.3/HprCh3.4 G19S or HprCH3.3 G19S/HprCh3.4).

### EXAMPLE 1

### Functional Endonucleases with New Specificity Towards Nucleotides ±8 to ±10 (10NNN)

The method for producing meganuclease variants and the assays based on cleavage-induced recombination in mammal or yeast cells, which are used for screening variants with altered specificity are described in the International PCT Application WO 2004/067736; Epinat et al., Nucleic Acids Res., 2003, 31, 2952-2962; Chames et al., Nucleic Acids Res., 2005, 33, e178, and Arnould et al., J. Mol. Biol., 2006, 355, 443-458. These assays result in a functional LacZ reporter gene which can be monitored by standard methods.

### A) Material and Methods

### a) Construction of Mutant Libraries

I-CreI wt (I-CreI D75), I-CreI D75N (I-CreI N75) and I-CreI S70 N75 open reading frames were synthesized, as described previously (Epinat et al., N.A.R., 2003, 31, 2952-2962). Combinatorial libraries were derived from the I-CreI N75, I-CreI D75 and I-CreI S70 N75 scaffolds, by replacing different combinations of residues, potentially involved in the interactions with the bases in positions ±8 to 10 of one DNA target half-site (Q26, K28, N30, S32, Y33, Q38 and S40). The diversity of the meganuclease libraries was generated by PCR using degenerated primers harboring a unique degenerated codon at each of the selected positions.

Mutation D75N was introduced by replacing codon 75 with aac. Then, the three codons at positions N30, Y33 and Q38 (Ulib4 library) or K28, N30 and Q38 (Ulib5 library) were replaced by a degenerated codon VVK (18 codons) coding for 12 different amino acids: A,D,E,G,H,K,N,P,Q,R,S,T). In consequence, the maximal (theoretical) diversity of these protein libraries was 12³ or 1728. However, in terms of nucleic acids, the diversity was 18³ or 5832.

In Lib4, ordered from BIOMETHODES, an arginine in position 70 of the I-CreI N75 scaffold was first replaced with a serine (R70S). Then positions 28, 33, 38 and 40 were randomized. The regular amino acids (K28, Y33, Q38 and S40) were replaced with one out of 10 amino acids (A,D,E,K,N,Q,R,S,T,Y). The resulting library has a theoretical complexity of 10000 in terms of proteins.

In addition, small libraries of complexity 225 (15²) resulting from the randomization of only two positions were constructed in an I-CreI N75 or I-CreI D75 scaffold, using NVK degenerate codon (24 codons, amino acids ACDEGHKNPQRSTWY).

Fragments carrying combinations of the desired mutations were obtained by PCR, using a pair of degenerated primers coding for 10, 12 or 15 different amino acids, and as DNA template, the I-CreI N75 (FIG. 4A), I-CreI D75 or I-CreI S70 N75 open reading frames (ORF). For example, FIG. 4B illustrates the two pair of primers (Ulib456for and Ulib4rev; Ulib456for and Ulib5rev) used to generate the Ulib4 and Ulib5 libraries, respectively. The corresponding PCR products were cloned back into the I-CreI N75, I-CreI D75 or I-CreI S70 N75 ORF, in the yeast replicative expression vector pCLS0542 (Epinat et al., precited; FIG. 17), carrying a LEU2 auxotrophic marker gene. In this 2 micron-based replicative vector, I-CreI variants are under the control of a galactose inducible promoter.

### b) Construction of Target Clones

The 64 palindromic targets derived from C1221 were constructed as described follows: 64 pairs of oligonucleotides (ggcatacaagtttcnnnacgtcgtacgacgtnnngacaatcgtctgtca (SEQ ID NO: 109) and reverse complementary sequenceswere ordered from Sigma, annealed and cloned into pGEM-T Easy (PROMEGA) in the same orientation. Next, a 400 by PvuII fragment was excised and cloned into the yeast vector pFL39-ADH-LACURAZ, also called pCLS0042, described previously (Epinat et al., precited), resulting in 64 yeast reporter vectors (target plasmids).

### c) Yeast Strains

The three libraries of meganuclease expression variants were transformed into the leu2 mutant haploid yeast strain FYC2-6A: MATalpha, trp1Δ63, leu2Δ1, his3Δ200. A classical chemical/heat choc protocol that routinely gives us 10⁶ independent transformants per μg of DNA derived from (Gietz and Woods, Methods Enzymol., 2002, 350, 87-96), was used for transformation. Individual transformant (Leu⁺) clones were individually picked in 96 wells microplates. The 64 target plasmids were transformed using the same protocol, into the haploid yeast strain FYBL2-7B: MATα, ura3Δ851, trp1Δ63, leu2Δ1, lys2Δ202, resulting in 64 tester strains.

### d) Mating of Meganuclease Expressing Clones and Screening in Yeast

Meganuclease expressing clones were mated with each of the 64 target strains, and diploids were tested for beta-galactosidase activity, by using the screening assay previously described in the International PCT Application WO 2004/067736; Epinat et al., Nucleic Acids Res., 2003, 31, 2952-2962; Chames et al., Nucleic Acids Res., 2005, 33, e178, and Arnould et al., J. Mol. Biol., 2006, 355, 443-458. I-CreI variant clones as well as yeast reporter strains were stocked in glycerol (20%) and replicated in novel microplates. Mating was performed using a colony gridder (QpixII, GENETIX). Mutants were gridded on nylon filters covering YPD plates, using a high density (about 20 spots/cm²). A second gridding process was performed on the same filters to spot a second layer consisting of 64 different reporter-harboring yeast strains for each variant. Membranes were placed on solid agarose YEPD rich medium, and incubated at 30° C. for one night, to allow mating. Next, filters were transferred to synthetic medium, lacking leucine and tryptophan, with galactose (1%) as a carbon source (and with G418 for coexpression experiments), and incubated for five days at 37° C., to select for diploids carrying the expression and target vectors. After 5 days, filters were placed on solid agarose medium with 0.02% X-Gal in 0.5 M sodium phosphate buffer, pH 7.0, 0.1% SDS, 6% dimethyl formamide (DMF), 7 mM β-mercaptoethanol, 1% agarose, and incubated at 37° C., to monitor β-galactosidase activity. After two days of incubation, positive clones were identified by scanning and the β-galactosidase activity of the clones was quantified using an appropriate software.

The clones showing an activity against at least one target were isolated (first screening) and each positive clone was tested against the 64 reporter strains in quadruplicate, thereby creating complete profiles (secondary screening).

### c) Sequence

The open reading frame (ORF) of positive clones identified during the first and/or secondary screening in yeast was amplified by PCR on yeast colonies using primers: PCR-Ga110-F (gcaactttagtgctgacacatacagg, SEQ ID NO: 48) and PCR-Ga110-R (acaaccttgattgcagacttgacc, SEQ ID NO: 49)from PROLIGO. Briefly, yeast colony is picked and resuspended in 100 μl of LGlu liquid medium and cultures overnight. After centrifugation, yeast pellet is resuspended in 10 μl of sterile water and used to perform PCR reaction in a final volume of 50 μl containing 1.5 μl of each specific primers (100 pmol/μl). The PCR conditions were one cycle of denaturation for 10 minutes at 94° C., 35 cycles of denaturation for 30 s at 94° C., annealing for 1 min at 55° C., extension for 1.5 min at 72° C., and a final extension for 5 min. Sequencing was performed directly on the PCR product by MILLEGEN.

### d) Structure Analyses

All analyses of protein structures were realized using Pymol. The structures from I-CreI correspond to pdb entry 1g9y. Residue numbering in the text always refer to these structures, except for residues in the second I-CreI protein domain of the homodimer where residue numbers were set as for the first domain.

### B) Results

I-CreI is a dimeric homing endonuclease that cleaves a 22 by pseudo-palindromic target. Analysis of I-CreI structure bound to its natural target has shown that in each monomer, eight residues establish direct interactions with seven bases (Jurica et al., 1998, precited). According to these structural data, the bases of the nucleotides in positions ±8 to 10 establish direct contacts with I-CreI amino-acids N30, Y33, Q38 and indirect contacts with I-CreI amino-acids K28 and S40. Thus, novel proteins with mutations in positions 30, 33 and 38 could display novel cleavage profiles with the 64 targets resulting from substitutions in positions ±8, ±9 and ±10 of a palindromic target cleaved by I-CreI (10NNN target). In addition, mutations might alter the number and positions of the residues involved in direct contact with the DNA bases. More specifically, positions other than 30, 33, 38, but located in the close vicinity on the folded protein, could be involved in the interaction with the same base pairs.

An exhaustive protein library vs. target library approach was undertaken to engineer locally this part of the DNA binding interface. Randomization of 5 amino acids positions would lead to a theoretical diversity of 20⁵=3.2×10⁶. However, libraries with lower diversity were generated by randomizing 2, 3 or 4 residues at a time, resulting in a diversity of 225 (15²), 1728 (12³) or 10,000 (10⁴). This strategy allowed an extensive screening of each of these libraries against the 64 palindromic 10NNN DNA targets using a yeast based assay described previously (Epinat et al., 2003, precited and International PCT Application WO 2004/067736).

First, the I-CreI scaffold was mutated from D75 to N. The D75N mutation did not affect the protein structure, but decreased the toxicity of I-CreI in overexpression experiments.

Next the Ulib4 library was constructed : residues 30, 33 and 38, were randomized, and the regular amino acids (N30, Y33, and Q38) replaced with one out of 12 amino acids (A,D,E,G,H,K,N,P,Q,R,S,T). The resulting library has a complexity of 1728 in terms of protein (5832 in terms of nucleic acids).

Then, two other libraries were constructed : Ulib5 and Lib4. In Ulib5, residues 28, 30 and 38, were randomized, and the regular amino acids (K28, N30, and Q38) replaced with one out of 12 amino acids (ADEGHKNPQRST). The resulting library has a complexity of 1728 in terms of protein (5832 in terms of nucleic acids). In Lib4, an Arginine in position 70 was first replaced with a Serine. Then, positions 28, 33, 38 and 40 were randomized, and the regular amino acids (K28, Y33, Q38 and S40) replaced with one out of 10 amino acids (A,D,E,K,N,Q,R,S,T,Y). The resulting library has a complexity of 10000 in terms of proteins.

In a primary screening experiment, 20000 clones from Ulib4, 10000 clones from Ulib5 and 20000 clones from Lib4 were mated with each one of the 64 tester strains, and diploids were tested for beta-galactosidase activity. All clones displaying cleavage activity with at least one out of the 64 targets were tested in a second round of screening against the 64 targets, in quadriplate, and each cleavage profile was established, as shown on FIG. 5. Then, meganuclease ORFs were amplified from each strain by PCR, and sequenced.

After secondary screening and sequencing of positives over the entire coding region, a total of 1484 unique mutants were isolated showing a cleavage activity against at least one target. Different patterns could be observed. FIG. 6 illustrates 37 novel targets cleaved by a collection of 141 variants, including 34 targets which are not cleaved by I-CreI and 3 targets which are cleaved by I-CreI (aag, aat and aac). Twelve examples of profile, including I-CreI N75 and I-CreI D75 are shown on FIG. 5A. Some of these new profiles shared some similarity with the wild type scaffold whereas many others were totally different. Homing endonucleases can usually accommodate some degeneracy in their target sequences, and the I-CreI and I-CreI N75 proteins themselves cleave a series of sixteen and three targets, respectively. Cleavage degeneracy was found for many of the novel endonucleases, with an average of 9.9 cleaved targets per mutant (standard deviation: 11). However, among the 1484 mutants identified, 219 (15%) were found to cleave only one DNA target, 179 (12%) cleave two, and 169 (11%) and 120 (8%) were able to cleave 3 and 4 targets respectively. Thus, irrespective of their preferred target, a significant number of I-CreI derivatives display a specificity level that is similar if not higher than that of the I-CreI N75 mutant (three 10NNN target sequences cleaved), or I-CreI (sixteen 10NNN target sequences cleaved). Also, the majority of the mutants isolated for altered specificity for 10NNN sequences no longer cleave the original C1221 target sequence (61% and 59%, respectively).

Altogether, this large collection of mutants allowed the targeting of all of the 64 possible DNA sequences differing at positions ±10, ±9, and ±8 (FIG. 5B). However, there were huge variations in the numbers of mutants cleaving each target (FIG. 5B), these numbers ranged from 3 to 936, with an average of 228.5 (standard deviation: 201.5). Cleavage was frequently observed for targets with a guanine in ±8 or an adenine in ±9, whereas a cytosine in ±10 or ±8 was correlated with low numbers of cleavers. In addition, all targets were not cleaved with the same efficiency. Since significant variations of signal could be observed for a same target, depending on the mutant (compare cleavage efficiencies for the wild type 10AAA target in FIG. 5B, for example), an average cleavage efficiency was measured for each target as previously reported (Arnould et al., J. Mol. Biol., 2006, 355, 443-458). These average efficiencies are represented by grey levels on FIG. 5B. Analysis of the results show a clear correlation between this average efficiency and the numbers of cleavers, with the most frequently cut target being also the most efficiently cut (compare for example 10TCN, 10CTN and 10CCN targets with 10GAN, 10AAN and 10TAN in FIG. 5B).

Thus, hundreds of novel variants were obtained, including mutants with novel substrate specificity ; these variants can keep high levels of activity and the specificity of the novel proteins can be even narrower than that of the wild-type protein for its target.

### EXAMPLE 2

### Two I-CreI Functional Subdomains can Behave Independently in Terms of DNA Binding

This example shows that an I-CreI target can be separated in two parts, bound by different subdomains, behaving independently. In the I-CreI DNA target, positions ±5, ±4 and ±3 are bound by residues 44, 68 and 70. Several I-CreI variants, mutated in positions 44, 68, 70 and 75, obtained as described in example 1, were shown to display a detectable activity on C1221, a palindromic target cleaved by I-CreI wild-type (Chevalier, et al., 2003), but were cleaving other targets with various efficacies. In the external part of the binding site, positions ±9 and ±8 are contacted by residues 30, 33 and 38. A shown on FIG. 7, the two set of residues are in distinct parts of the proteins. There is no direct interaction with bases ±8. If positions ±5 to ±3 and ±10 to ±8 are bound by two different, independent functional subdomains, engineering of one subdomain should not impact the binding properties of the other domain.

In order to determine if positions ±5 to ±3 and ±9 to ±8 are bound by two different, independent functional subdomains, mutants with altered specificity in the ±5 to ±3 region, but still binding C1221, were assayed for their cleavage properties in the ±10 to ±8 region.

### A) Material and Methods

### a) Structure Analyses

The experimental procedure is as in example 1.

### b) I-CreI Variant Expressing Yeast Strain

Mutants were generated as described in examples 1, by mutating positions 44, 68, 70 and 75, and screening for clones able to cleave C1221 derived targets. Mutant expressing plasmids are transformed into S. cerevisiae strain FYC2-6A (MATα, trp1Δ63, leu2Δ1, his3{200).

### c) Construction of Target Clone

The 64 palindromic target plasmids derived from C 1221 by mutation in ±5 to ±3 were constructed as described in example 1, by using 64 pairs of oligonucleotides (ggcatacaagtttcaaaacnnngtacnnngttttgacaatcgtctgtca (SEQ ID NO: 110) and reverse complementary sequences). The 64 target plasmids were transformed using the protocol described in example 1, into the haploid yeast strain FYBL2-7B: MATa, ura3Δ851, trp1Δ63, leu2Δ1, lys2Δ202, resulting in 64 tester strains.

### d) Mating of Meganuclease Expressing Clones and Screening in Yeast

Mating was performed as described in example 1, using a low gridding density (about 4 spots/cm²).

### B) Results

64 targets corresponding to all possible palindromic targets derived from C1221 were constructed by mutagenesis of bases ±10 to ±8, as shown on FIG. 8B. The I-CreI N75 cleavage profile was established, showing a strong signal with the aaa and aat targets, and a weaker one with the aag target.

As shown on FIG. 8C, proteins with a clearly different cleavage profile in ±5 to ±3, such as QAR, QNR, TRR, NRR, ERR and DRR have a similar profile in ±10 to ±8. The aaa sequence in ±10 to ±8 corresponds to the C1221 target, and is necessarily cleaved by all our variants cleaving C 1221. aat is cleaved as well in most mutants (90%), whereas aag is often not observed, probably because the signal drops below the detection level in faint cleaver. No other target is ever cleaved. These results show that the ±5 to ±3 and ±10 to regions are bound by two different, largely independent binding units.

### EXAMPLE 3

### Strategy for Engineering Novel Meganucleases Cleaving a Target from the HPRT Gene

A) Principle of the Combinatorial Approach for Designing Novel Meganucleases with Tailored Specificity

The objective here is to determine whether it is possible to combine separable functional subdomains in the I-CreI DNA-binding interface, in order to cleave novel DNA targets.

The identification of distinct groups of mutations in the I-CreI coding sequence that alter the cleavage specificity towards two different regions of the C 1221 target sequence (10NNN (positions −10 to −8 and +8 to +10: ±8 to 10 or ±10 to 8; example 1) and 5NNN (positions-5 to −3 and +3 to +5: ±3 to 5 or ±5 to 3; Arnould et al., J. Mol. Biol., 2006, 355, 443-458, International Applications WO 2006/097784 and WO 2006/097853) raises the possibility of combining these two groups of mutants intramolecularly to generate a combinatorial mutant capable of cleaving a target sequence simultaneously altered at positions 10NNN and 5NNN (FIG. 1C).

Positions 28, 30, 33, 38 and 40 on one hand, and 44, 68 and 70, on another hand are on a same DNA-binding fold, and there is no structural evidence that they should behave independently. However, the two sets of mutations are clearly on two spatially distinct regions of this fold (FIGS. 7 and 9) located around different regions of the DNA target. In addition, the cumulative impact of a series of mutations could eventually disrupt the folding. To check whether they are part of two independent functional subunits, mutations from these two series of mutants were combined, and the ability of the resulting variants to cleave the combined target sequence was assayed (FIG. 1D).

Therefore, a non-palindromic target sequence that would be a patchwork of four cleaved 5NNN and 10NNN targets, is identified. In addition, two derived target sequences representing the left and right halves in palindromic form, are designed. To generate appropriate I-CreI combinatorial mutants capable of targeting the palindromic targets, mutants efficiently cleaving the 10NNN and 5NNN part of each palindromic sequence are selected and their characteristic mutations incorporated into the same coding sequence by in vivo cloning in yeast.

Throughout the text and figures, combinatorial mutants sequences are named with an eleven letter code, after residues at positions 28, 30, 32, 33, 38, 40, 44, 68 and 70, 75 and 77. For example, KNSTYS/KYSEV stands for I-CreI K28, N30, S32, T33, Y38, S40, K44, Y68, S70, E75, and V77 (I-CreI 28K, 30N, 32S, 33T, 38Y, 40S, 44K, 68Y, 70S, 75E and 77V). Parental controls are named with a six letter code, after residues at positions 28, 30, 32, 33, 38 and 40 or a five letter code, after residues at positions 44, 68, 70, 75 and 77. For example, KNSTYS stands for I-CreI 28K, 30N, 32S, 33T, 38Y and 40S, and KYSEV stands for -CreI 44K, 68Y, 70S, 75E and 77V.

All target sequences described in these examples are 22 or 24 by palindromic sequences. Therefore, they will be described only by the first 11 or 12 nucleotides, followed by the suffix _P; for example, target 5′ tcaaaacgtcgtacgacgttttga 3′ (SEQ ID NO:16) cleaved by the I-CreI protein, will be called tcaaaacgtcgt_P.

B) Design of Novel Meganucleases Cleaving a Target from the Criteculus griseus HPRT Gene

This combinatorial approach, was used to engineer the DNA binding domain of the I-CreI meganuclease, and cleave the Cricetulus griseus HPRT gene.

HprCH3 is a 22 by (non-palindromic) target (FIG. 2) located in Exon 3 (positions 17 to 38) of the Criteculus griseus (Chinese Hamster) HPRT gene; the target sequence corresponds to positions 241 to 262 of the mRNA (accession number J00060; SEQ ID NO: 15; FIG. 2).

The meganucleases cleaving HprCH3 could be used, either to insert an heterologous gene of interest at the HPRT locus, to allow reproducible gene expression levels in vertebrate recombinant cell lines or transgenic animals, or to inactivate the HPRT gene, to allow the selection of vertebrate recombinant cell lines or transgenic animals (FIGS. 3A and 3B).

The HprCH3 sequence is partly a patchwork of the 10GAG_P, 10CAT P and 5CTT_{—} P targets (FIG. 10) which are cleaved by previously identified meganucleases, obtained as described in International PCT Applications WO 2006/097784 and WO 2006/097853; Arnould et al., J. Mol. Biol., 2006, 355, 443-458; example 1. Thus, HprCH3 could be cleaved by combinatorial mutants resulting from these previously identified meganucleases.

The 10GAG_P, 10CAT_P and 5CTT_P target sequences are 24 by derivatives of C1221, a palindromic sequence cleaved by I-CreI (Arnould et al., precited). However, the structure of I-CreI bound to its DNA target suggests that the two external base pairs of these targets (positions −12 and 12) have no impact on binding and cleavage (Chevalier et al., Nat. Struct. Biol., 2001, 8, 312-316; Chevalier and Stoddard, Nucleic Acids Res., 2001, 29, 3757-3774; Chevalier et al., J. Mol. Biol., 2003, 329, 253-269), and in this study, only positions −11 to 11 were considered. Consequently, the HprCH3 series of targets were defined as 22 by sequences instead of 24 bp. HprCH3 differs from C1221 in the 4 by central region. According to the structure of the I-CreI protein bound to its target, there is no contact between the 4 central base pairs (positions −2 to 2) and the I-CreI protein (Chevalier et al., Nat. Struct. Biol., 2001, 8, 312-316; Chevalier and Stoddard, Nucleic Acids Res., 2001, 29, 3757-3774; Chevalier et al., J. Mol. Biol., 2003, 329, 253-269). Thus, the bases at these positions should not impact the binding efficiency. However, they could affect cleavage, which results from two nicks at the edge of this region. Thus, the atga sequence in -2 to 2 was first substituted with the gtac sequence from C1221, resulting in target HprCH3.2 (FIG. 10). Then, two palindromic targets, HprCH3.3 and HprCH3.4, were derived from HprCH3.2 (FIG. 10). Since HprCH3.3 and HprCH3.4 are palindromic, they should be cleaved by homodimeric proteins. Thus, proteins able to cleave the HprCH3.3 and HprCH3.4 sequences as homodimers were first designed (examples 4 and 5) and then co-expressed to obtain heterodimers cleaving HprCH3 (example 6). Heterodimers cleaving the HprCH3.2 and HprCH3 targets could be identified. In order to improve cleavage activity for the HprCH3 target, a series of mutants cleaving HprCH3.3 and HprCH3.4 was chosen, and then refined. The chosen mutants were randomly mutagenized, and used to form novel heterodimers that were screened against the HprCH3 target (examples 7 and 8). Heterodimers could be identified with an improved cleavage activity for the HprCH3 target.

### EXAMPLE 4

### Identification of Meganucleases Cleaving HprCH3.3

This example, shows that I-CreI mutants can cut the HprCH3.3 DNA target sequence derived from the left part of the HprCH3.2 target in a palindromic form (FIG. 10). Target sequences described in this example are 22 by palindromic sequences. Therefore, they will be described only by the first 11 nucleotides, followed by the suffix_P (For example, target HprCH3.3 will be noted cgagatgtcgt_P (SEQ ID NO: 21). HprCH3.3 is similar to 10GAG_P at all positions except ±6. It was hypothesized that positions ±6 would have little effect on the binding and cleavage activity. Mutants able to cleave the 10GAG_P target were obtained by mutagenesis of I-CreI or I-CreI S70 N75, at positions 28, 30, 32, 33, 38, 40, as described in example 1. Screening of these mutants would allow the identification of meganucleases that cleave the HprCH3.3 target.

### A) Material and Methods

### a) Construction of Target Vector

The target was cloned as follows: oligonucleotide corresponding to the target sequence flanked by gateway cloning sequence was ordered from PROLIGO: 5′tggcatacaagtttcgagatgtcgtacgacatctcgacaatcgtctgtca3′ (SEQ ID NO: 23). Double-stranded target DNA, generated by PCR amplification of the single stranded oligonucleotide, was cloned using the Gateway protocol (INVITROGEN) into the yeast reporter vector (pCLS 1055, FIG. 16). Yeast reporter vector was transformed into Saccharomyces cerevisiae strain FYBL2-7B (MAT a, ura3Δ851, trp1Δ63, leu2Δ1, lys2Δ202), resulting in a reporter strain.

### b) Mating of Meganuclease Expressing Clones and Screening in Yeast

I-CreI mutants cleaving 10GAG_P were previously identified, as described in example 1. These mutants were present on a yeast expression plasmid (pCLS0542, FIG. 17) in the S. cerevisiae strain FYC2-6A (MATα, trp1Δ63, leu2Δ1, his3Δ200).

Meganuclease expressing clones were mated with the reporter strain and diploids were tested for beta-galactosidase activity, by using the screening assay as described in example 1, using a low gridding density (about 4 spots/cm²).

### c) Sequencing of Mutants

The experimental procedure is as described in example 1.

### B) Results

I-CreI mutants capable of cleaving 10GAG_P were screened for cleavage against the HprCH3.3 DNA target (cgagatgtcgt_P; (SEQ ID NO: 21). 38 positives clones were found, and after sequencing and validation by secondary screening, 24 mutants listed in Table III were identified. Examples of positives are shown in FIG. 11.

TABLE III
CreI mutants capable of cleaving the HprCH3.3
DNA target.

| Amino acids at positions 28, 30, 32, | |
|---|---|
| 33, 38, 40/44, 68, 70, 75 and 77 | |
| of the I-CreI mutants | |
| (ex: KNDTQS/QRRDI stands for K28, | |
| N30, D32, T33, Q38, S40/Q44, R68, | SEQ ID |
| R70, D75 and I77) | NO: |
| KNDTQS/QRRDI | 24 |
| KNETQS/QRRDI | 25 |
| KNPAQS/QRRDI | 26 |
| KNRDQS/QRRDI | 27 |
| KNSCKS/QRRDI | 28 |
| KNSCSS/QRRDI | 29 |
| KNSCTS/QRRDI | 30 |
| KNSGAS/QRRDI | 31 |
| KNSHQS/QRRDI | 32 |
| KNSPHS/QRRDI | 33 |
| KNSPQS/QRRDI | 34 |
| KNSQQS/QRRDI | 35 |
| KNSQYS/QRRDI | 36 |
| KNSRQY/QRSNI | 37 |
| KNSSDS/QRRDI | 38 |
| KNSTGS/QRRDI | 39 |
| KNSTNS/QRRDI | 40 |
| KNSTSS/QRRDI | 41 |
| KNSTTS/QRRDI | 42 |
| KNSVHS/QRRDI | 43 |
| KNTPQS/QRRDI | 44 |
| KNTTQS/QRRDI | 45 |
| KTSTNS/QRRDI | 46 |
| TNSRQR/QRSNI | 47 |

### EXAMPLE 5

### Making of Meganucleases Cleaving HprCH3.4

This example shows that I-CreI mutants can cleave the HprCH3.4 DNA target sequence derived from the right part of the HprCH3.2 target in a palindromic form (FIG. 10). All target sequences described in this example are 22 by palindromic sequences. Therefore, they will be described only by the first 11 nucleotides, followed by the suffix_P (for example, HprCH3.4 will be called ccatctcttgt_P; SEQ ID NO: 22).

HprCH3.4 is similar to 5CTT_P at positions ±1, ±2, ±3, ±4, ±5 and ±11 and to 10CAT_P at positions ±1, ±2, ±8, ±9 ±10 and ±11. It was hypothesized that positions ±6 and ±7 would have little effect on the binding and cleavage activity. Mutants able to cleave 5CTT_P (caaaaccttgt_P; SEQ ID NO: 19) were obtained by mutagenesis of I-CreI N75 at positions 44, 68 and 70 or I-CreI S70 at positions 44, 68, 75 and 77, as described previously (International PCT Applications WO 2006/097784 and WO 2006/097853; Arnould et al., J. Mol. Biol., 2006, 355, 443-458). Mutants able to cleave the 10CAT_P target (ccatacgtcgt_P; SEQ ID NO: 18) were obtained by mutagenesis of I-CreI (D75), at positions 30, 32, 33 and 38, as described in example 1. Thus, combining such pairs of mutants would allow for the cleavage of the HprCH3.4 target. Therefore, to check whether combined mutants could cleave the HprCH3.4 target, amino acids at positions 44, 68, 70, 75 and 77 from proteins cleaving 5CTT_P were combined with the amino acids at positions 30, 32, 33 and 38 from proteins cleaving 10CAT_P.

### A) Material and Methods

### a) Construction of Target Vector

The experimental procedure is as described in example 4.

### b) Construction of Combinatorial Mutants

I-CreI mutants cleaving 10CAT_P or 5CTT_P were previously identified, as described in International PCT Applications WO 2006/097784 and WO 2006/097853; Arnould et al., J. Mol. Biol., 2006, 355, 443-458, and example 1. In order to generate I-CreI derived coding sequence containing mutations from both series, separate overlapping PCR reactions were carried out that amplify the 5′ end (aa positions 1-43) or the 3′ end (positions 39-167) of the I-CreI coding sequence. For both the 5′ and 3′ end, PCR amplification is carried out using primers (Gal10F 5′-gcaactttagtgctgacacatacagg-3′ (SEQ ID NO: 48) or Gal10R 5′-acaaccttgattggagacttgacc-3′ (SEQ ID NO: 49) specific to the vector (pCLS0542, FIG. 11) and primers (assF 5′-ctannnttgaccttt-3′ (SEQ ID NO: 50) or assR 5′-aaaggtcaannntag-3′(SEQ ID NO: 51), where nnn codes for residue 40. The PCR fragments resulting from the amplification reaction realized with the same primers and with the same coding sequence for residue 40 were pooled. Then, each pool of PCR fragments resulting from the reaction with primers Gal10F and assR or assF and Gal10R was mixed in an equimolar ratio. Finally, approximately 25 ng of each final pool of the two overlapping PCR fragments and 75 ng of vector DNA (pCLS1107, FIG. 18) linearized by digestion with DraIII and NgoMIV were used to transform the yeast Saccharomyces cerevisiae strain FYC2-6A (MATα, trp1Δ63, leu2Δ1, his3Δ200) using a high efficiency LiAc transformation protocol (Gietz and Woods, Methods Enzymol., 2002, 350, 87-96). An intact coding sequence containing both groups of mutations is generated by in vivo homologous recombination in yeast.

### c) Mating of Meganuclease Expressing Clones and Screening in Yeast

The experimental procedure is as described in example 4

### d) Sequencing of Mutants

The experimental procedure is as described in example 4

### B) Results

I-CreI mutants used in this example, and cutting the 10CAT_P target or the 5CTT_P target are listed in Table IV. I-CreI combined mutants were constructed by associating on the I-CreI scaffold, amino acids at positions 44, 68, 70, 75 and 77 from mutants cleaving the 5CTT_P target, with the amino acids at positions 30, 32, 33 and 38 from the mutants cleaving the 10CAT_P target (Table IV), resulting in a library of complexity 480. This library was transformed into yeast and 1728 clones (3.6 times the diversity) were screened for cleavage against the HprCH3.4 DNA target (ccatctcttgt_P; SEQ ID NO: 22). 10 positive clones were found, and after sequencing and validation by secondary screening 9 combinatorial mutants were identified (Table IV). The mutants are identified by an 11 letter code, corresponding to the amino acid residues at positions 28, 30, 32, 33, 38, 40, 44, 68, 70, 75 and 77. For example, KNSTYS/KYSEV stands for I-CreI K28, N30, S32, T33, Y38, S40, K44, Y68, S70, E75, and V77 (SEQ ID NO: 56).

Among these nine mutants, four corresponded to the bona fide assembly of 2 parental molecules (Table IV; SEQ ID NO: 52 to 55), whereas five others displayed non parental combinations at positions 28, 30, 32, 33, 38, 40 or 44, 68, 70, 75, 77. These five mutants are:

| | |
|---|---|
| KNSTYS/KYSEV | (SEQ ID NO: 56) |
| KNRDQS/KYSDR | (SEQ ID NO: 57) |
| KNSSDS/KYSDR | (SEQ ID NO: 58) |
| KNTHQS/KYSNR | (SEQ ID NO: 59) |
| KNSYQS/RYSNI | (SEQ ID NO: 60) |

Such mutants likely result from recombination between similar PCR fragments during the transformation process. Examples of positives are shown in FIG. 12.

TABLE IV
Cleavage of the HprCH3.4 target by mutants theoretically present in
the combinatorial library

| | | Amino acids at positions 28, 30, 32, 33, 38 and 40 | | | | | | | |
|---|---|---|---|---|---|---|---|---|---|
| | | (ex: KNRDQS stands for K28, N30, R32, D33, Q38 and S40) | | | | | | | |
| | | KNRDQS | KNTGQS | KNSQYS | KNSSDS | KSSQQS | KCSTQS | KNTHQS | KTSYQS |
| Amino acids at | GQTNI | | | | | | | | |
| positions 44, 68, | KASDK | | | | | | | | |
| 70, 75 and 77 | KASDV | | | | | | | | |
| (ex: GQTNI stands | KASNI | | | | | | | | |
| for G44, Q68, T70, | KESDK | | | | | | | | |
| N75 and I77) | KESDR | | | | | | | | |
| | KGSNI | | | | | | | | |
| | KNQNI | | | | | | | | |
| | KNSNI | | | | | | | | |
| | KQSNR | | | | | | | | |
| | KRDNI | | | | | | | | |
| | KRENI | | | | | | | | |
| | KRSDA | | | | | | | | |
| | KRSNV | | | | | | | | |
| | KRTNI | | | | | | | | |
| | KSSNI | | | | | | | | |
| | KSSNV | | | | | | | | |
| | KTQNI | | | | | | | | |
| | KTSDR | | | | | | | | |
| | KTSDV | | | | | | | | |
| | KTSNI | | | | | | | | |
| | KYSDI | | | | | | | | |
| | KYSDT | | | | | | | | |
| | KYSEV | | | | | | | | |
| | KYSNI | | | | | | | | |
| | KYSYN | | | | | | | | |
| | NHNNI | | | | | | | | |
| | NQRNI | | | | | | | | |
| | QASQR | | | | | | | | |
| | QASYR | | | | | | | | |
| | QESNR | | | | | | | | |
| | QNSQR | | | | | + | | | |
| | QRSHY | | | | | | | + | |
| | QRSNI | | | | | | | | |
| | QRSNK | | | | | | | | |
| | QRSQR | | | | | | | | |
| | QRSYR | | | | | | | | |
| | RASER | | | | | | | | |
| | RASNI | | | | | | | | |
| | RASNN | | | | | | | +++ | |
| | RESDR | | | | | | | | |
| | RNSDR | | | | | | | | |
| | RNSNN | | | | | | | | |
| | RQSNN | | | | | | | | |
| | RRSDQ | | | | | | | | |
| | RRSNN | | | | | | | | |
| | RSSER | | | | | | | | |
| | RTSER | | | | | | | | |
| | RTSNN | | | | | | | | |
| | RYGYI | | | | | | | | |
| | RYSDQ | | | | | | | | |
| | RYSDN | | | | | +++ | | +++ | |
| | RYSDR | | | | | | | +++ | |
| | RYSEI | | | | | | | | |
| | RYSER | | | | | | | | |
| | RYSHI | | | | | | | | |
| | RYSNI | | | | | | | | |
| | RYSNN | | | | | | | | |
| | RYSNQ | | | | | | | | |
| | RYSQY | | | | | | | | |

+ indicates a functional combination.

### EXAMPLE 6

### Making of Meganucleases Cleaving HprCH3.2 and HprCH3

I-CreI mutants able to cleave each of the palindromic HprCH3 derived targets (HprCH3.3 and HprCH3.4) were identified in example 4 and example 5. Pairs of such mutants (one cutting HprCH3.3 and one cutting HprCH3.4) were co-expressed in yeast. Upon co-expression, there should be three active molecular species, two homodimers, and one heterodimer. It was assayed whether the heterodimers that should be formed, cut the HprCH3 and HprCH3.2 targets.

### A) Materials and Methods

### a) Mutant Co-expression

The experimental procedures are as described in International PCT Application WO 2006/097854 and Arnould et al. J. Mol. Biol., 2006, 355, 443-458.

Briefly, yeast DNA was extracted from mutants cleaving the HprCH3.4 target using standard protocols and was used to transform E. coli. The resulting plasmid DNA was then used to transform yeast strains expressing a mutant cutting the HprCH3.3 target. Transformants were selected on −L Glu+G418 medium.

### b) Mating of Meganuclease Co-expressing Clones and Screening in Yeast

The experimental procedure is as described in example 4, except that a low gridding (about 4 spots/cm²) was used.

### B) Results:

Co-expression of mutants cleaving the HprCH3.3 and HprCH3.4 sequences resulted in efficient cleavage of the HprCH3.2 target in most cases (FIG. 13A). In addition, some of these combinations were able to cut the HprCH3 natural target that differs from the HprCH3.2 sequence by 4 by at positions −1, −2, 1 and 2. (FIG. 13B). Functional combinations are summarized in Table V and Table VI.

TABLE V
Cleavage of the HprCH3.2 target by the heterodimeric mutants

| | | Amino acids at positions 28, 30, 32, 33, 38, 40/44, 68, 70, 75 and 77 | | | | | | | | |
|---|---|---|---|---|---|---|---|---|---|---|
| | | of the I-CreI mutants cleaving the HprCH3.3 target | | | | | | | | |
| | | (ex: KNSCKS/QRRDI stands for K28, N30, S32, C33, K38, S40/Q44, R68, R70, D75 and I77) | | | | | | | | |
| | | KNSCKS/ | KNDTQS/ | KNSTSS/ | KNSTTS/ | KNTPQS/ | KNSCSS/ | KNTTQS/ | KNSHQS/ | KNSQQS/ |
| | | QRRDI | QRRDI | QRRDI | QRRDI | QRRDI | QRRDI | QRRDI | QRRDI | QRRDI |
| Amino acids at positions | KNTHQS/ | + | + | + | + | + | + | + | + | + |
| 28, 30, 32, 33, 38, 40/44, | RYSDN | | | | | | | | | |
| 68, 70, 75 and 77 Of I-CreI | KNSYQS/* | | + | + | + | + | + | + | + | + |
| mutants cleaving the | RYSNI | | | | | | | | | |
| HprCH3.4 target | KNSTYS/ | + | + | + | + | + | + | + | + | + |
| (ex: KNTHQS/RYSDN | KYSEV | | | | | | | | | |
| stands for K28, N30, T32, | KNTHQS/ | | + | | + | + | | + | + | + |
| H33, Q38, S40/R44, Y68, | KYSNR | | | | | | | | | |
| S70, D75 and N77) | KNRDQS/ | + | + | + | + | + | + | + | + | + |
| | KYSDR | | | | | | | | | |
| | KNSSDS/ | + | + | + | + | + | + | + | + | + |
| | KYSDR | | | | | | | | | |
| | KNTHQS/ | + | + | + | + | + | + | + | + | + |
| | RASNN | | | | | | | | | |
| | KNTHQS/ | | + | + | + | + | + | + | + | + |
| | RYSDR | | | | | | | | | |
| | KSSQQS/ | | + | + | + | + | + | + | + | + |
| | RYSDN | | | | | | | | | |

+ indicates a functional combination. Mutants in bold are mutants with alternative mutations in example 5.

TABLE VI
Cleavage of the HprCH3 target by the heterodimeric mutants

| | | Amino acids at positions 28, 30, 32, 33, 38, 40/44, 68, 70, 75 and 77 | | | | | | | | |
|---|---|---|---|---|---|---|---|---|---|---|
| | | of I-CreI mutants cleaving the HprCH3.3 target | | | | | | | | |
| | | (ex: KNSCKS/QRRDI stands for K28, N30, S32, C33, K38, S40/Q44, R68, R70, D75 and I77) | | | | | | | | |
| | | KNSCKS/ | KNDTQS/ | KNSTSS/ | KNSTTS/ | KNTPQS/ | KNSCSS/ | KNTTQS/ | KNSHQS/ | KNSQQS/ |
| | | QRRDI | QRRDI | QRRDI | QRRDI | QRRDI | QRRDI | QRRDI | QRRDI | QRRDI |
| Amino acids at positions | KNTHQS/ | | | | | | | | + | |
| 28, 30, 32, 33, 38, 40/44, | RYSDN | | | | | | | | | |
| 68, 70, 75 and 77 of I-CreI | KNSYQS/ | | | | | | | | + | |
| mutants cleaving the | RYSNI | | | | | | | | | |
| HprCH3.4 target | NSTYS/ | | | | | | | | + | |
| (ex: KNTHQS/RYSDN | KYSEV | | | | | | | | | |
| stands for K28, N30, T32, | KNTHQS/ | | | | | | | | | |
| H33, Q38, S40/R44, Y68, | KYSNR | | | | | | | | | |
| S70, D75 and N77) | KNRDQS/ | | | | | | | | + | |
| | KYSDR | | | | | | | | | |
| | KNSSDS/ | | | | | | | | + | |
| | KYSDR | | | | | | | | | |
| | KNTHQS/ | | | | | | | | + | |
| | RASNN | | | | | | | | | |
| | KNTHQS/ | | | | | | | | + | |
| | RYSDR | | | | | | | | | |
| | KSSQQS/ | | | | | | | | + | |
| | RYSDN | | | | | | | | | |

+ indicates a functional combination
*Mutants in bold are mutants with alternative mutations in example 5.

### EXAMPLE 7

### Improvement of Meganucleases Cleaving HprCH3 by Random Mutagenesis of Proteins Cleaving HprCH3.4 and Assembly with Proteins Cleaving HprCH3.3

I-CreI mutants able to cleave the HprCH3.2 and HprCH3 target by assembly of mutants cleaving the palindromic HprCH3.3 and HprCH3.4 target have been previously identified in example 4. However, these mutants display stronger activity with the HprCH3.2 target compared to the HprCH3 target.

Therefore the combinatorial mutants cleaving HprCH3 were mutagenized, and variants displaying stronger cleavage of this target were screened. According to the structure of the I-CreI protein bound to its target, there is no contact between the 4 central base pairs (positions −2 to 2) and the I-CreI protein (Chevalier et al., Nat. Struct. Biol., 2001, 8, 312-316; Chevalier and Stoddard, Nucleic Acids Res., 2001, 29, 3757-3774; Chevalier et al., J. Mol. Biol., 2003, 329, 253-269). Thus, it is difficult to rationally choose a set of positions to mutagenize, and mutagenesis was performed on the whole protein. Random mutagenesis results in high complexity libraries. Therefore, to limit the complexity of the variant libraries to be tested, only one of the two components of the heterodimers cleaving HprCH3 was mutagenized. Thus, in a first step, proteins cleaving HprCH3.4 were mutagenized, and in a second step, it was assessed whether they could cleave HprCH3 when co-expressed with a protein cleaving HprCH3.3.

### A) Material and Methods

### a) Construction of Libraries by Random Mutagenesis

Random mutagenesis was performed on a pool of chosen mutants, by PCR using Mn²⁺ or by a two-step PCR process using dNTP derivatives 8-oxo-dGTP and dPTP as described in the protocol from Jena Bioscience GmbH for the JBS dNTP-Mutagenis kit. Primers used were preATGCreFor (5′-gcataaattactatacttctatagacacgcaaacacaaatacacagcggccttgccacc-3′: SEQ ID NO: 61) and ICreIpostRev (5′-ggctcgaggagctcgtctagaggatcgctcgagttatcagtcggccgc-3′: SEQ ID NO: 62). Approximately 25 ng of the PCR product and 75 ng of vector DNA (pCLS1107, FIG. 18) linearized by digestion with DraIII and NgoMIV were used to transform the yeast Saccharomyces cerevisiae strain FYC2-6A (MATα, trp1Δ63, leu2Δ1, his3Δ200) using a high efficiency LiAc transformation protocol (Gietz and Woods, Methods Enzymol., 2002, 350, 87-96). Expression plasmids containing an intact coding sequence for the I-CreI mutant were generated by in vivo homologous recombination in yeast.

### b) Mutant-Target Yeast Strains, Screening and Sequencing

The yeast strain FYBL2-7B (MAT a, ura3Δ851, trp1Δ63, leu2Δ1, lys2Δ202) containing the HprCH3 target in the yeast reporter vector (pCLS1055, FIG. 16) was transformed with mutants, in the leucine vector (pCLS0542), cutting the HprCH3.3 target, using a high efficiency LiAc transformation protocol. Mutant-target yeasts were used as target strains for mating assays as described in example 4. Positives resulting clones were verified by sequencing (MILLEGEN) as described in example 4.

### B) Results:

Four mutants cleaving HprCH3.4 (I-CreI 32T,33H,44K,68Y,70S,75N,77R, I-CreI 30S,33Q,44R,68Y,70S,77N, I-CreI 32T,33H,70S,75H,77Y and I-CreI 32T,33H,68N,70S,75Q,77R, also called KNTHQS/KYSNR, KSSQQS/RYSDN, KNTHQS/QRSHY and KNTHQS/QNSQR according to the nomenclature of Table IV; SEQ ID NO: 59, 53, 63 and 64) were pooled, randomly mutagenized and transformed into yeast. 1140 transformed clones were then mated with a yeast strain that contains (i) the HprCH3 target in a reporter plasmid (ii) an expression plasmid containing a mutant that cleaves the HprCH3.3 target (I-CreI 3311 or KNSHQS/QRRDI; SEQ ID NO: 32). After mating with this yeast strain, 23 clones were found to cleave the HprCH3 target more efficiently than the original mutant. Thus, 23 positives contained proteins able to form heterodimers with KNSHQS/QRRDI with strong cleavage activity for the HprCH3 target. An example of positives is shown in FIG. 14. Sequencing of these 23 positive clones indicates that 10 distinct mutants listed in Table VII were identified.

TABLE VII
Functional mutant combinations
displaying strong cleavage activity for HprCH3.

| | | Optimized Mutants HprCH3.4 (SEQ ID NO: 65 to 74) |
|---|---|---|
| Mutant | I-CreI 28K30N32S33H38Q40S44Q68R70R75D77I | I-CreI 28R, 30N, 32S, 33S, 38Y, 40Q, 44R, 68A, 70S, 75N, 77N |
| HprCH3.3 | (KNSHQS/QRRDI) | I-CreI 28R, 30N, 32S, 33S, 38Y, 40Q, 44R, 68A, 70S, 75H, 77Y |
| | | I-CreI 28R, 30N, 32T, 33S, 38Y, 40Q, 44R, 68Y, 70S, 75N, 77N, 140M |
| | | I-CreI 28K, 30N, 32T, 33H, 38H, 40S, 44Q, 68Y, 70S, 75D, 77R |
| | | I-CreI 28K, 30N, 32T, 33H, 38Q, 40S, 44K, 68Y, 70S, 75D, 77R |
| | | I-CreI 28K, 30N, 32T, 33H, 38Q, 40S, 44Q, 68N, 70S, 75H, 77R |
| | | I-CreI 28K, 30N, 32T, 33H, 38Q, 40S, 44Q, 68R, 70S, 75H, 77R |
| | | I-CreI 28K, 30N, 32T, 33H, 38Q, 40S, 44Q, 68H, 70S, 75H, 77H |
| | | I-CreI 28K, 30N, 32T, 33H, 38Q, 40S, 44Q, 68H, 70S, 75H, 77H, 92R |
| | | I-CreI 28K, 30N, 32T, 33H, 38Q, 40S, 44K, 68Y, 70S, 75D, 77R, 92R, |
| | | 96R, 107R, 132V, 140A, 143A |

### EXAMPLE 8

### Improvement of Meganucleases Cleaving HprCH3 by Random Mutagenesis of Proteins Cleaving HprCH3.3 and Assembly with Proteins Cleaving HprCH3.4

As a complement to example 6, it was also decided to perform random mutagenesis with the mutants that cleave HprCH3.3. Only one HprCh3.3 mutant was capable of cleaving HprCH3 so this mutant and three other mutants that strongly cleave HprCH3.3 but not HprCH3 were used for random mutagenesis.

The mutagenized proteins cleaving HprCH3.3 were then tested to determine if they could efficiently cleave HprCH3 when co-expressed with a protein cleaving HprCH3.4.

### A) Material and Methods

### a) Construction of Libraries by Random Mutagenesis

Random mutagenesis was performed on a pool of chosen mutants, by PCR using Mn²⁺ or by a two-step PCR process using dNTP derivatives 8-oxo-dGTP and dPTP as described in the protocol from Jena Bioscience GmbH for the JBS dNTP-Mutageneis kit. Primers used were preATGCreFor (5′-gcataaattactatacttctatagacacgcaaacacaaatacacagcggccttgccacc-3′: SEQ ID NO: 61) and ICreIpostRev (5′-ggctcgaggagctcgtctagaggatcgctcgagttatcagtcggccgc-3′: SEQ ID NO: 62). Approximately 25 ng of the PCR product and 75 ng of vector DNA (pCLS0542, FIG. 17) linearized by digestion with NcoI and EagI were used to transform the yeast Saccharomyces cerevisiae strain FYC2-6A (MATα, trp1Δ63, leu2Δ1, his3Δ200) using a high efficiency LiAc transformation protocol (Gietz and Woods, Methods Enzymol., 2002, 350, 87-96). Expression plasmids containing an intact coding sequence for the I-CreI mutant were generated by in vivo homologous recombination in yeast.

### b) Mutant-Target Yeast Strains, Screening and Sequencing

The yeast strain FYBL2-7B (MAT a, ura3Δ851, trp1Δ63, leu2Δ1, lys2Δ202) containing the HprCH3 target in the yeast reporter vector (pCLS1055, FIG. 16) was transformed with mutants, in the kanamycin resistant vector (pCLS 1107), cutting the HprCH3.4 target, using a high efficiency LiAc transformation protocol. Mutant-target yeasts were used as target strains for mating assays as described in example 6. Positives resulting clones were verified by sequencing (MILLEGEN) as described in example 4.

### B) Results

Four mutants cleaving HprCH3.3 (I-CreI 32D,33T, I-CreI 32T,33T, I-CreI 33H and I-CreI 33Q, also called KNDTQS/QRRDI, KNTTQS/QRRDI, KNSHQS/QRRDI and KNSQQS/QRRDI according to the nomenclature of Table IV; SEQ ID NO: 24, 45, 32 and 35) were pooled, randomly mutagenized and transformed into yeast. 1140 transformed clones were then mated with a yeast strain that contains (i) the HprCH3 target in a reporter plasmid (ii) an expression plasmid containing a mutant that cleaves the HprCH3.4 target (I-CreI 33T,38Y,44K,68Y,70S,75E,77V or KNSTYS/KYSEV; SEQ ID NO: 56). After mating with this yeast strain, 18 clones were found to efficiently cleave the HprCH3 target. Thus, 18 positives contained proteins able to form heterodimers with KNSTYS/KYSEV with cleavage activity for the HprCH3 target. An example of positives is shown in FIG. 15. Examples of such heterodimeric mutants are listed in Table VIII.

TABLE VIII
Functional mutant combinations displaying cleavage activity for
HprCH3

| | | Optimized Mutants HprCH3.3 (SEQ ID NO: 75 to 82) |
|---|---|---|
| Mutant HprCH3.4 | I-CreI 28K30N32S33T38Y40S44M68Y70S75E77V | I-CreI 33H 66C 137V 155R 162P |
| | (KNSTYS/KYSEV) | I-CreI 9L 33H 100I 108V 154G 155P 161P |
| | | I-CreI 2Y 33H 109V 125A |
| | | I-CreI 33H 113S 136S |
| | | I-CreI 2I 33H 81V 86I 110G 131R 135Q 151A 157V |
| | | I-CreI 33H 71R 103I 129A 130G |
| | | I-CreI 33H 69V 82R 90R 120V 139R 158M |
| | | I-CreI 33H 54L 86D 100R 104M 105A 136S 159R |

### EXAMPLE 9

### Refinement of Meganucleases Cleaving the HprCH3 Target Site by Site-Directed Mutagenesis Resulting in the Substitution of Glycine-19 with Serine (G19S)

To validate the ability of the G19S substitution to increase the cleavage activity of I-CreI derived meganucleases, this mutation was incorporated into each of the two proteins of the heterodimer HprCH3.3 (KNSHQS/QRRDI/42A43L, SEQ ID NO: 147)/HprCH3.4 (KNTHQS/RYSNN/72T, SEQ ID NO: 148). This heterodimer which cleaves the HprCH3 target was obtained by random mutagenesis, as described in examples 7 and 8.

To evaluate the cleavage activity of the original and G19S derived mutants a chromosomal reporter system in CHO cells was used (FIG. 20). In this system a single-copy LacZ gene driven by the CMV promoter is interrupted by the HprCH3 site and is thus non-functional. The transfection of the cell line with CHO expression plasmids for HprCH3.3/HprCH3.4 and a LacZ repair plasmid allows the restoration of a functional LacZ gene by homologous recombination. It has previously been shown that double-strand breaks can induce homologous recombination; therefore the frequency with which the LacZ gene is repaired is indicative of the cleavage efficiency of the genomic HprCH3 target site.

### 1) Material and Methods

### a) Site-Directed Mutagenesis

To introduce the G19S substitution into the HprCH3.3 and HprCH3.4 coding sequences, two separate overlapping PCR reactions were carried out that amplify the 5′ end (residues 1-24) or the 3′ end (residues 14-167) of the I-CreI coding sequence. For both the 5′ and 3′ end, PCR amplification is carried out using a primer with homology to the vector: CCM2For 5′-aagcagagactctggctaactagagaacccactgcttactggcttatcgaccatggccaatacca aatataacaaagagttcc-3’ (SEQ ID NO: 149) or CCMRev 5′-tctgatcgattcaagtcagtgtctctctag atagcgagtcggccgccggggaggatttcttcttctcgc -3′: SEQ ID NO: 150) and a primer specific to the I-CreI coding sequence for amino acids 14-24 that contains the substitution mutation G19S: G19SF 5′-gccggctttgtggactctgacggtagcatcatc-3′ (SEQ ID NO:151) or G19SR 5′-gatgatgctaccgtcagagtccacaaagccggc-3′ (SEQ ID NO: 152).

The resulting PCR products contain 33 by of homology with each other. Subsequently the fragments are assembled by PCR using an aliquot of each of the two fragments and the CCM2For and CCMRev primers.

### b) Cloning of Mutants in a CHO Expression Vector

PCR products digested with SacI-XbaI were cloned into the corresponding SacI-XbaI sites of the plasmid pCLS1088 (FIG. 21), a CHO gateway expression vector pCDNA6.2 (INVITROGEN) containing the I-CreI N75 coding sequence. The substitution of the HprCH3.3-G19S or HprCH3.4-G19S coding sequence for the I-CreI N75 coding sequence was verified by sequencing (MILLEGEN).

### c) Chromosomal Assay in Mammalian Cells

CHO cell lines harbouring the reporter system were seeded at a density of 2×10⁵ cells per 10 cm dish in complete medium (Kaighn's modified F-12 medium (F12-K), supplemented with 2 mM L-glutamine, penicillin (100 UI/ml), streptomycin (100 μg/ml), amphotericin B (Fongizone) (0.25 μg/ml) (INVITROGEN-LIFE SCIENCE) and 10% FBS (SIGMA-ALDRICH CHIMIE). The next day, cells were transfected with Polyfect transfection reagent (QIAGEN). Briefly, 2 μg of lacz repair matrix vector was co-transfected with various amounts of meganucleases expression vectors. After 72 hours of incubation at 37° C., cells were fixed in 0.5% glutaraldehyde at 4° C. for 10 min, washed twice in 100 mM phosphate buffer with 0.02% NP40 and stained with the following staining buffer (10 mM Phosphate buffer, 1 mM MgCl₂, 33 mM K hexacyanoferrate (III), 33 mM K hexacyanoferrate (II), 0.1% (v/v) X-Gal). After, an overnight incubation at 37° C., plates were examined under a light microscope and the number of LacZ positive cell clones counted. The frequency of LacZ repair is expressed as the number of LacZ+ foci divided by the number of transfected cells (5×10⁵) and corrected by the transfection efficiency.

### 2) Results

The activities of heterodimers containing either the two initial mutants (HprCH3.3/HprCH3.4) or one of the two G19S derived mutants combined with the corresponding initial mutant (HprCH3.3/HprCh3.4 G19S or HprCH3.3 G19S/HprCh3.4) were tested using a chromosomal assay in a CHO cell line containing the HprCH3 target. This chromosomal assay has been extensively described in a recent publication (Arnould et al., J. Mol. Biol. Epub May 10, 2007). Briefly, a CHO cell line carrying a single copy transgene was first created. The transgene contains a human EF1α promoter upstream an I-SceI cleavage site (FIG. 20, step 1). Second, the I-SceI meganuclease was used to trigger DSB-induced homologous recombination at this locus, and insert a 5.5 kb cassette with a novel meganuclease cleavage site (FIG. 20, step2). This cassette contains a non functional LacZ open reading frame driven by a CMV promoter, and a promoter-less hygromycin marker gene. The LacZ gene itself is inactivated by a 50 by insertion containing the meganuclease cleavage site to be tested (here, the HprCH3 cleavage site). This cell lines can in turn be used to evaluate DSB-induced gene targeting efficiencies (LacZ repair) with engineered I-CreI derivatives cleaving the HprCH3 target (FIG. 20, step3).

This cell line was co-transfected with the repair matrix and various amounts of the vectors expressing the meganucleases. The frequency of repair of the LacZ gene increased from a maximum of 2.0×10⁻³ with the initial engineered heterodimers (HprCH3.3/HprCH3.4), to a maximum of 1.15×10⁻² with the HprCH3.3-G19S derived mutant and a maximum of 1.2×10⁻² with the HprCH3.4-G19S derived mutant (FIG. 22).

These finding demonstrates that G19S mutation is able, by itself, to enhance the activity of an heterodimer, when found in only one of its monomers. A single G19S substitution was shown to enhance the activity of completely different heterodimers, cleaving other targets. Thus, the G19S mutation behaves like a “portable” motif, able to enhance the activity of different I-CreI derivatives by itself, or in combinations with other mutations.

However, when the HprCH3.3 G19S/HprCH3.4 G19S heterodimer was transformed with the repair matrix, no LacZ+ foci were detected, indicating a recombination frequency of less than 6.0×10⁻⁶. These finding indicate that a single G19S substitution enhances the activity, a G19S substitution in each monomers of the heterodimer results in a very strong decrease of the activity.

1. A method for inducing a site-specific modification in the HPRT gene, for a non-therapeutic purpose, by adding an I-CreI variant or single-chain derivative having at least one substitution in one of the two functional subdomains of the LAGLIDADG (SEQ ID NO: 153) core domain situated from positions 26 to 40 and 44 to 77 of I-CreI to a DNA target sequence selected from the group consisting of the sequences SEQ ID NO: 1 to 14 thereby cleaving the DNA target.

2. The method according to claim 1, wherein said HPRT gene is a non-human mammal HPRT gene.

3. The method according to claim 2, wherein said HPRT gene is the Criteculus sp. HPRT gene.

4. The method according to claim 2, wherein said HPRT gene is the the Mus musculus HPRT gene.

5. The method according to claim 4, wherein said I-CreI variant cleaves a DNA target of the sequence SEQ ID NO: 6, 7, 8, 9, 10, 11, 12 or 14.

6. The method according to claim 1, wherein said HPRT gene is the Homo sapiens HPRT gene.

7. The method according to claim 6, wherein said I-CreI variant cleaves a DNA target of the sequence SEQ ID NO: 6, 8, 9, 12 or 14.

8. The method according to claim 1, wherein said I-CreI variant or single-chain derivative is combined with a targeting DNA construct comprising a sequence to be introduced flanked by sequences sharing homologies with the regions of the HPRT gene surrounding the genomic DNA cleavage site of said I-CreI variant or single chain derivative.

9. The method according to claim 8, wherein said sequence to be introduced comprises a gene of interest.

10. The method according to claim 8, wherein said sequence to be introduced comprises an inactivation cassette for the HPRT gene.

11. The method according to claim 8, wherein said targeting DNA construct is inserted in a vector.

12. The method according to claim 1, wherein said I-CreI variant or single-chain derivative is encoded by a polynucleotide fragment.

13. The method according to claim 12, wherein said fragment is inserted in an expression vector.

14. The method according to claim 13, wherein said vector comprises two different polynucleotide fragments, each encoding one of the monomers of an heterodimeric I-Cre I variant.

15. The method according to claim 13, wherein said vector includes a targeting DNA construct comprising a sequence to be introduced flanked by sequences sharing homologies with the regions of the HPRT gene surrounding the genomic DNA cleavage site of said I-Cre I variant or single chain derivative.

16. The method according to claim 1, wherein said site-specific modification of the HPRT gene consists in the insertion of a gene of interest by cleavage of the HPRT gene by said I-CreI variant and homologous recombination with a targeting DNA construct comprising a gene of interest.

17. The method according to claim 1, wherein said site-specific modification of the HPRT gene consists in the insertion of an inactivation cassette by cleavage of the HPRT gene by said I-CreI variant and homologous recombination with a targeting DNA construct comprising an inactivation cassette for the HPRT gene.

18. The method according to claim 1, wherein said site-specific modification of the HPRT gene consists in the inactivation of the HPRT gene by cleavage of the HPRT gene by said I-CreI variant and repair of the double-strands break by non-homologous end joining.

19. The method according to claim 1, for making non-human transgenic animals or recombinant cell lines.

20. The method according to claim 19, for making recombinant human cell lines.

21. The method according to claim 1, wherein said I-CreI variant is obtained by a method comprising:
(a) constructing a first series of I-CreI variants having at least one substitution in a first functional subdomain of the LAGLIDADG (SEQ ID NO: 153) core domain situated from positions 26 to 40 of I-CreI,
(b) constructing a second series of I-CreI variants having at least one substitution in a second functional subdomain of the LAGLIDADG (SEQ ID NO: 153) core domain situated from positions 44 to 77 of I-CreI,
(c) selecting and/or screening the variants from the first series of (a) which are able to cleave a mutant I-CreI site wherein (i) the nucleotide triplet in positions −10 to −8 of the I-CreI site has been replaced with a nucleotide triplet selected from the group consisting of cag, att, cct, ttg, gac, atg, ttt, ttc, tgg, gtc, aag, gag and (ii) the nucleotide triplet in positions +8 to +10 has been replaced with the reverse complementary sequence of said nucleotide triplet which is substituted in position −10 to −8 of said I-CreI site and is selected from the group consisting of ctg, aat, agg, caa, gtc, cat, aaa, gaa, cca, gac, ctt, and ctc, respectively,
(d) selecting and/or screening the variants from the second series of (b) which are able to cleave a mutant I-CreI site wherein (i) the nucleotide triplet in positions −5 to −3 of the I-CreI site has been replaced with a nucleotide triplet selected from the group consisting of gac, taa, tca, gtg, gct, tgt, tgg, ctg, ttg, tag, and gag and (ii) the nucleotide triplet in positions +3 to +5 has been replaced with the reverse complementary sequence of said nucleotide triplet which is substituted in position −5 to −3 of said I-CreI site and is selected from the group consisting of gtc, tta, tga, cac, agc, aca, cca, cag, caa, cta and ctc, respectively,
(e) selecting and/or screening the variants from the first series of (a) which are able to cleave a mutant I-CreI site wherein (i) the nucleotide triplet in positions +8 to +10 of the I-CreI site has been replaced with a nucleotide triplet selected from the group consisting of cat, cga, tat, ggg, tac, taa, cag, gca, aca, gaa, tga, atg, and (ii) the nucleotide triplet in positions −10 to −8 has been replaced with the reverse complementary sequence of said nucleotide triplet which is substituted in position +8 to +10 of said I-CreI site and is selected from the group consisting of atg, tcg, ata, ccc, gta, tta, ctg, tgc, tgt, ttc, tca and cat, respectively,
(f) selecting and/or screening the variants from the second series of (b) which are able to cleave a mutant I-CreI site wherein (i) the nucleotide triplet in positions +3 to +5 of the I-CreI site has been replaced with the nucleotide triplet selected from the group consisting of tcc, tat, gtg, gaa, tgg, tac, ttt, aca, agc, gcg, tcc, act, caa and aag and (ii) the nucleotide triplet in positions −5 to −3 has been replaced with the reverse complementary sequence of which is substituted in position +3 to +5 of said I-CreI site and is selected from the group consisting of gga, ata, cac, ttc, cca, gta, aaa, tgt, gct, cgc, gga, agt, ttg and ctt, respectively, and
(gᵢ) selecting and/or screening the variants from (c) to (f) which are able to cleave a DNA target of the sequence SEQ ID NO: 1 to 14.

22. The method according to claim 21, wherein said I-CreI variant is obtained by a method comprising at least (a) to (f), and
(g₂) combining different variants obtained in any of (c) to (f) with each other or with I-CreI, to form heterodimers, and
(h₂) selecting and/or screening the heterodimers from (g₂) which are able to cleave said DNA target of the sequence SEQ ID NO: 1 to 14.

23. The method according to claim 21, wherein said I-CreI variant is obtained by a method comprising at least (a) to (f), and
(g₃) combining in a single variant, the mutation(s) in positions 26 to 40 and 44 to 77 of two variants from (c) and (d), to obtain a novel homodimeric I-CreI variant which cleaves a sequence wherein (i) the nucleotide triplet in positions −10 to −8 is identical to the nucleotide triplet which is present in positions −10 to −8 of said DNA target of the sequence SEQ ID NO: 1 to 14, (ii) the nucleotide triplet in positions +8 to +10 is identical to the reverse complementary sequence of the nucleotide triplet which is present in positions −10 to −8 of said DNA target of the sequence SEQ ID NO: 1 to 14, (iii) the nucleotide triplet in positions −5 to −3 is identical to the nucleotide triplet which is present in positions −5 to −3 of said DNA target of the sequence SEQ ID NO: 1 to 14 and (iv) the nucleotide triplet in positions +3 to +5 is identical to the reverse complementary sequence of the nucleotide triplet which is present in positions −5 to −3 of said of said DNA target of the sequence SEQ ID NO: 1 to 14, and/or,
(h₃) combining in a single variant, the mutation(s) in positions 26 to 40 and 44 to 77 of two variants from (e) and (0, to obtain a novel homodimeric I-CreI variant which cleaves a sequence wherein (i) the nucleotide triplet in positions +3 to +5 is identical to the nucleotide triplet which is present in positions +3 to +5 of said of said DNA target of the sequence SEQ ID NO: 1 to 14, (ii) the nucleotide triplet in positions −5 to −3 is identical to the reverse complementary sequence of the nucleotide triplet which is present in positions +3 to +5 of said of said DNA target of the sequence SEQ ID NO: 1 to 14, (iii) the nucleotide triplet in positions +8 to +10 of the I-CreI site has been replaced with the nucleotide triplet which is present in positions +8 to +10 of said of said DNA target of the sequence SEQ ID NO: 1 to 14 and (iv) the nucleotide triplet in positions −10 to −8 is identical to the reverse complementary sequence of the nucleotide triplet in positions +8 to +10 of said of said DNA target of the sequence SEQ ID NO: 1 to 14, and
(i₃) selecting and/or screening the variants from (g₃) or (h₃) which are able to cleave a DNA target of the sequence SEQ ID NO: 1 to 14.

24. The method, wherein said I-CreI variant is obtained by a method comprising (a) to (f) (g₃) and/or (h₃) as defined in claim 23, and
(i₄) combining the variants obtained in (g₃) with the variants obtained in (h₃), I-CreI or the variants obtained in (e) or (f), to form heterodimers, or
(i′₄) combining the variants obtained in (h₃) with I-CreI or the variants obtained in (c) or (d), to form heterodimers, and
(j₄) selecting and/or screening the heterodimers from (i₄) or (i′₄) which are able to cleave a DNA target of the sequence SEQ ID NO: 1 to 14.

25. The method according to claim 1, wherein said substitution(s) in the subdomain situated from positions 44 to 77 of I-CreI are in positions 44, 68, 70, 75 and/or 77.

26. The method according to claim 1, wherein said substitution(s) in the subdomain situated from positions 26 to 40 of I-CreI are in positions 26, 28, 30, 32, 33, 38 and/or 40.

27. The method according to claim 1, wherein said I-CreI variant comprises one or more additional substitution(s) in I-CreI.

28. The method according to claim 27, wherein said substitutions are at positions: 2, 9, 19, 42, 43, 54, 66, 69, 72, 81, 82, 86, 90, 92, 96, 100, 103, 104, 105, 107, 108, 109, 110, 113, 120, 125, 129, 130, 131, 132, 135, 136, 137, 140, 143, 151, 154, 155, 157, 158, 159, 161 or 162 of I-CreI.

29. The method according to claim 1, wherein said substitutions are replacement of the initial amino acids with amino acids selected from the group consisting of A, C, D, E, G, H, K, N, P, Q, R, S, T, Y, W, L, V, M and I.

30. The method according to claim 28, wherein said substitution is the G19S or G19A mutation.

31. The method according to claim 1, wherein said I-CreI variant is an heterodimer, resulting from the association of a first and a second monomer having different mutations in positions 26 to 40 and/or 44 to 77 of I-CreI.

32. The method according to claim 31, wherein at least one monomer has at least two substitutions, one in each of the two functional subdomains situated from positions 26 to 40 and 44 to 77 of I-CreI.

33. The method according to claim 32, wherein said heterodimer consist of a first and a second monomer selected from the following pairs of sequences: SEQ ID NO: 83 and 97, SEQ ID NO: 84 and 98, SEQ ID NO: 85 and 99, SEQ ID NO: 32 and 52, SEQ ID NO: 32 and 53, SEQ ID NO: 32 and 54, SEQ ID NO: 32 and 55, SEQ ID NO: 32 and 56, SEQ ID NO: 32 and 57, SEQ ID NO: 32 and 58, SEQ ID NO: 32 and 60, SEQ ID NO: 32 and 65, SEQ ID NO: 32 and 66, SEQ ID NO: 32 and 67, SEQ ID NO: 32 and 68, SEQ ID NO: 32 and 69, SEQ ID NO: 32 and 70, SEQ ID NO: 32 and 71, SEQ ID NO: 32 and 72, SEQ ID NO: 32 and 73, SEQ ID NO: 32 and 74, SEQ ID NO: 75 and 56, SEQ ID NO: 76 and 56, SEQ ID NO: 77 and 56, SEQ ID NO: 78 and 56, SEQ ID NO: 79 and 56, SEQ ID NO: 80 and 56, SEQ ID NO: 81 and 56, SEQ ID NO: 82 and 56, SEQ ID NO: 86 and 96, SEQ ID NO: 87 and 100, SEQ ID NO: 88 and 101, SEQ ID NO: 89 and 102, SEQ ID NO: 90 and 103, SEQ ID NO: 91 and 104, SEQ ID NO: 92 and 105, SEQ ID NO: 93 and 106, SEQ ID NO: 94 and 107, SEQ ID NO: 95 and 108, and SEQ ID NO: 147 and 148.

34. The method according to claim 31, wherein one monomer of the heterodimer comprises the G19S mutation.

35. A medicament for preventing, improving or curing a genetic disease associated with a mutation in the HPRT gene comprising an I-CreI variant or a single-chain derivative prepared by the method according to claim 21.

36. The medicament according to claim 35, wherein said I-CreI variant or single-chain derivative is associated with a targeting DNA construct comprising a sequence which repairs a mutation in the HPRT gene flanked by sequences sharing homologies with the regions of the HPRT gene surrounding the genomic DNA target of said I-CreI variant or single-chain derivative.

37. The medicament according to claim 36, wherein said sequence which repairs said mutation is the correct sequence of the HPRT gene.

38. The medicament according to claim 36, wherein said sequence which repairs said mutation comprises the exons of the HPRT downstream of the genomic cleavage site fused in frame, and a polyadenylation site to stop transcription in 3′.

39. The medicament according to claim 36, wherein the I-CreI variant or single-chain derivative is encoded by a vector comprising the targeting DNA construct.

40. The medicament according to claim 35, wherein said genetic disease is the Lesch Nyhan Syndrome.

41. An I-CreI variant as prepared according to claim 21.

42. A single-chain chimeric endonuclease derived from an I-CreI variant according to claim 41.

43. A polynucleotide fragment encoding a variant according to claim 41.

44. An expression vector comprising at least one polynucleotide fragment according to claim 43.

45. The expression vector according to claim 44, which comprises two different polynucleotide fragments, each encoding one of the monomers of an heterodimeric I-CreI variant.

46. The expression vector according to claim 44, which comprises a targeting DNA construct comprising a sequence to be introduced flanked by sequences sharing homologies with the regions of the HPRT gene surrounding the genomic DNA cleavage site of said I-Cre I variant or single chain derivative.

47. A vector comprising a targeting DNA construct comprising a sequence to be introduced flanked by sequences sharing homologies with the regions of the HPRT gene surrounding the genomic DNA cleavage site of said I-Cre I variant or single chain derivative.

48. A host cell which is modified by a polynucleotide according to claim 41.

49. A non-human transgenic animal comprising one or two polynucleotide fragments as defined in claim 43.

50. A transgenic plant comprising one or two polynucleotide fragments as defined in claim 43.

51. A composition comprising at least one I-CreI variant as defined in claim 42.
